(19) United States
(12) Patent Application Publication
(10) Pub. No.: US 2026/0191982 A1
(43) Pub. Date: Jul. 9, 2026

(54) ANTI-BASAL CELL ADHESION MOLECULE ANTIBODY-DRUG CONJUGATE

(71) Applicants: Genome & Company, Inc., Suwon-si, Gyeonggi-do (KR); Debiopharm Research & Manufacturing SA, Martigny (CH)

(72) Inventors: Mi Young Cha, Seongnam-Si, Gyeonggi-do (KR); Hyunkyung Yu, Seongnam-Si, Gyeonggi-do (KR); Hyunuk Kim, Seongnam-Si, Gyeonggi-do (KR); Youngeun Ha, Seongnam-Si, Gyeonggi-do (KR); Kitae Park, Seongnam-Si, Gyeonggi-do (KR); Bunam Jeon, Seongnam-Si, Gyeonggi-do (KR); Yun Yeon KIM, Seongnam-Si, Gyeonggi-do (KR); Léo Marx, Martigny (CH); Mathilde Pantin, Thonon-les-bains (FR); Nathalie Bellocq, Fully (CH); Frederic Levy, Lausanne (CH)

(73) Assignees: Genome & Company, Inc., Suwon-si, Gyeonggi-do (KR); Debiopharm Research & Manufacturing SA, Martigny (CH)

(21) Appl. No.: 19/130,894

(22) PCT Filed: Nov. 17, 2023
(86) PCT No.: PCT/KR2023/018574

§ 371 Date: May 16, 2025

(30) Foreign Application Priority Data
Nov. 18, 2022 (KR) 10-2022-0155122

(51) Int. Cl. A61K 47/68; A61P 35/00

(52) U.S. Cl. CPC A61K 47/6849; A61K 47/68031; A61K 47/68033; A61K 47/68037; A61K 47/6889; A61P 35/00

(57) ABSTRACT

The present disclosure relates to an anti-basal cell adhesion molecule (BCAM) antibody-drug conjugate where the anti-BCAM antibody is conjugated to an antitumor compound via a linker, a pharmaceutical composition comprising the anti-BCAM antibody-drug conjugate, and a method for treating cancer by administering an effective amount of the anti-BCAM antibody-drug conjugate to a subject in need thereof.

### TECHNICAL FIELD

### Cross Reference to Related Applications

This application claims priority to KR patent application No. 10-2022-0155122 filed Nov. 18, 2022, entitled “ANTI-BASAL CELL ADHESION MOLECULE ANTIBODY-DRUG CONJUGATE”, which is incorporated herein by reference in its entirety.

### FIELD

The present disclosure relates to an anti-basal cell adhesion molecule (BCAM) antibody-drug conjugate where the anti-BCAM antibody is conjugated to an antitumor compound via a linker, a pharmaceutical composition comprising the anti-BCAM antibody-drug conjugate, and a method for treating cancer by administering an effective amount of the anti-BCAM antibody-drug conjugate to a subject in need thereof.

### BACKGROUND ART

### Background

Basal cell adhesion molecule (BCAM) is a member of the immunoglobulin superfamily and a receptor for laminin which facilitates cell adhesion, migration, and invasion. It has been reported that BCAM plays an essential role in tumor progression and is overexpressed in certain cancers. It is also known as Lutheran antigen (LU). Antibody-drug conjugate (ADC) is composed of an antibody covalently attached to an antitumor drug via a linker, and combines the selectivity of antibody and the cytotoxic properties of the antitumor drug using the chemical linker. There has been a need for anti-BCAM antibody-drug conjugate that can be used for therapeutic purposes in the treatment of cancer.

### DISCLOSURE OF INVENTION

### Technical Problem

### Summary

The present disclosure provides an antibody-drug conjugate of Formula (1) having the structure of Ab-(L-(D)ₘ)ₙ or a pharmaceutically acceptable salt thereof. In Formula (1), Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR 2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6. In addition, D is an antitumor compound which is conjugated to the anti-BCAM antibody or antibody fragment via the linker, and L is a linker represented by Formula (2):

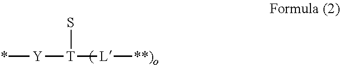

wherein Y is a divalent group comprising one or more atoms selected from C, N, O, P and S,
wherein T is a (1+o)- or (2+o)-valent connecting group,
wherein S is an atom or group that is optionally present to saturate a free valency of T,
wherein L′ is a linker capable of being cleaved by Cathepsin B,
wherein o is an integer of 1 to 5,
wherein * indicates covalent attachment to the anti-BCAM antibody (Ab), and
wherein ** indicates covalent attachment to one or more antitumor compounds (D).

In Formula (1), n is 1 to 10, and m is 1 to 5. In one embodiment, in the anti-BCAM antibody, the heavy chain variable region comprises a sequence of SEQ ID NO: 7 and the light chain variable region comprises a sequence of SEQ ID NO: 8. In some embodiment, n is from 3 to 8 and m is 1. In some embodiment, a drug (the antitumor compound) to antibody (the anti-BCAM antibody) ratio (DAR) is from about 3 to about 8. In another embodiment, the DAR is about 4. In addition, the antigen binding fragment thereof may be an antibody fragment selected from the group consisting of a Fab fragment, a Fab′ fragment, a Fab′-SH, a Fv fragment, a scFv fragment, a F(ab′)₂ fragment, a VL fragment, a VH fragment, a ScFv-Fc fragment, and a (ScFv)₂-Fc fragment, a diabody, a linear antibody, a fragment produced by a Fab expression library, an anti-idiotypic (anti-Id) antibody, a complementary determining region (CDR), and an epitope-binding fragment. In another embodiment, the anti-BCAM antibody is a chimeric antibody, a humanized antibody, or a human antibody.

In one embodiment, the antibody according to the present disclosure may be IgG1, IgG2, IgG3, IgG4 or a mutant thereof. In another embodiment, the antibody of the present disclosure may be IgG1, IgG4 or a mutant thereof. The present disclosure also provides a pharmaceutical composition comprising the antibody-drug conjugate of Formula (1) and a pharmaceutically acceptable carrier. In one embodiment, the pharmaceutical composition is for treating cancer. In an embodiment, the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

The present disclosure also provides a method for treating cancer, the method comprising administering an effective amount of the antibody-drug conjugate to a subject in need thereof. In one embodiment, the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, kidney cancer, head and neck cancer, hypopharyngeal squamous cell carcinoma, glioblastoma, neuroblastoma, endometrial cancer, and urothelial cell carcinoma.

In some aspects, the present disclosure includes the following embodiments (“Items”):
Item 1. An antibody-drug conjugate of Formula (1):

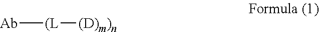

or a pharmaceutically acceptable salt thereof,
wherein Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6,
wherein D is an antitumor compound which is conjugated to the anti-BCAM antibody via the linker,
wherein n is 1 to 10,
wherein m is 1 to 5, and
wherein L is a linker represented by Formula (2):

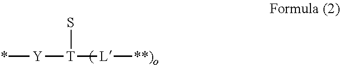

wherein Y is a divalent group comprising one or more atoms selected from C, N, O, P and S,
wherein T is a (1+o)- or (2+o)-valent connecting group,
wherein S is an atom or group that is optionally present to saturate a free valency of T,
wherein L′ is a linker capable of being cleaved by Cathepsin B,
wherein o is an integer of 1 to 5,
wherein * indicates covalent attachment to the anti-BCAM antibody (Ab), and
wherein ** indicates covalent attachment to one or more antitumor compounds (D).
Item 2. The antibody-drug conjugate of item 1, wherein the heavy chain variable region comprises a sequence of SEQ ID NO: 7, and the light chain variable region comprises a sequence of SEQ ID NO: 8.
Item 3. The antibody-drug conjugate of item 1 or 2, wherein n is 3 to 8 and m is 1.
Item 4. The antibody-drug conjugate of any of items 1 to 3, wherein a drug (the antitumor compound) to antibody (the anti-BCAM antibody) ratio (DAR) is from about 3 to about 8.
Item 5. The antibody-drug conjugate of item 4, wherein the DAR is about 4.
Item 6. The antibody-drug conjugate of any of items 1 to 5, wherein the antigen binding fragment thereof is an antibody fragment selected from the group consisting of a Fab fragment, a Fab′ fragment, a Fab′-SH, a Fv fragment, a scFv fragment, a F(ab′)₂ fragment, a VL fragment, a VH fragment, a ScFv-Fc fragment, and a (ScFv)₂-Fc fragment, a diabody, a linear antibody, a fragment produced by a Fab expression library, an anti-idiotypic (anti-Id) antibody, a complementary determining region (CDR), and an epitope-binding fragment.
Item 7. The antibody-drug conjugate of any of items 1 to 6, wherein the anti-BCAM antibody is a chimeric antibody, a humanized antibody, or a human antibody.
Item 8. The antibody-drug conjugate of any of items 1 to 7, wherein the linker is covalently attached to the antibody via the side chain of a cysteine comprised in the antibody.
Item 9. The antibody-drug conjugate of any of items 1 to 8, wherein the linker capable of being cleaved by Cathepsin B (L′) is represented by Formula (3), or Formula (4):

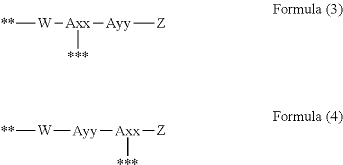

wherein Axx is a trifunctional amino acid, with the proviso that Axx in Formula (3) is not an amino acid in the (D) configuration,
wherein Ayy in Formulae (3) and (4) is an amino acid selected from Phe, Ala, Trp, Tyr, Phenylglycine (Phg), Met, Val, His, Lys, Arg, Citrulline (Cit), 2-amino-butyric acid (Abu), Ornithine (Orn), Ser, Thr, Leu and Ile, or Ayy in Formula (3) is an amino acid selected from homo-tyrosine (homo-Tyr), homo-phenylalanine (homo-Phe), betaphenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, with the proviso that Ayy in Formula (4) is not an amino acid in the (D) configuration,
wherein Z is a group covalently attached to the C-terminus of Ayy or Axx selected from —OH and —N(H)(R), wherein R represents a hydrogen atom, an alkyl group or a cycloalkyl group,
wherein W is a drug-carrying unit,
wherein ** indicates covalent attachment to one or more moieties D, and
wherein *** indicates covalent attachment to T,
if more than one linker L′ is present, each linker is independently selected from the aforementioned linkers of Formula (3) and Formula (4).
Item 10. The antibody-drug conjugate of item 9, wherein at least one, or both of Axx and Ayy is/are defined as follows:
(a) Axx in formula (3) or (4) is an amino acid selected from Glu, 2-amino-pimelic acid (Apa), 2-amino adipic acid (Aaa), 2,3-diamino-propionic acid (Dap), 2,4-diamino-butyric acid (Dab), Lys, Orn, Ser, amino-malonic acid (Ama), and homo-lysine (homo-Lys),
(b) Ayy in Formula (3) is an amino acid selected from Phe, homo-Phe, Ala, Trp, Phg, Leu, Val, Tyr, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24, or
(c) Ayy in Formula (4) is an amino acid selected from Phe, homo-Phe, Ala, Trp, Phg, Leu, Val, Tyr and Ser.
Item 11. The antibody-drug conjugate of item 9 or 10, wherein at least one, or both of Axx and Ayy is/are defined as follows:
(a) Axx in formula (3) or (4) is an amino acid selected from Dap, Dab, Lys, Orn and homo-Lys,
(b) Ayy in Formula (3) is an amino acid selected from Phe and Tyr, or
(c) Ayy in Formula (4) is an amino acid selected from Phe and Ser.
Item 12. The antibody-drug conjugate of any of items 9 to 11, wherein the drug-carrying unit (W) is a group represented by Formula (5):

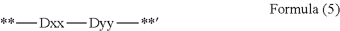

wherein Dxx is absent or an amino acid having a hydrophobic side chain,
wherein Dyy is absent, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein ** indicates covalent attachment to D, and
wherein **′ indicates covalent attachment to the N-terminus of Axx or Ayy.
Item 13. The antibody-drug conjugate of item 12, wherein at least one, e.g., one or two, of Dxx and Dyy is/are defined as follows:
(a) Dxx is an amino acid selected from Phe, Val, Tyr, homo-Phe and Ala,
(b) Dyy is absent, or an amino acid selected from Arg, Lys, Cit, Orn, Dap and Dab.
Item 14. The antibody-drug conjugate of any of items 9 to 11, wherein the drug-carrying unit (W) is a group represented by Formula (6), or Formula (7):

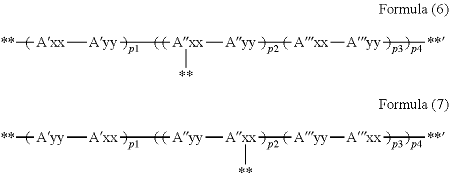

wherein A″xx is a trifunctional amino acid, with the proviso that A″xx in Formula (6) is not an amino acid in the (D) configuration,
wherein A′yy is an amino acid selected from Phe, Ala, Trp, Tyr, Phg, Met, Val, His, Lys, Arg, Cit, Abu and Orn, with the proviso that A′yy in Formula (7) is not an amino acid in the (D) configuration; if more than one A′yy are present, each A′yy is independently selected from the aforementioned amino acids,
wherein A″yy is an amino acid selected from Phe, Ala, Trp, Tyr, Phg, Met, Val, His, Lys, Arg, Cit, Abu and Orn, with the proviso that A″yy in Formula (7) is not an amino acid in the (D) configuration; if more than one A″yy are present, each A″yy is independently selected from the aforementioned amino acids,
wherein A′″yy is an amino acid selected from Phe, Ala, Trp, Tyr, Phg, Met, Val, His, Lys, Arg, Cit, Abu and Orn, with the proviso that A′″yy in Formula (7) is not an amino acid in the (D) configuration; if more than one A′″yy are present, each A′″yy is independently selected from the aforementioned amino acids,
wherein A′xx is an amino acid, with the proviso that A′xx in Formula (6) is not an amino acid in the (D) configuration,
wherein A′″xx is an amino acid, with the proviso that A′″xx in Formula (6) is not an amino acid in the (D) configuration,
wherein p1 is an integer of 0 to 3,
wherein p2 is 0 or 1,
wherein p3 is an integer of 0 to 3, with the proviso that if p2 is 0, p3 is not 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.
Item 15. The antibody-drug conjugate of item 14, wherein at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Lys, homo-Lys, Cit, Orn, Dap and Dab,
(c) A′″xx is an amino acid selected from Arg, Lys, homo-Lys, Cit, Orn, Dap and Dab,
(d) A′yy is an amino acid selected from Phe, Ala, Trp, Phg and Tyr,
(e) A″yy is an amino acid selected from Phe, Ala, Trp, Phg and Tyr,
(f) A′″yy is an amino acid selected from Phe, Ala, Trp, Phg and Tyr.
Item 16. The antibody-drug conjugate of any of items 9 to 11, wherein the drug-carrying unit is a group represented by Formula (8):

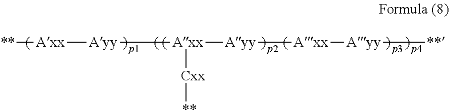

wherein A″xx is a trifunctional amino acid selected from Glu, α-amino adipic acid (Aaa), Dap, Ser, Thr, homo-serine (homo-Ser), homo-threonine (homo-Thr) and amino malonic acid (Ama), with the proviso that A″xx is not an amino acid in the (D) configuration,
wherein Cxx is a single covalent bond unless A″xx is Ama; if A″xx is Ama, Cxx is Pro or an N-methyl amino acid, the N-terminus of Cxx binds to a carboxyl end of Ama and the C-terminus of Cxx is covalently attached to one moiety D,
wherein A′yy, A″yy and A′″yy are each independently an amino acid selected from Phe, Ala, Trp, Tyr, Phg, Met, Val, His, Lys, Arg, Cit, Abu, Orn,
wherein A′xx and A′″xx are each independently an amino acid, with the proviso that A′xx and A′″xx are not an amino acid in the (D) configuration,
wherein p1 is 0 or 1,
wherein p2 is 0 or 1,
wherein p3 is an integer of 0 to 3, with the proviso that if p2 is 0, p3 is not 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.
Item 17. The antibody-drug conjugate of item 16, wherein at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Dap, Dab, Lys, Orn and homo-Lys,
(c) A′″xx is an amino acid selected from Arg, Lys, homo-Lys, Cit, Orn, Dap and Dab,
(d) A′yy is an amino acid selected from Phe, Ala, Trp, Phg and Tyr,
(e) A″yy is an amino acid selected from Phe, Ala, Trp, Phg and Tyr,
(f) A′″yy is an amino acid selected from Phe, Ala, Trp, Phg and Tyr.
Item 18. The antibody-drug conjugate of any of items 1 to 17, wherein the connecting group (T) is represented by Formula (9):

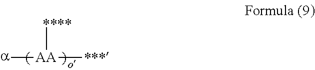

wherein each AA is independently a moiety comprising a trifunctional amino acid,
wherein α indicates covalent attachment of the N-terminus of AA, or the N-terminus of the first AA in case of o′ being 2 to 5, to Y,
wherein o′ is an integer of 1 to 5, with the proviso that o′ is 1 to 4 if another moiety L′ is attached to ***′,
if o′ is 1, the side chain of the trifunctional amino acid is covalently attached to S or L′, the C-terminus being covalently attached to the other moiety L′ or S, respectively,
if o′ is 2, 3, 4 or 5, **** indicates covalent attachment to L′, and ***′ indicates covalent attachment to S.
Item 19. The antibody-drug conjugate of item 18, wherein each AA is independently a moiety comprising an amino acid selected from N-ε-propargyloxycarbonyl-L-Lysine (Lys(Poc)), Asp, Glu, Orn, Lys, Dab and Dap.
Item 20. The antibody-drug conjugate of any of items 1 to 17, wherein the connecting group (T) is represented by Formula (10), or Formula (11):

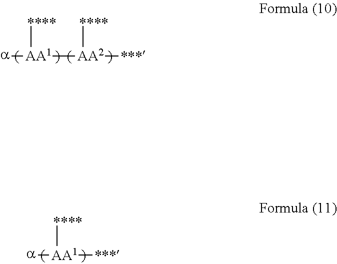

wherein each AA¹ and AA² is independently a moiety comprising a trifunctional amino acid,
wherein α indicates covalent attachment to Y,
wherein, in Formula (11), the side chain of the trifunctional amino acid is covalently attached to L′ or S, the C-terminus is covalently attached to the other moiety S or L′, respectively,
wherein, in Formula (10), **** indicates covalent attachment to L′, and ***′ indicates covalent attachment to S or L′.
Item 21. The antibody-drug conjugate of item 20, wherein each AA¹ and AA² is independently a moiety comprising an amino acid selected from Lys(Poc), Asp, Glu, Orn, Lys, Dab and Dap.
Item 22. The antibody-drug conjugate of any of items 1 to 17, wherein the connecting group (T) is represented by Formula (12), or Formula (13):

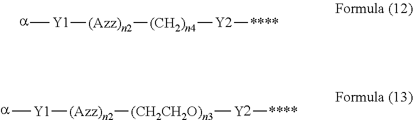

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to L′.
Item 23. An antibody-drug conjugate of Formula (1):

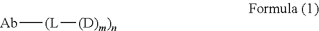

or a pharmaceutically acceptable salt thereof,
wherein Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6,
wherein D is an antitumor compound which is conjugated to the anti-BCAM antibody via the linker,
wherein n is 1 to 10,
wherein m is 1 to 5, and
wherein the linker (L) is represented by Formula (14), or Formula (15):

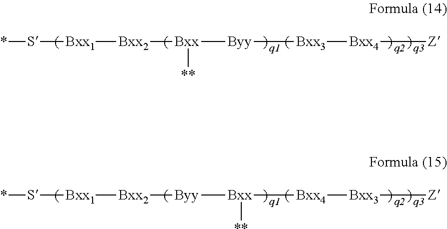

wherein Bxx in Formulae (14) and (15) is a trifunctional amino acid, with the proviso that Bxx in Formula (14) is not in the (D) configuration,
wherein Byy in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂, wherein R₂ is a hydrogen atom or a methyl group and n1 is an integer of 2 to 24, or Byy in Formula (14) is an amino acid selected from homo-Tyr, homo-Tyr(OR₁) wherein R₁ is as defined above, homo-Phe, beta-Phe and beta-homo-Phe; with the proviso that if q1*q3>1 and q2=0, only the C-terminal Byy in Formula (14) may be an amino acid selected from beta-Phe and beta-homo-Phe; with the proviso that Byy in Formula (15) is not in the (D) configuration,
wherein Bxx₁ in Formulae (14) and (15) is a single covalent bond or an amino acid having a hydrophobic or basic side chain,
wherein Bxx₂ in Formulae (14) and (15) is an amino acid having a hydrophobic or basic side chain,
wherein Bxx₃ in Formulae (14) and (15) is an amino acid, with the proviso that Bxx3 in Formula (14) is not in the (D) configuration,
wherein Bxx₄ in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂, wherein R₂ is a hydrogen atom or a methyl group and n1 is an integer of 2 to 24, or Bxx4 in Formula (14) is an amino acid selected from homo-Tyr, homo-Tyr(OR₁), homo-Phe, beta-Phe and beta-homo-Phe; with the proviso that if q2*q3>1, only the C-terminal Bxx4 in Formula (14) may be an amino acid selected from beta-Phe and beta-homo-Phe; with the proviso that Byy in Formula (15) is not in the (D) configuration,
or wherein the linker (L) is represented by Formula (16):

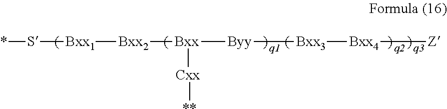

wherein Bxx in Formula (16) is a carboxylic amino acid, or a trifunctional amino acid selected from Dap, Dab, Ser, Thr, Lys, Orn, homo-Lys, homo-Ser and homo-Thr, with the proviso that Bxx is not in the (D) configuration,
wherein Cxx is a single covalent bond unless Bxx is Ama, if Bxx is Ama, Cxx is Pro or an N-methyl amino acid, the N-terminus of Cxx binds to a carboxyl group of Ama and the C-terminus of Cxx is covalently attached to one moiety D,
wherein Byy in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24; with the proviso that if q1*q3>1 and q2=0, only the C-terminal Byy may be an amino acid selected from beta-Phe and beta-homo-Phe,
wherein Bxx₁ in Formula (16) is a single covalent bond or an amino acid having a hydrophobic or basic side chain,
wherein Bxx₂ in Formula (16) is an amino acid having a hydrophobic or basic side chain,
wherein Bxx₃ in Formula (16) is an amino acid, with the proviso that Bxx3 is not in the (D) configuration,
wherein Bxx₄ in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24; with the proviso that if q2*q3>1, only the C-terminal Bxx4 may be an amino acid selected from beta-Phe and beta-homo-Phe,
wherein S′ is a divalent group comprising one or more atoms selected from C, N, O, P and S,
wherein Z′ is a group covalently attached to the C-terminus of Byy or Bxx4 in Formulae (14) and (16) or the C-terminus of Bxx or Bxx3 in Formula (15), which is selected from —OH and —N(H)(R), wherein R represents a hydrogen atom, an alkyl group or a cycloalkyl group,
wherein q1 is an integer of 0 to 5,
wherein q2 is an integer of 0 to 3, with the proviso that if q1 is 0, q2 is not 0,
wherein q3 is an integer of 1 to 5,
wherein q1, q2 and q3 are selected such that m in Formula (1) is an integer of 1 to 5,
wherein * indicates covalent attachment to the anti-BCAM antibody (Ab), and
wherein each ** indicates covalent attachment to one moiety D.
Item 24. The antibody-drug conjugate of item 23, wherein at least one, e.g., one, two, three, four, five or six, of Bxx, Byy, Bxx₁, Bxx₂, Bxx₃ and Bxx₄ is/are defined as follows:
(a) Bxx is an amino acid selected from Dap, Dab, Lys, Orn, Ser, Glu, Ama, Thr, Tyr, Aaa, homo-Ser and homo-Thr,
(b) Byy is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24,
(c) Bxx₁ is a single covalent bond, or an amino acid selected from Phe, homo-Phe, Phg, Val, Ser, Tyr, Ala, Leu and Ile,
(d) Bxx₂ is an amino acid selected from Arg, Lys, Cit, Val, Leu, Ser, Ala, Gly, His, Gln, Phg and Phe,
(e) Bxx₃ is an amino acid selected from Phe, homo-Phe, Phg, Val, Ser, Tyr, Ala, Leu and Ile,
(f) Bxx₄ is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24.
Item 25. The antibody-drug-conjugate of any of items 1 to 22, which is represented by Formula (17), or Formula (18):

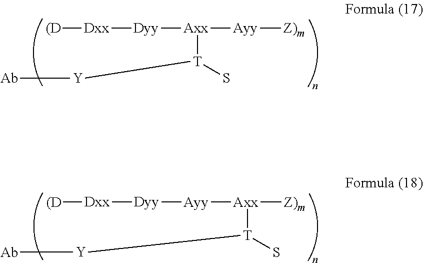

wherein Axx is an amino acid selected from Glu, Apa, Aaa, Dap, Dab, Lys, Orn, Ser, Ama and homo-Lys, with the proviso that Axx in Formula (17) is not in the (D) configuration,
wherein Ayy in Formula (17) is an amino acid selected from Phe, homo-Phe, Ala, Trp, Phg, Leu, Val, Tyr, homo-Tyr, Tyr(OR₁) and homo-Tyr(OR₁) wherein R₁ is —(CH₂ CH₂O)ₙ₁—R₂, wherein R₂ is a hydrogen atom or a methyl group and n1 is an integer of 2 to 24,
wherein Ayy in Formula (18) is an amino acid selected from Phe, homo-Phe, Ala, Trp, Phg, Leu, Val, Tyr and Ser, with the proviso that Ayy in Formula (18) is not in the (D) configuration,
wherein Dxx is a single covalent bond or an amino acid having a hydrophobic side chain,
wherein Dyy represents a single covalent bond, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein Y is a divalent group comprising one or more atoms selected from C, N, O, P and S,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of Ayy or Axx selected from —OH and —N(H)(R), wherein R represents a hydrogen atom, an alkyl group or a cycloalkyl group, and
wherein Ab, D, m and n are as defined in any of items 1 to 22.
Item 26. The antibody-drug-conjugate of item 25, wherein at least one, e.g., one, two, three, four, five, six, seven or eight, of Axx, Ayy, Dxx, Dyy, D, Z, m and T is/are defined as follows:
(a) Axx is an amino acid selected from Dap, Dab, Lys, Om and homo-Lys,
(b) Ayy in Formula (17) is an amino acid selected from Phe, homo-Phe, Tyr, homo-Tyr, Tyr(OR₁) and homo-Tyr(OR₁),
(c) Ayy in Formula (18) is an amino acid selected from Phe, home-Phe or Ser,
(d) Dxx is a moiety derived from an amino acid selected from Phe, Val, Tyr, homo-Phe and Ala,
(e) Dyy is a covalent bond or a moiety derived from an amino acid selected from Arg, Lys, Cit, Orn, Dap and Dab,
(f) D is an antitumor compound selected from auristatin F (AF), MMAF, exatecan, maytansine, DM1 and DM4,
(g) Z is —OH or —NH₂,
(h) T is represented by Formula (9′):

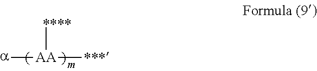

wherein each AA is independently a moiety comprising a trifunctional amino acid,
wherein α indicates covalent attachment to Y,
wherein m is 1 to 5,
if m is 1, the side chain of the trifunctional amino acid is covalently attached to S or Axx, the C-terminus being covalently attached to the other moiety S or Axx, respectively,
if m is 2, 3, 4 or 5, **** indicates covalent attachment to Axx, and ***′ indicates covalent attachment to S via the C-terminus of the chain of AA groups,
(i) m is 2 and T is represented by Formula (10′):

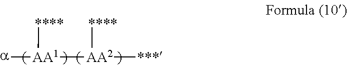

wherein each AA¹ and AA² is independently a moiety comprising a trifunctional amino acid,
wherein α indicates covalent attachment to Y,
wherein **** indicates covalent attachment to Axx, and ***′ indicates covalent attachment to S,

(j) m is 1 and T is represented by Formula (11′):

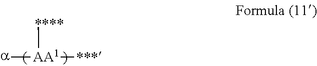

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(k) m is 1 and T is represented by Formula (12′), or Formula (13′):

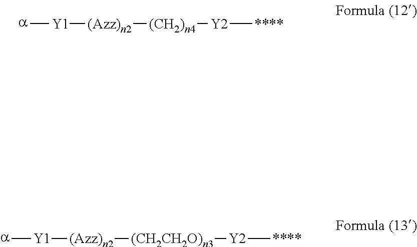

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.
Item 27. The antibody-drug-conjugate of item 25 or 26, wherein at least one, e.g., one, two, three, four, five, six, seven, eight or nine, of Axx, Ayy, Dxx, Dyy, D, Z, m and T is/are defined as follows:
(a) Axx is Lys,
(b) Ayy in Formula (17) is Tyr,
(c) Ayy in Formula (18) is Phe or Ser,
(d) Dxx is Phe or Val,
(e) Dyy is Arg or Cit,
(f) D is an antitumor compound selected from AF, MMAF, exatecan, maytansine, DM1 and DM4,
(g) Z is —OH or —NH2,
(h) m is 1 and T is represented by Formula (11′):

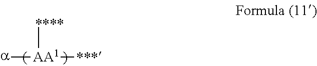

wherein AA¹ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(i) m is 1 and T is represented by Formula (12′), or Formula (13′):

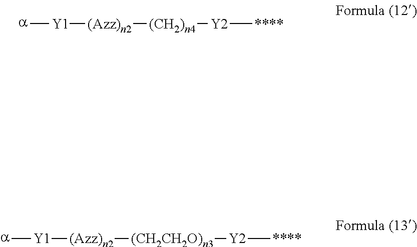

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.
Item 28. The antibody-drug-conjugate of any of items 25 to 27, wherein, in Formula (17), each Dxx-Dyy-Axx-Ayy is independently selected from Arg-Lys-Phe wherein Dxx is a covalent bond, Arg-Lys-homoPhe wherein Dxx is a covalent bond, Arg-Lys-Tyr wherein Dxx is a covalent bond, Cit-Lys-Phe wherein Dxx is a covalent bond, Cit-Lys-Tyr wherein Dxx is a covalent bond, Arg-Lys-homoTyr wherein Dxx is a covalent bond, Cit-Lys-homoTyr wherein Dxx is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr, and
wherein, in Formula (18), each Dxx-Dyy-Ayy-Axx is independently selected from Arg-Phe-Lys wherein Dxx is a covalent bond, Arg-Ser-Lys wherein Dxx is a covalent bond, Cit-Phe-Lys wherein Dxx is a covalent bond, Cit-Ser-Lys wherein Dxx is a covalent bond, Cit-homoPhe-Lys wherein Dxx is a covalent bond, Phe-Cit-Phe-Lys, homoPhe-Cit-Phe-Lys, and Phe-Arg-Phe-Lys.
Item 29. The antibody-drug-conjugate of any of items 25 to 28, wherein at least one, e.g., one, two, three or four, of D, Z, m and T is/are defined as follows:
(a) D is an antitumor compound selected from AF, MMAF, exatecan, maytansine, DM1 and DM4,
(b) Z is —OH or —NH₂,
(c) m is 1 and T is represented by Formula (11′):

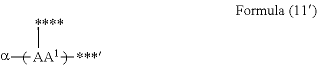

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(d) m is 1 and T is represented by Formula (12′), or Formula (13′):

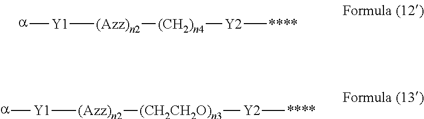

wherein each Azz is independently a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.
Item 30. The antibody-drug-conjugate of any of items 1 to 22 and 25 to 29, which is represented by one of the following formulae:

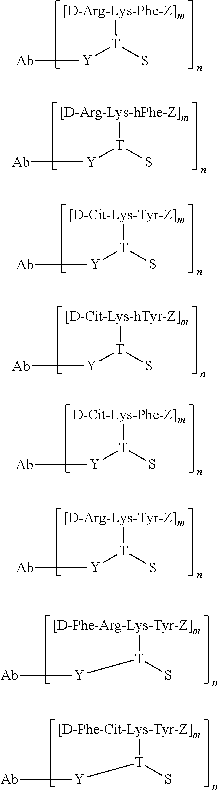

with the proviso that in the above formulae, Lys is not in the (D) configuration,
wherein Y is a divalent group comprising one or more atoms selected from C, N, O, P and S,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of an amino acid selected from —OH and —N(H)(R), wherein R represents a hydrogen atom, an alkyl group or a cycloalkyl group, and
wherein Ab, D, m and n are as defined in any of items 1 to 22.
Item 31. The antibody-drug-conjugate of item 30, wherein at least one, e.g., one, two, three or four, of D, Z, m and T is/are defined as follows:
(a) D is an antitumor compound selected from AF, MMAF, exatecan, maytansine, DM1 and DM4,
(b) Z is —OH or —NH₂,
(c) m is 2 and T is represented by Formula (10′):

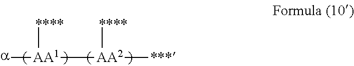

wherein each AA¹ and AA² is independently a moiety comprising a trifunctional amino acid,
wherein α indicates covalent attachment to Y,
wherein **** indicates covalent attachment to Lys, and ***′ indicates covalent attachment to S,
(d) m is 1 and T is represented by Formula (11′):

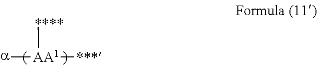

wherein AA¹ is a moiety comprising a trifunctional amino acid,
wherein α indicates covalent attachment to Y,
wherein the side chain of the trifunctional amino acid is covalently attached to Lys or S, the C-terminus is covalently attached to the other moiety S or Lys, respectively,
(e) m is 1 and T is represented by Formula (12′), or Formula (13′):

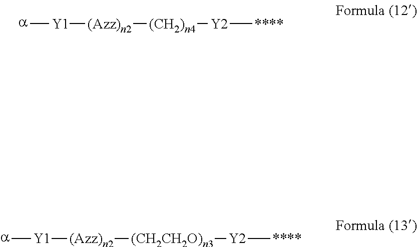

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Lys.
Item 32. The antibody-drug-conjugate of item 30 or 31, wherein D, Z, m and T are defined as follows:
(a) D is DM1,
(b) Z is —OH or —NH₂,
(c) m is 1 and T is represented by Formula (13′):

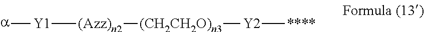

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Lys.
Item 33. The antibody-drug-conjugate of any of items 30 to 32, wherein D, Z, m and T is/are defined as follows:
(a) D is AF,
(b) Z is —OH or —NH₂,
(c) m is 1 and T is represented by Formula (13′):

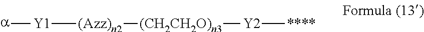

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Lys.
Item 34. The antibody-drug-conjugate of any of items 1 to 22, which is represented by Formula (19), Formula (20), Formula (21), or Formula (22):

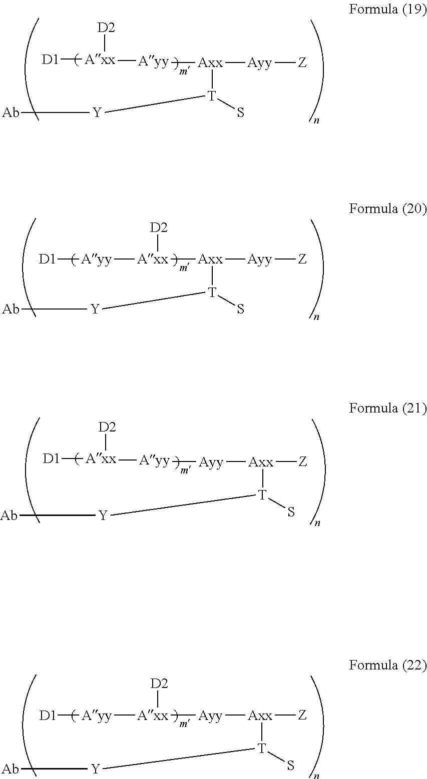

wherein Axx is a trifunctional amino acid, with the proviso that Axx in Formula (19) and Formula (20) is not an amino acid in the (D) configuration,
wherein Ayy is an amino acid selected from Phe, Ala, Trp, Tyr, Phg, Met, Val, His, Lys, Arg, Cit, Abu, Orn, Ser, Thr, Leu and Ile, or Ayy in Formula (19) and Formula (20) is an amino acid selected from homo-Tyr, homo-Phe, beta-Phe and beta-homo-Phe, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, with the proviso that Ayy in Formula (21) and Formula (22) is not an amino acid in the (D) configuration,
wherein each A″yy is independently an amino acid selected from Phe, Ala, Trp, Tyr, Phg, Met, Val, His, Lys, Arg, Cit, Abu, Orn, with the proviso that A″yy in Formula (20) and Formula (22) is not an amino acid in the (D) configuration,
wherein Y is a divalent group comprising one or more atoms selected from C, N, O, P and S,
wherein T is a tri-valent connecting group; if S is absent, T is a divalent connecting group,
wherein S is an atom or group that is optionally present to saturate a free valency of T,
wherein Z represents a group covalently bonded to the C-terminus of Ayy in Formula (19) and Formula (20) or to the C-terminus of Axx in Formula (21) or Formula (22), which is selected from —OH and —N(H)(R), wherein R represents a hydrogen atom, an alkyl group or a cycloalkyl group,
wherein D1 is an antitumor compound,
wherein m′ is (m−1), m being as defined in claim 1, with the proviso that m′ is not 0,
if m′ is 1, A″xx is a trifunctional amino acid with the proviso that A″xx in Formula (19) and Formula (21) is not an amino acid in the (D) configuration, and D2 is an antitumor compound,
if m′ is more than 1, each D2 is independently selected from a hydrogen atom and an antitumor compound, wherein multiple moieties D2 can be the same or different with the proviso that at least one D2 is not a hydrogen atom; if D2 is a hydrogen atom, A″xx is an amino acid with the proviso that A″xx in Formula (19) and Formula (21) is not an amino acid in the (D) configuration; if D2 is an antitumor compound, A″xx is a trifunctional amino acid with the proviso that A″xx in Formula (19) and Formula (21) is not an amino acid in the (D) configuration,
wherein Ab and n are as defined in any of items 1 to 22.
Item 35. The antibody-drug-conjugate of item 34, wherein at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Om and homo-Lys,
(b) Ayy in Formula (19) and Formula (20) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is independently an antitumor compound selected from AF, MMAF, exatecan, maytansine, DM1 and DM4,
(f) Z is —OH or —NH₂,
(g) m is 1 and T is represented by Formula (11′):

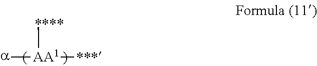

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(h) m is 1 and T is represented by Formula (12′), or Formula (13′):

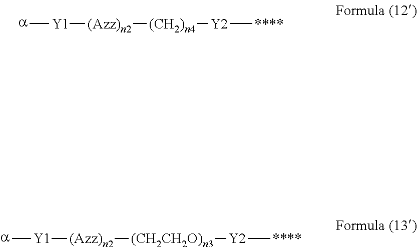

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.
Item 36. The antibody-drug-conjugate of item 34 or 35, wherein at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Om and homo-Lys,
(b) Ayy in Formula (17) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is AF,
(f) Z is —OH or —NH₂,
(g) m′ is 1 and T is represented by Formula (13′):

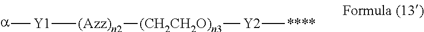

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Axx.
Item 37. The antibody-drug-conjugate of any of items to 36, wherein the antitumor compound (D) is selected from DNA-alkylating agents, topoisomerase inhibitors, RNA-polymerase II inhibitors, DNA-cleaving agents, antimitotic agents or microtubule disruptors, anti-metabolites, Kinesin spindle protein inhibitors, kinase inhibitors, nicotinamide phosphoribosyl transferase inhibitors, matrix metallopeptidase 9 inhibitors, phosphatase inhibitors, or radioisotopes and/or pharmaceutically acceptable salts thereof; if more than one D is present, each D is independently selected from the aforementioned compounds.
Item 38. The antibody-drug-conjugate of any of items 1 to 37, wherein the antitumor compound D is selected from amanitin, duocarmycin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof; if more than one D is present, each D is independently selected from the aforementioned compounds.
Item 39. The antibody-drug-conjugate of any of items 1 to 22 and 25 to 38, which is selected from the following compounds:

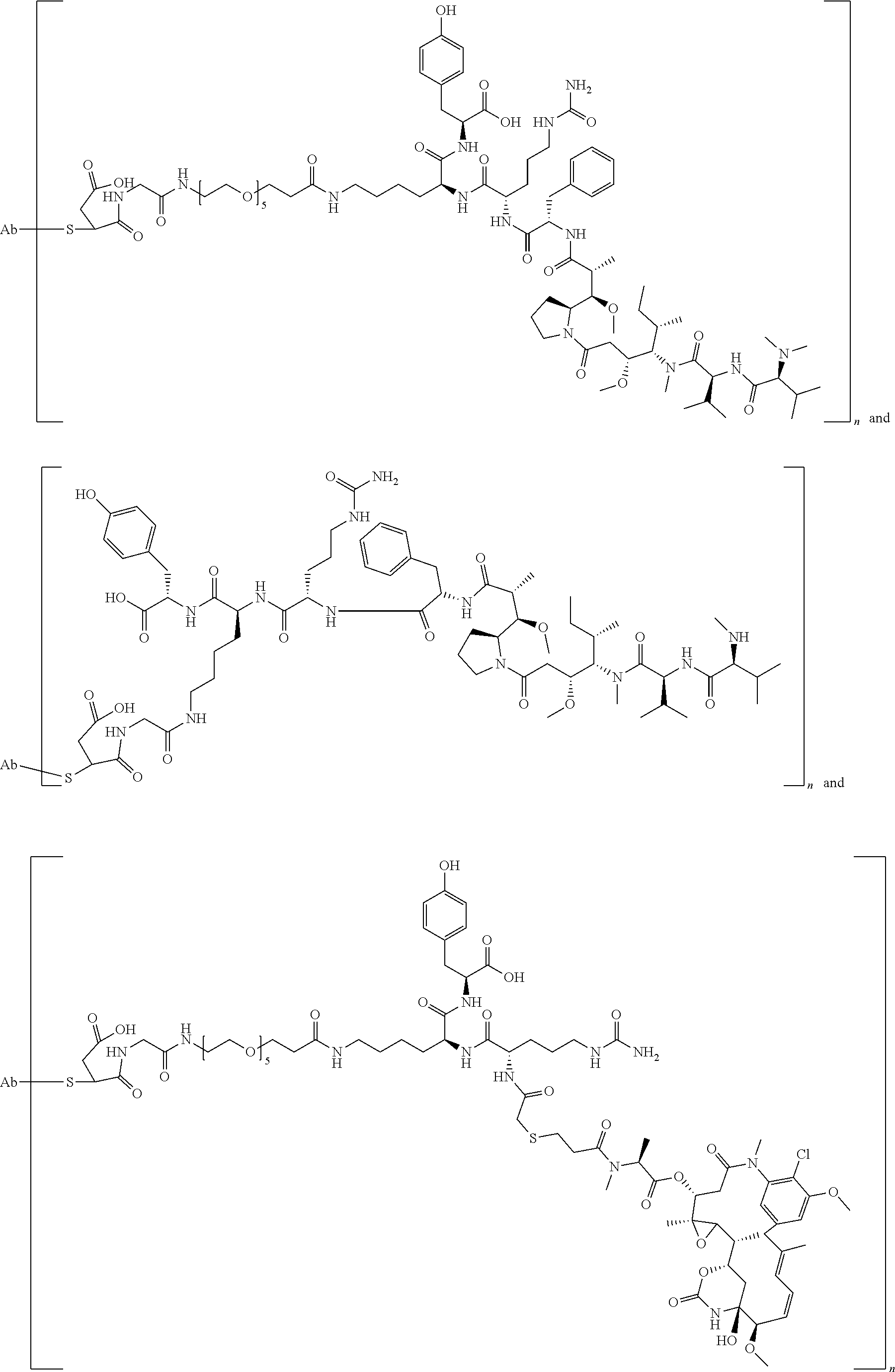

wherein each Ab and n are as defined in any of items 1 to 3 and/or 6 to 8.
Item 40. A pharmaceutical composition comprising the antibody-drug conjugate of any of items 1 to 39 or a pharmaceutically acceptable salt thereof, and a pharmaceutically acceptable carrier.
Item 41. The pharmaceutical composition of item 40, wherein the pharmaceutical composition is for use in treating or imaging cancer.
Item 42. The pharmaceutical composition for use of item 41, wherein the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.
Item 43. A method for treating cancer, the method comprising administering an effective amount of the antibody-drug conjugate of any of items 1 to 39 or a pharmaceutically acceptable salt thereof to a subject in need thereof.
Item 44. The method of item 43, wherein the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

### Solution to Problem

### Definitions

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof such as “contain” denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language. The term “comprise” and linguistic variations thereof should also be understood as disclosing, as a more restricted embodiment, the term “consisting of” as well, such that no additional unmentioned elements may be present, as long as this is technically meaningful.

Where the present description refers to “preferred” embodiments/features, combinations of these “preferred” embodiments/features shall also be deemed as disclosed as long as this combination of “preferred” embodiments/features is technically meaningful.

The term “antibody-drug conjugate,” as used herein, refers to the linkage of an antibody or an antigen binding fragment thereof with another antitumor compound, such as a chemotherapeutic agent, a toxin, an immunotherapeutic agent, an imaging probe, and the like. The linkage can be covalent bonds.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies as well as single chain antibodies and humanized antibodies.

The term “monoclonal antibody” as used herein refers to an antibody obtained from a monoclonal cell line that produce substantially homogeneous antibodies, i.e., the individual antibodies produced are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

The term “polyclonal antibody” as used herein refers to a mixture of antibodies that are secreted by different B cell lineages. These antibodies are actually a collection of immunoglobulin molecules that react against a specific antigen.

The term “chimeric antibody,” as used herein refers to antibodies in which the variable region sequences are derived from one species and the constant region sequences are derived from another species, such as an antibody in which the variable region sequences are derived from a rat antibody and the constant region sequences are derived from a human antibody.

The term “humanized antibody,” as used herein, refers to forms of non-human (e.g. rat) antibodies that are chimeric immunoglobulins, immunoglobulin chains, or fragments thereof that contain minimal sequence derived from non-human immunoglobulin. Humanized antibodies may be human immunoglobulins in which residues from a complementary determining region (CDR) of the recipient are replaced by residues from a CDR of a non-human species (donor antibody) such as mouse, rat, or rabbit having the desired specificity, affinity, and capacity.

The term “human antibody,” as used herein, refers to antibodies having variable regions in which both the framework and CDR regions are derived from sequences of human origin. If the antibody contains a constant region, the constant region also is derived from such human sequences, e.g., human germline sequences, or mutated versions of human germline sequences or antibody containing consensus framework sequences derived from human framework sequences analysis.

The term “complementarity-determining regions (CDRs),” as used herein, refer to the hypervariable regions of a light chain variable region (VL) and a heavy chain variable region (VH). The CDRs are the target protein-binding site of the antibody chains that harbors specificity for such target protein. There are three CDRs (CDR1-3, numbered sequentially from the N-terminus) in each human VL or VH, constituting about 15-20% of the variable domains. For instance, the term “CDRH1,” as used herein refers to the first CDR of the heavy chain variable region, and the term “CDRL1,” as used herein refers to the first CDR of the light chain variable region. The CDRs are structurally complementary to the epitope of the target protein and are thus directly responsible for the binding specificity. The remaining stretches of the VL or VH, the so-called framework regions, exhibit less variation in amino acid sequence.

The light and heavy chains are divided into regions of structural and functional homology. The terms “constant” and “variable” are used functionally. The variable domains of both the light (VL) and heavy (VH) chain portions determine antigen recognition and specificity. The constant domains of the light chain (CL) and the heavy chain (CH1, CH2 or CH3) confer important biological properties such as secretion, transplacental mobility, Fc receptor binding, complement binding, and the like. The more highly conserved portions of variable regions are called the framework regions (FRs). The variable regions of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity.

The term “antigen binding fragment thereof,” as used herein, refers to a portion of a full length antibody, generally the antigen binding or variable region thereof. Examples of the antibody fragment include a Fab fragment, a Fab′ fragment, a Fab′-SH, a Fv fragment, a scFv fragment, a F(ab′)₂ fragment, a VL fragment, a VH fragment, a ScFv-Fc fragment, a (ScFv)₂-Fc fragment, diabodies, linear antibodies, fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules, and multispecific antibodies formed from antibody fragments.

The term “anti-basal cell adhesion molecule (BCAM) antibody,” as used herein, refers to an antibody specifically binds to basal cell adhesion molecule (BCAM). By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “pharmaceutically acceptable salt,” as used herein, refers to pharmaceutically acceptable organic or inorganic salts of disclosed compounds such as an antibody-drug conjugate or an antitumor compound. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. Lists of suitable salts can be found in Remington's Pharmaceutical Sciences, 17^{th} ed., Mack Publishing Company, Easton, PA, 1985, page 1418, S. M. Berge, L. M. Bighley, and D. C. Monkhouse, “Pharmaceutical Salts” J. Pharm. Sci. 1977, 66(1), 1-19; P. H. Stahl and C. G. Wermuth, editors, Handbook of Pharmaceutical Salts: Properties, Selection and Use, Weinheim/Zurich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. Unless the context dictates otherwise, all references to compounds of the disclosure (or invention) are to be understood also as references to pharmaceutically acceptable salts of the respective compounds.

The term “linker,” as used herein, refers to a moiety that connects two moieties of the antibody and the antitumor compound by covalent connections. In some instances, the term linker “linker” as used herein may refer to a moiety comprising a cleavable element, as well as further elements such as a connecting group, a group comprising one or more solubilizing groups, etc. In some other instances, the term “linker” as used herein may refer to a specifically defined element, such as a linker “capable of being cleaved by Cathepsin B” (as described further below).

The term “cleavable” as used herein, refers to a linker that connects two moieties of the antibody and the antitumor compound by covalent connections, but breaks down to sever the covalent connection between the moieties under physiologically relevant conditions. Cleavage generally releases the antitumor compound from the antibody. The term “cleavable” as used herein, refers to a linker that is not especially susceptible to breaking down under physiological conditions. Such a linker is sufficiently resistant to degradation to keep the antitumor compound connected to the antibody or antigen binding fragment until the antibody or antigen binding fragment is itself at least partially degraded.

The expression “capable of being cleaved by Cathepsin B” characterizes any compound (or moiety that may be incorporated into a compound), which is cleaved when being contacted with Cathepsin B (Cat B) under suitable conditions e.g., as set out in Section 2.3.5 below or in WO2019096867 (cf. Section 11.3.1). In preferred embodiments, said cleavage is (a) fast and/or (b) cleavage is via the exopeptidase activity of Cat B. Said embodiment (b), relating to a compound or moiety that is “capable of being cleaved by the exopeptidase activity of Cat B”, is defined in more detail in the next paragraph. The above-mentioned “fast” cleavage of embodiment (a) typically means for a compound of interest that the corresponding unconjugated compound (i.e., compound not having an antibody and being quenched at the conjugation group, for example with cysteine being covalently attached to a maleimide conjugation group) has a cleavage rate T_{1/2} of 25 min or less, preferably 20 min or less, more preferably 18 min or less, even more preferably 16 min or less and most preferably 14 min or less in the conditions of the Cat B-cleavage assay described in Section 2.3.5. below or in WO2019096867 (cf. Section 11.3.1). There is no particular lower limit. However, it is realistic to expect cleavage rates T_{1/2} of 0.2 min or more, typically 0.5 min or more, and even more typically 1 min or more, such as 2 min or more in the conditions of the Cat B-cleavage assay described in Section 2.3.5. below or in WO2019096867 (cf. Section 11.3.1).

The expression “capable of being cleaved by the exopeptidase activity of Cat B” as used herein indicates that the respective moiety of the compound, in particular the linker, e.g., C-terminal peptide unit, can be specifically recognized and cleaved by the exopeptidase (i.e., carboxydipeptidase) of Cathepsin B. Said cleavage gives rise to the rapid release of the drug (or a modified drug having group or moiety that remains attached thereto after cleavage by Cathepsin B, “intra-drug”) into the target cell. The cleavage of a linker, e.g., a C-terminal peptide unit, via the exopeptidase activity of Cat B can be assessed by the in vitro enzymatic cleavage assay using recombinant human Cat B and UHPLC-MS/MS analysis as described further below. Considering that exopeptidase activity of Cat B is typically associated with higher cleavage rates compared to endopeptidase activity of Cat B, in some aspects, the expression “compound capable of being cleaved by the exopeptidase activity of Cat B” may be confirmed by confirming a high Cat B cleavage rate. According to this aspect, a “compound capable of being cleaved by the exopeptidase activity of Cat B” refers to a compound for which the following criterion is fulfilled: the corresponding unconjugated compound (i.e. compound not having an antibody and being quenched at the conjugation group, for example with cysteine being covalently attached to a maleimide conjugation group) has a cleavage rate T½ of 25 min or less, preferably 20 min or less, more preferably 18 min or less, even more preferably 16 min or less and most preferably 14 min or less in the conditions of the Cat B-cleavage assay described in Section 2.3.5. below or in WO2019096867 (cf. Section 11.3.1). There is no particular lower limit. However, it is realistic to expect cleavage rates T½ of 0.2 min or more, typically 0.5 min or more, and even more typically 1 min or more, such as 2 min or more in the conditions of the Cat B-cleavage assay described in Section 2.3.5. below or in WO2019096867 (cf. Section 11.3.1).

The term “peptide” as used herein refers to a compound comprising a continuous sequence of at least two amino acids linked to each other via peptide linkages. The terms “dipeptide”, “tripeptide” and “tetrapeptide” respectively refer to a compound comprising a continuous sequence of two, three and four amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues. For instance, the modified linkage may be an ester, a thioester, a carbamide, a thiocarbamide or a triazole linkage. Preferably, the amino acids forming the continuous peptide sequence are linked to each other via backbone amide bonds. The peptide may be linear or branched. In preferred aspects, the peptide is a linear di-, tri-, tetra-peptide, more preferably a linear tri- or tetra-peptide.

The term “amino acid” as used herein refers to a compound that contains or is derived from a compound containing at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. The term “amino acid” encompasses both naturally occurring amino acids such as the naturally occurring proteinogenic amino acids, as well as synthetic amino acids that are not found in nature. In the following, a reference to amino acids may be made by means of the 3-letter amino acid code (Arg, Phe, Ala, Cys, Gly, Gln, etc.), or by means of the 1-letter amino acid code (R, F, A, C, G, Q, etc.). Unless specified otherwise, reference to an amino acid by means of the 3-letter amino acid code refers to the corresponding (L)- or (D)-amino acid.

Herein, amino acid sequences are written from the N-terminus to the C-terminus (left to right). Unless specified otherwise or dictated otherwise by the context, all connections between adjacent amino acid groups are formed by peptide (amide) bonds.

The expression “amino acid in the (D) configuration” as used herein refers to the (D)-isomer of any naturally occurring or synthetic amino acid. This applies to α-amino acids as well as to β- and g-amino acids. The expression “amino acid in the (D) configuration” as used herein is not meant to encompass non-chiral amino acids such as glycine or other non-chiral amino acids such as amino-isobutyric acid.

The expression “side chain of an amino acid” as used herein may refer to a moiety attached to the α-carbon of an amino acid. For example, the side chain of Ala is methyl, the side chain of Phe is phenylmethyl, the side chain of Cys is thiomethyl, the side chain of Tyr is 4-hydroxyphenylmethyl, etc. Both naturally occurring side chains and non-naturally occurring side chains are included by this definition.

The term “functional group” refers to a group that is capable of bonding to another functional group by forming at least one covalent bond without need for breaking any C—C or C—H covalent bonds.

The term “trifunctional” as used herein refers to a compound or moiety having three functional groups that can form or have formed three covalent bonds to adjacent moieties. Thus, the term “trifunctional amino acid” refers to a compound that contains or is derived from a compound containing at least an amino group, an acid group (e.g., a carboxyl group) and another functional group such as an amino group or a carboxyl group. Non-limiting examples of trifunctional amino acids include Ser, Cys, Tyr, NF-propargyloxycarbonyl-L-Lysine (Lys(Poc)), Asp, Glu, Orn, Lys, Dab and Dap.

The term “C-terminal” as used herein refers to the C-terminal end of the amino acid (peptide) chain. Binding to the “C-terminus” means that a covalent bond is formed between the acid group in the main chain (backbone) of the amino acid residue and the binding partner. For instance, binding of group “X” to the C-terminus of amino acid residue “Axx” yields an ester or amide-type structural element *—C(O)—X, wherein the carbonyl group is derived from the acid group of Axx and (*) indicates attachment to main chain. The term “C-terminal peptide unit” is used herein to characterize a peptide sequence of 2, 3 or 4 amino acids wherein the C-terminal amino acid forms the C-terminus of the peptide sequence.

The term “N-terminal” as used herein refers to the N-terminal end of the amino acid (peptide) chain. Binding to the “N-terminus” means that a covalent bond is formed between the amino group in the main chain (backbone) of the N-terminal amino acid residue and the binding partner (which replaces one hydrogen atom). For instance, binding of group “X” to the N-terminus of amino acid residue “Axx” yields a structural element X—NH—*, wherein the amino group is derived from Axx and (*) indicates attachment to main chain.

The term “hydrophobic” is used herein to characterize compounds, groups, or moieties, which lack affinity for water. For instance, the term “amino acid with hydrophobic side chain” is used to characterize amino acids with a hydrophobic or partially hydrophobic aliphatic side chain or amino acids with aromatic side chain such as Phe, Leu, Ile, Val, Tyr, Trp, Ala. Of course, any other amino acid exhibiting the same or a higher degree of hydrophobicity should also be treated as hydrophobic in the sense of the present disclosure. A comparison of the degree of hydrophobicity can be done by determining the n-octanol/water partition coefficient (at 25° C. and pH 7): if the ratio of concentrations in n-octanol/water for another amino acid is equal or higher than that of one or more of the amino acids Phe, Leu, Ile, Val, Tyr, Trp, Ala, such other amino acid is to be treated as a hydrophobic amino acid.

The term “amino acid with a basic side chain” is used herein to characterize natural or unnatural amino acids wherein the side chain contains one or more ionizable groups having a pKa value equal to or greater than 6. Examples of natural amino acids with a basic side chain include Arg (guanidino group, pKa=12.5), Lys (amino group, pKa=10.5), His (imidazole group, pKa=6). Examples of unnatural amino acids with a basic side chain include citrulline (Cit), ornithine (Orn), 2,3-diamino-propionic acid (Dap), 2,4-diamino-butyric acid (Dab).

The term “alkyl group” as used herein refers to a linear (straight chain) or branched, saturated or unsaturated hydrocarbon group having from 1 to 20 carbon atoms, preferably from 1 to 5 carbon atoms. Examples of alkyl groups include, but are not limited to, -methyl, -ethyl, -n-propyl, -n-butyl, -n-pentyl, -n-hexyl, -n-heptyl, -n-octyl, -isopropyl, -sec-butyl, -isobutyl, -tert-butyl, -isopentyl, -vinyl, -allyl, -1-butenyl, -2-butenyl, -1-pentenyl, -2-pentenyl, -3-methyl-1-butenyl, -2-methyl-2-butenyl, -2,3-dimethyl-2-butenyl, -1-hexyl, 2-hexyl, -3-hexyl, -propynyl, -1-butynyl, -2-butynyl, -1-pentynyl, -2-pentynyl and -3-methyl-1 butynyl. More preferably, the alkyl group is a methyl or an ethyl group.

The term “cycloalkyl group” as used herein refers to a substituted or unsubstituted, cyclic hydrocarbon group having from 3 to 20 carbon atoms, preferably from 5 to 8 carbon atoms. The cycloalkyl group may consist of a single ring, but it may also be formed by two or more condensed rings. Examples of cycloalkyl groups include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. More preferably, the cycloalkyl group is a cyclopentyl or cyclohexyl group.

The term “divalent maleimide derivative” (or, e.g., “divalent group derived from a compound selected from maleimide . . . ”) as used herein refers to a divalent moiety derived from maleimide, in which the double bond is hydrogenated, and two hydrogen atoms are replaced by two covalent bonds allowing attachment to adjacent moieties. For example, the divalent maleimide derivative may have one of the following structures (wherein R and R′ represent adjacent moieties to which said maleimide derivative is attached):

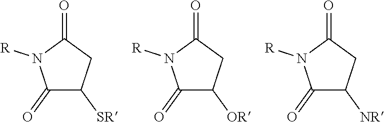

Said moiety contains a chiral carbon atom (i.e., the atom carrying the sulfur atom). Unless specified otherwise, references to a divalent maleimide derivative are to be understood as references to the pure stereoisomers as well as any mixture thereof and especially the racemic mixture thereof.

The term “divalent maleimide derivative” or “divalent group derived from a compound selected from maleimides” is further to be understood as encompassing any derivative of maleimide (as described above) additionally being substituted at other positions than positions 2 and 3, as well as opened hydrolyzed maleimide derivatives.

A divalent maleimide-type disulfide bridge (e.g., a divalent group of formula —S—X²—S—/—S—X³—S— wherein X²/X³ represents a divalent group derived from maleimide) can be obtained by side-chain-to-side-chain cyclization in the presence of e.g. 2,3-dibromomaleimide or another suitable reagent as described by Kuan et al. in Chem. Eur. J. 2016, 22, 17112-17129.

In the context of the present disclosure, an “opened hydrolyzed maleimide derivative” refers to a divalent moiety derived from maleimide wherein the maleimide ring has been opened by hydrolysis. For instance, the hydrolysis of the divalent maleimide derivative R—X—S—R′ (wherein X represents an unsubstituted divalent group derived from maleimide and R/R′ represent adjacent groups or moieties; in X the double bond of maleimide is no longer present) leads to an opened hydrolyzed maleimide derivative of formula R—NH—C(═O)—CH(S—R′)—CH₂—COOH, or R—NH—C(═O)—CH₂—CH(S—R′)—COOH, or a mixture thereof. The ring hydrolysis can be performed, for example, under basic conditions. The following conditions are especially suitable: at the end of a conjugation reaction (e.g., after the reaction of a maleimide moiety with the side chain of a cysteine residue contained in a BCAM antibody or an antigen binding fragment thereof), pH is adjusted to pH 8 by adding 10×pH 8 DPPS (0.2 to 0.5 reaction volume) and excess reactive drug linker and reducing agent (TCEP) are removed via gel filtration using suitable columns for gel filtration (e.g., PF column, elution with pH 8 buffer), the eluent is then stirred overnight for 16 h to complete the opening before final buffer exchange with DPBS into an Amicon concentrating unit. Unless specified otherwise, any reference to an “opened hydrolyzed maleimide derivative” is to be understood as a reference to one of these structures alone or any mixture of these structures. Moreover, the carbon carrying the sulfur atom is chiral. Unless specified otherwise, any reference to an “opened hydrolyzed maleimide derivative” is to be understood as a reference to the pure stereoisomers as well as any mixture thereof and especially the racemic mixture thereof.

Furthermore, in the context of the invention, a “maleimide attachment” refers to a divalent moiety derived from maleimide as described above which contains two covalent bonds allowing attachment to adjacent groups or moieties. For example, in the maleimide derivative of the formula R—X—S—R′, where R/R′ represent adjacent groups or moieties, X represents the maleimide attachment (a divalent group derived from maleimide in which the double bond of maleimide is no longer present). Thus, the term “maleimide attachment” is synonymous with “maleimide derivative attachment”.

Similarly, in the context of the invention, an “opened hydrolyzed attachment” refers to a divalent moiety derived from maleimide as described above which contains two covalent bonds allowing attachment to adjacent groups or moieties. For example, in the opened hydrolyzed maleimide derivative of the formula R—X—S—R′, where R/R′ represent adjacent groups or moieties, X represents the opened hydrolyzed maleimide attachment. Thus, the term “opened hydrolyzed attachment” is synonymous with “opened hydrolyzed derivative attachment”.

References to “a divalent group derived from a compound selected from . . . triazoles” are meant to characterize divalent groups resulting from a 3+2 cycloaddition of an alkyne and an azide. Such divalent groups are typically characterized by the following structures:

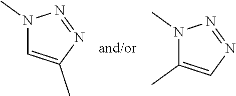

wherein the single bonds characterize attachment to adjacent groups, such that there is no particular restriction as to which adjacent group is bonded to the nitrogen atom and which adjacent group is bonded to the carbon atom. In the context of group Y, the divalent group may be formed by reacting an alkyne-containing group attached to V with an azide-containing group attached to T or vice versa in the presence or absence of a metal catalyst (as described, e.g., by Becer et al. in “Click Chemistry beyond Metal-Catalysed Cycloaddition” Angewandte Chemie Int. Ed. 2009, 48(27), 4900-4908). Examples of groups which can react in the absence of a metal catalyst include electron-deficient and strained alkynes, such as dibenzocyclooctyne (DBCO) or bicyclo[6.1.0]nonyne (BCN).

References to “a divalent group derived from a compound selected from . . . hydrazones” are meant to characterize divalent groups —CH═N—NH— resulting from condensation of a carbonyl group with a hydrazine group. In the context of group Y, the divalent group may be formed by reacting a carbonyl group attached to Ab with a hydrazine group attached to T or vice versa.

References to “a divalent group derived from a compound selected from . . . carbonyl-containing compounds” are meant to characterize divalent groups —C(═O)—X—with X representing O, S or NH, resulting from reacting (an activated) carbonyl with a nucleophilic group such as formation of an amide group, ester group, thioester group. In the context of group Y, the divalent group may be formed by reacting a carbonyl-containing group (e.g. —C(═O)—Cl) attached to Ab with a nucleophilic group (e.g. —NH₂) attached to T, or vice versa. Unless specified otherwise, there is no particular limitation to the orientation of the aforementioned divalent group, i.e., to which partner the carbonyl-C binds, and to which partner the X binds.

In the above divalent groups, the term “derivative thereof” means that any hydrogen atom may be replaced by a substituent, as defined hereinbelow, as long as the substitution does not interfere with divalent group formation.

In an analogous manner, the term “derivative” is used to characterize moieties bonded to adjacent moieties, which moieties differ from the molecules from which they are derived only by the structural elements responsible for bonding to adjacent moieties. This may include covalent bonds formed by existing functional groups or covalent bonds and adjacent functional groups newly introduced for this purpose.

Likewise, unless specified otherwise or unless the context dictates otherwise, the expression “derived from” (such as in “derived from a compound”), when used in connection with other groups or moieties, is meant to describe a group or moiety, which is identical to the referenced compound or the like except for the structural modifications necessary for bonding the group or moiety to the one or more adjacent groups or moieties, typically by replacing a hydrogen atom or atomic group by a covalent bond (e.g., replacement of OH in a carboxyl group by a covalent bond upon amide bond formation with an amino group; further examples are given in the below table at the end of the section “Divalent group (X)”).

The term “carbonyl-containing group” as used herein refers to a divalent moiety containing a carbonyl (C═O) or thiocarbonyl (C═S) group. In a preferred embodiment, the said carbonyl-containing group refers to a group represented by one of the following formulae:

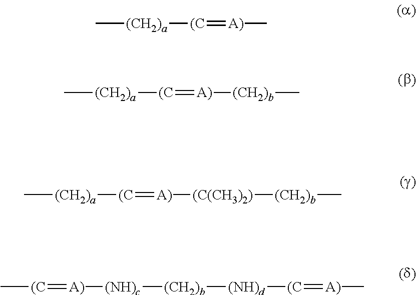

wherein a, b are each independently selected from 0 to 5, preferably 0, 1 or 2, more preferably 0 or 1,
wherein c, d are each selected from 0 or 1, wherein, in one embodiment c is 0 and d is 1, in another specific embodiment c is 1 and d is 0, in yet another specific embodiment both c and d are 1 while in yet another specific embodiment both c and d are 0, and
wherein each A is independently selected from O and S, preferably O.

In a more preferred embodiment, the said carbonyl-containing group refers to a group represented by the formula (α), wherein a is 0, 1 or 2 and A is O. Most preferably, the carbonyl-containing group is —(C═O)—.

In some aspect, the term “amino-containing group” as used herein refers to a divalent moiety containing an amino group, e.g., —N(R)—, wherein R represents a hydrogen atom, an alkyl group or a cycloalkyl group. Preferably, the amino-containing group is a moiety of formula —(CH₂)ₐ—N(R)—, wherein R is a hydrogen atom, an alkyl group or a cycloalkyl group, and a is 0 to 6, preferably 0 or 1, more preferably 0.

The term “solubilizing group” as used herein refers to a hydrophilic group or moiety, which can enhance (improve) the water solubility of the moiety or compound to which it is attached. The solubilizing group can be, for example, a polyalkylene oxide group, such as a polyethylene oxide (PEO) or a polypropylene oxide (PPO) group preferably having from 6 to 200, more preferably 10 to 150 or 12 to 80 repeating units, such as 16 or 40 repeating units, a saccharide group or a moiety comprising one or more ionic or ionizable groups, i.e., functional groups which are charged (anionic or cationic) at physiological pH (7.4), such as moieties derived from amino acids, e.g., from Lys, Glu, Asp, His, Arg, diaminopropionic acid (Dap), diaminobutyric acid (Dab), 2-aminoadipic acid (Aad), carnitine, Orn. Examples of ionic or ionizable groups include ammonium groups, guanidinium groups, sulfate groups, phosphate groups, phosphonate groups, and sulfonate groups. Examples of saccharide groups include monosaccharides, disaccharides and linear or branched oligosaccharides, in particular linear or branched oligosaccharides having 3 to 10 monosaccharide units being linked by glycosidic bond, wherein each of the monosaccharide units in the monosaccharide, disaccharide and oligosaccharide is independently selected from glucose, fructose, mannose, ribose, and galactose.

In the present disclosure, the expression “moiety comprising one or more solubilizing groups” preferably refers to a moiety derived from an amino acid comprising one, two, three or four, preferably one or two, ionic or ionizable groups elected from, e.g., ammonium groups, guanidinium groups, sulfate groups, phosphate groups, phosphonate groups, and sulfonate groups. Such moiety is preferably selected from Lys, Glu, Asp, His, Arg, Dap, Dab, Aad and Orn, more preferably from Arg and His. In further embodiments, the said moiety can consist of an amino acid.

The term “polyalkylene oxide” (or “polyalkylene glycol”, “polyoxyalkylene”) as used herein refers to substances of the general structure HO—(X—O)ₙ—H, wherein X represents an alkylene group having 2 or 3 carbons atoms, and n indicates the number of repeating units, e.g., 6 to 200, 10 to 150, or 12 to 80 repeating units, such as 16 or 40 repeating units, e.g., 17, 18, 20 or 24 PEO repeating units. Thus, the term “polyalkylene oxide group” is to be understood as a divalent group of formula *—O—(X—O)ₙ—**, wherein X and n are as defined above, and * and ** indicate covalent attachment to adjacent moieties. In some embodiments, the term “polyalkylene oxide” can refer to polyethylene oxide (or polyethylene glycol, C₂-polyalkylene oxide), or polypropylene oxide (or polypropylene glycol, C₃-polyalkylene oxide). It is also possible to provide a polyalkylene oxide group, in which two or more different alkylene groups, as defined above, are arranged in a random or block-wise manner.

Unless specified otherwise or the context dictates otherwise, references to groups being “substituted” or “optionally substituted” are to be understood as references to the presence (or optional presence, as the case may be) of at least one substituent selected from F, Cl, Br, I, CN, NO₂, NH₂, NH—C₁₋₆-alkyl, N(C₁₋₆-alkyl)₂, —X—C₁₋₆-alkyl, —X—C₂₋₆-alkenyl, —X—C₂₋₆-alkynyl, —X—C₆₋₁₄-aryl, —X-(5-14-membered heteroalkyl with 1-3 heteroatoms selected from N, O, S), wherein X represents a single bond, —(CH₂)—, —O—, —S—, —S(O)—, —S(O)₂—, —NH—, —CO—, or any combination thereof including, for instance, —C(O)—NH—, —NH—C(O)—. The number of substituents is not particularly limited and may range from 1 to the maximum number of valences that can be saturated with substituents. It is typically 1, 2 or 3 and usually 1 or 2, most typically 1.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise, chiral compounds and moieties may be present in the form of a pure stereoisomer or in the form of a mixture of stereoisomers, including the 50:50 racemate. In the context of the present disclosure, references to specific stereoisomers are to be understood as references to compounds or moieties, wherein the designated stereoisomer is present in at least 90% enantiomeric excess (ee), more preferably at least 95% ee and most preferably 100% ee, wherein % ee is defined as (|R−S|)/(R+S)*100% with R and S representing the amount of moles of the respective enantiomers.

Unless the context dictates otherwise, and/or alternative meanings are explicitly provided herein, all terms are intended to have meanings generally accepted in the art, as reflected by IUPAC Gold Book (status of 1 Nov. 2022), or the Dictionary of Chemistry, Oxford, 8^{th} Ed.

The term “antitumor compound,” as used herein, refers to a compound having an antitumor effect and a substituent group or a partial structure allowing connection to a linker structure. When a part or whole linker is cleaved in tumor cells, the antitumor compound moiety is released to exhibit the antitumor effect of the antitumor compound. As the linker is cleaved at a connecting position to the drug, the antitumor compound can be released in an unmodified structure to exhibit its intrinsic antitumor effect.

The term “native drug” refers to a compound, for which therapeutic efficacy has been established by in vitro and/or in vivo tests. In a preferred embodiment, the native drug is a compound for which therapeutic efficacy has been established by clinical trials. Most preferably, the native drug is a drug that is already commercially available. The type of therapeutic efficacy to be established and suitable tests to be applied depend of course on the type of medical indication to be treated.

The antitumor compound can be released in an unmodified (native) form, or in a modified form insofar as a group or moiety remains attached thereto after cleavage by Cathepsin B, e.g., released as a moiety D-Dxx-Dyy. It is advantageous if the remaining modified drug D-Dxx-Dyy is pharmacologically active. Pharmacological activity in this connection means that the released modified drug, e.g., the moiety D-Dxx-Dyy, retains at least 20%, preferably at least 50%, more preferably at least 80% of the pharmacological activity of the native drug when released intracellularly by the conjugate. To ensure realistic conditions, such a test for activity should not be made via cell cytotoxicity comparison of the released modified drug and the native drug because these conditions require entry of the modified drug into the cell, which may introduce a cell permeability bias. Differences in permeability between these two entities are not relevant here due to the intracellular release of the modified drug. It may be possible to compare activities of the modified drug and the native drug in a cell-free binding assay to determine Ki values (binding affinities) for the appropriate target receptor of the drug. If it is not possible to determine the Ki values, one may compare the IC50 of the cytotoxicity in HER2+ cell lines of two ADCs with the exact same linker system, one designed to release the modified drug and one to release the native drug.

When referring to specific classes of drug molecules, such as an antineoplastic agent, a topoisomerase inhibitor, an RNA-polymerase II inhibitor, a DNA cleaving agent, an antimitotic agent or microtubule disruptor, an anti-metabolite, a kinase inhibitor, an immunomodulatory agent, or an anti-infectious disease agent, these terms are intended to have the meaning generally accepted in the field of medicine, as reflected, for instance, in the Mosby's Medical Dictionary, Mosby, Elsevier 10^{th} ed. (2016), or in Oxford Textbook of Oncology, David J. Kerr, OUP Oxford 3^{rd} ed. (2016).

Accordingly, the drug to be used in the ligand-drug-conjugate of the present disclosure can be a native drug (e.g., a drug naturally containing one or more functional groups allowing covalent attachment to the conjugate), or can be a drug chemically modified to incorporate one functional group (e.g., a group selected from hydroxyl, carboxyl, amino and thiol groups) allowing covalent attachment(s) to an adjacent group or moiety) provided that the modified drug is pharmacologically active. Pharmacological activity in this connection means at least 20%, preferably at least 50%, more preferably at least 80% of the pharmacological activity of the native drug.

The term “auristatin” (or “auristatin analog”) refers to a class of compounds structurally related to the naturally occurring pentapeptide dolastin 10. The auristatin analogs (auristatins) as used herein satisfy the following formula:

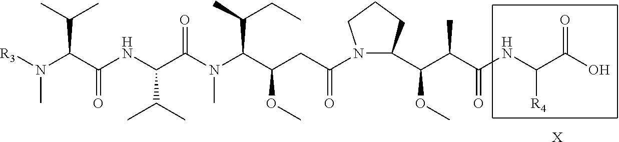

wherein R₃ represents a hydrogen atom or an alkyl group having 1 to 10 carbon atoms, preferably a hydrogen atom or a methyl group; and R₄ represents the side chain of any natural or unnatural amino acid.

In specific embodiments, the compound of the present disclosure makes use of certain auristatin analogs. Typical examples of such auristatin analogs include monomethyl auristatin E (MMAE) and monomethyl auristatin F (MMAF). In the following, the expression “auristatin analog”, when characterizing analogs to be used in accordance with the present disclosure, refers, in particular, to auristatin X, wherein the C-terminal amino acid X (as shown above) is selected from Phe (in which case, the auristatin analog is auristatin Phe/F (AF)), Cit (in which case, the auristatin analog is auristatin Cit (ACit)), Arg (in which case, the auristatin analog is auristatin Arg (AArg)), Lys (in which case, the auristatin analog is auristatin Lys (ALys)), Orn (in which case, the auristatin analog is auristatin Orn (AOrn)), Dab (in which case, the auristatin analog is auristatin Dab (ADab)) and Dap (in which case, the auristatin analog is auristatin Dap (ADap)). The auristatin analogs as used herein (e.g., AF, ACit, AArg, ALys, AOrn, ADab, ADap) are to be considered as native drugs, in addition to the native drugs as defined above.

As used herein, the term “pharmaceutical composition” refers to a composition including the antibody-drug conjugate of the present disclosure and optionally together with a pharmaceutically acceptable carrier. The pharmaceutically acceptable carrier for use in the pharmaceutical composition of the present disclosure are well known to those of ordinary skill in the art and are selected based upon a number of factors such as the particular antitumor compound used, and its concentration, stability and intended bioavailability, the disease, disorder or condition being treated with the composition, the subject, its age, size and general condition, and the route of administration. For instance, the antibody-drug conjugate of the present disclosure may be mixed with a solvent such as a sterilized liquid (including water and oil (oil derived from petroleum, animals, vegetables, or synthetic oil (e.g., peanut oil, soybean oil, mineral oil, sesame oil, etc.)), a saline, a dextrose aqueous solution, or a glycerol aqueous solution, and additives such as a moisturizer, an emulsifier, or a pH buffer, and the like, so as to prepare the pharmaceutical composition of the present disclosure.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer may refer to inhibiting growth and/or spread of the cancer cells, killing the cancer cells, or shrinking the tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±20% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. Conversely, it is understood that reference to a quantitative value may also refer to “an integer” of that value if this is technically meaningful. For example, where in Formula (1), reference is made to an indicia “m” having a particular value (e.g., 1 to 5), m is to be understood as an integer of the specified value (e.g., an integer of 1 to 5). Likewise, where reference is made to indices p1, p2, p3, p4, o′, etc., these indices may be understood as an integer of the specified value (as long as this is technically meaningful). However, while indicia like “m” may be understood in a technically meaningful manner as an integer when looking at individual molecules and/or a composition containing only a single type of molecules, it is not excluded in the context of the present disclosure to provide such molecules in the form of a mixture comprising two or more types of molecules of the disclosure, which may differ from each other with respect to such indicia like “m”. The resulting average value of the indica like “m” is not necessarily an integer. Nevertheless, such mixtures are intended to be encompassed by the present disclosure irrespective whether reference is made to an “integer” or not. If reference is made to an integer, this should be understood as characterizing an individual type of molecule, which may be provided in pure form or be mixed with other molecules of the disclosure.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

The term “effective amount” of “therapeutically effective amount” refers to an amount of a therapeutic agent (e.g., antibody-drug conjugates) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

The terms “subject,” “patient,” “individual,” and the like are used interchangeably herein, and refer to any animal, any mammalian subject, or cells thereof whether in vitro or in situ, amenable to the methods described herein. In certain non-limiting embodiments, the patient, subject or individual is a human.

### BRIEF DESCRIPTION OF DRAWINGS

FIGS. 1a, 1b, 1c and 1d show dose-dependent cytotoxic activity of Ab1-Linker-DM1 (FIG. 1a), Ab1-Linker-AF (FIG. 1b), Ab2-Linker-AF (FIG. 1c) Ab1-Linker-MMAF, and Ab3-Linker-MMAF (FIG. 1d) against BCAM positive human cancer cell lines.

FIG. 2 shows A431 tumor growth curve after treating Ab1-Linker-AF.

FIG. 3 shows relative tumor growth rate on day 14 after treating Ab1-Linker-AF.

FIG. 4a shows an exemplary structure of the antibody-drug conjugate of the present disclosure (Antibody-Drug Conjugate No. 1 (Ab-Linker-AF)). In FIG. 4a, Ab is an anti-BCAM antibody or an antigen binding fragment thereof as described further below, and n is 1 to 10.

FIG. 4b shows an exemplary structure of the antibody-drug conjugate of the present disclosure (Antibody-Drug Conjugate No. 2 (Ab-Linker-MMAF)). In FIG. 4b, Ab is an anti-BCAM antibody or an antigen binding fragment thereof as described further below, and n is 1 to 10.

FIG. 5 shows an exemplary structure of the antibody-drug conjugate of the present disclosure (Antibody-Drug Conjugate No. 3 (Ab-Linker-DM1)). In FIG. 5, Ab is an anti-BCAM antibody or an antigen binding fragment thereof as described further below, and n is 1 to 10.

FIGS. 6a, 6b and 6c show the results of SE-HPLC of Ab (FIG. 6a: the results of Ab1;

FIG. 6b: the results of Ab2; and FIG. 6c: the results of Ab3). Here, the sample was loaded on the size-exclusion column (TSKgel G3000SWXL, 7.8×300 mm (TOSOH)).

FIGS. 7a, 7b, and 7c show the results of SDS-PAGE of Ab (FIG. 7a: the results of Ab1; FIG. 7b: the results of Ab2; and FIG. 7c: the results of Ab3). Here, the samples and their loading amounts in the 3 lanes, from left to right, were: M, marker, 5 μL; 1, non-reducted, 3 μg; 2, reducted, 3 μg.

FIG. 8 shows the SEC chromatogram (aggregation content attribution) of HG4K IgG4-Linker-DM1 conjugate.

FIG. 9 shows the SEC chromatogram (aggregation content attribution) of HG4K IgG4-Linker-AF conjugate.

FIG. 10 shows the SEC chromatogram (aggregation content attribution) of Ab1-Linker-DM1 conjugate.

FIG. 11 shows the SEC chromatogram (aggregation content attribution) of Ab1-Linker-AF conjugate.

FIG. 12a shows the SEC chromatogram (aggregation content attribution) of Ab2-Linker-AF conjugate.

FIG. 12b shows the SEC chromatogram (aggregation content attribution) of Ab1-Linker-MMAF conjugate.

FIG. 12c shows the SEC chromatogram (aggregation content attribution) of Ab3-Linker-MMAF conjugate.

FIG. 12d shows the SEC chromatogram (aggregation content attribution) of HG4 IgG4-Linker-MMAF conjugate.

FIG. 13 shows the MS native deglycosylated (DAR attribution) of HG4K IgG4-Linker-DM1 conjugate.

FIG. 14 shows the MS native deglycosylated (DAR attribution) of HG4K IgG4-Linker-AF conjugate.

FIG. 15 shows the MS native deglycosylated (DAR attribution) of Ab1-Linker-DM1 conjugate.

FIG. 16 shows the MS native deglycosylated (DAR attribution) of Ab1-Linker-AF conjugate.

FIG. 17a shows the MS native deglycosylated (DAR attribution) of Ab2-Linker-AF conjugate.

FIG. 17b shows the MS native deglycosylated (DAR attribution) of Ab1-Linker-MMAF conjugate.

FIG. 17c shows the MS native deglycosylated (DAR attribution) of Ab3-Linker-MMAF conjugate.

FIG. 17d shows the MS native deglycosylated (DAR attribution) of HG4K IgG4-Linker-MMAF conjugate.

FIG. 18 shows the indirect ELISA binding affinity of Ab1 and Ab2 against recombinant human BCAM protein

FIG. 19 shows the sandwich ELISA binding affinity of Ab1 and Ab3 against recombinant human BCAM protein.

FIGS. 20a and 20b show the binding affinity of Ab against BCAM-overexpressing HEK293 cells (FIG. 20a: the results of Ab1 and Ab2; and FIG. 20b: the results of Ab3).

FIG. 21 shows the internalization property of Ab1 into MKN-1 cancer cells.

### MODE FOR THE INVENTION

### Detailed Description

### 1. The Antibody-Drug Conjugate

The present disclosure provides an antibody-drug conjugate of Formula (1) having the structure of Ab-(L-(D)ₘ)ₙ or a pharmaceutically acceptable salt thereof.

### (1) Anti-BCAM Antibody

In Formula (1), Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6.

In one embodiment, in the anti-BCAM antibody, the heavy chain variable region includes a sequence of SEQ ID NO: 7, and the light chain variable region includes a sequence of SEQ ID NO: 8. In one embodiment, in the BCAM antibody, the heavy chain variable region consists of a sequence of SEQ ID NO:7, and the light chain variable region consists of a sequence of SEQ ID NO:8. In one embodiment, in the anti-BCAM antibody, the heavy chain includes or consists of a sequence of SEQ ID NO: 9, and the light chain includes or consists of a sequence of SEQ ID NO: 11. In one embodiment, in the anti-BCAM antibody, the heavy chain includes or consists of a sequence of SEQ ID NO: 10, and the light chain includes or consists of a sequence of SEQ ID NO: 11. In one embodiment, in the anti-BCAM antibody, the heavy chain includes or consists of a sequence of SEQ ID NO: 12, and the light chain includes or consists of a sequence of SEQ ID NO: 11.

In addition, the antigen binding fragment thereof may be an antibody fragment selected from the group consisting of a Fab fragment, a Fab′ fragment, a Fab′-SH, a Fv fragment, a scFv fragment, a F(ab′)₂ fragment, a VL fragment, a VH fragment, a ScFv-Fc fragment, and a (ScFv)₂-Fc fragment, a diabody, a linear antibody, a fragment produced by a Fab expression library, an anti-idiotypic (anti-Id) antibody, a complementary determining region (CDR), and an epitope-binding fragment. In another embodiment, the anti-BCAM antibody is a chimeric antibody, a humanized antibody, or a human antibody. In one embodiment, the antibody or fragment thereof according to the present disclosure is the mature version of any of the antibodies or fragments thereof disclosed therein. In one embodiment, the antibody according to the present disclosure may be human IgG1, IgG2, IgG3, IgG4 type or a mutant thereof. In another embodiment, the antibody of the present disclosure may be human IgG1, IgG4 type or a mutant thereof. The sequence information referred to herein is as follows.

| | |
|---|---|
| SEQ ID NO: 1 | GFTFSDYYMS |
| SEQ ID NO: 2 | YTSSSGSTIYYADSVKG |
| SEQ ID NO: 3 | DITAFDI |
| SEQ ID NO: 4 | TGTSSDVGGYNYVS |
| SEQ ID NO: 5 | EVNKRPS |
| SEQ ID NO: 6 | CSYAGSNNYV |
| SEQ ID NO: 7 | EVQLVQSGGGLVKPGGSLRLSCAASGFTFSDYYMSWIRQAPGKGL |
| | EWVSYTSSSGSTIYYADSVKGRFTISRDNAKNSLYLQMNSLRAED |
| | TAVYYCARDITAFDIWGQGTMVTVSS |
| SEQ ID NO: 8 | LPVLTQPPSASGSPGQSVTISCTGTSSDVGGYNYVSWYQQHPGKA |
| | PKLMIYEVNKRPSGVPDRESGSKSGNTASLTVSGLQAEDEADYYC |
| | CSYAGSNNYVFGTGTKVTVL |
| SEQ ID NO: 9 | EVQLVQSGGGLVKPGGSLRLSCAASGETFSDYYMSWIRQAPGKGL |
| | EWYSYTSSSGSTIYYADSVKGRFTISRDNAKNSLYLQMNSLRAED |
| | TAVYYCARDITAFDIWGQGTMVTVSSASTKGPSVFPLAPCSRSTSE |
| | STAALGCLVKDYFPEPVTVSWNSGALTSGVHTEPAVLQSSGLYSL |
| | SSVVTVPSSSLGTKTYTCNVDHKPSNTKVDKRVESKYGPPCPPCPA |
| | PEFLGGPSVFLFPPKPKDTLMISRTPEVTCVVVDVSQEDPEVQENW |
| | YVDGVEVHNAKTKPREEQFNSTYRVVSVLIVLHQDWLNGKEYK |
| | CKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTV |
| | DKSRWQEGNVFSCSVMHEALHNHYTQKSLSLSLGK |
| SEQ ID NO: 10 | EVQLVQSGGGLVKPGGSLRLSCAASGETFSDYYMSWIRQAPGKGL |
| | EWVSYTSSSGSTIYYADSVKGRFTISRDNAKNSLYLQMNSLRAED |
| | TAVYYCARDITAFDIWGQGTMVTVSSASTKGPSVFPLAPSSKSTSG |
| | GTAALGCLVNDYFPEPVTVSWNSGALTSGVHTFPAVLQSSGLYSL |
| | SSVVTVPSSSLGTQTYICNVNHKPSNTKVDKKVEPKSCDKTHTCP |
| | PCPAPELLGGPSVFLFPPKPKDTLMISRTPEVTGVVVDYSHEDPEVK |
| | FNWYVDGVEVHINAKTKPREEQYNSTYRVVSVLIVLHQDWLNGK |
| | EYKCKVSNKALPAPIEKTISKAKGQPREPQVYTLPPSREEMTKNQV |
| | SLTCLVKGFYPSDIAVEWESNGQPENNYKTIPPVLDSDGSFFLYSK |
| | LTVDKSRWQQGNVFSCSVMHEALHNHYTQKSLSLSPGK |
| SEQ ID NO: 11 | LPVLTQPPSASGSPGQSVTISCTGTSSDVGGYNYVSWYQQHPGKA |
| | PKLMIYEVNKRPSGVPDRESGSKSGNTASLTVSGLQAEDEADYYC |
| | CSYAGSNNYVFGTGTKVTVLGQPKAAPSVTLFPPSSEELQANKAT |
| | LVCLISDFYPGAVTVAWKADSSPVKAGVETTTPSKQSNNKYAASS |
| | YLSLTPEQWKSHRSYSCQVTHEGSTVEKTVAPTECS |
| SEQ ID NO: 12 | EVQLVQSGGGLVKPGGSLRLSCAASGETFSDYYMSWIRQAPGKGL |
| | EWVSYTSSSGSTIYYADSVKGRFTISRDNAKNSLYLQMNSLRAED |
| | TAVYYCARDITAFDIWGQGTMVTVSSASTKGPSVFPLAPSSKSTSG |
| | GTAALGCLVNDYFPEPVTVSWNSGALTSGVHTFPAVLqSSGLYSL |
| | SSVVTVPSSSLGTQIYICNVNHKPSNTKVDKKVEPKSCDKTHTCP |
| | PCPAPLAAGGPSVFLIPPKPKDTLMISRTPEVTCVVVDVSIIEDPEVK |
| | FNWYVDGVEVHNAKTKPREEQYNSTYRVVSVLTVLHQDWLNGK |
| | SYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPGK |

The anti-BCAM antibody and antigen binding fragments thereof may bind to both BCAM protein and BCAM-positive cancer cells with high affinity and may be efficiently internalized into the BCAM expressing cancer cells after binding.

The anti-BCAM antibody and antigen binding fragments thereof may be produced by any methods known in the art such as recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

In one embodiment, the anti-BCAM antibody is Ab1, Ab2 or Ab3 which was prepared as described in Example 1 of the present disclosure. Ab1, Ab2 or Ab3 has the heavy chain variable region including a sequence of SEQ ID NO: 7, and the light chain variable region including a sequence of SEQ ID NO: 8. Ab1 has the heavy chain including or consisting of a sequence of SEQ ID NO: 9, and the light chain including or consisting of a sequence of SEQ ID NO: 11. Ab2 has the heavy chain including or consisting of a sequence of SEQ ID NO: 10, and the light chain including or consisting of a sequence of SEQ ID NO: 11. Ab3 has the heavy chain including or consisting of a sequence of SEQ ID NO: 12, and the light chain including or consisting of a sequence of SEQ ID NO: 11.

### (2) Linker

In Formula (1), L is a linker, and the antitumor compound (D) is conjugated to the anti-BCAM antibody via the linker.

### (2.1) Linker of Formula (2)

In one embodiment of the present disclosure, the linker (L) is represented by Formula (2):

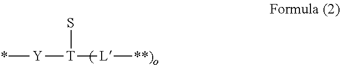

wherein Y is a divalent group comprising one or more atoms selected from C, N, O, P and S, preferably a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein T is a (1+o)- or (2+o)-valent connecting group,
wherein S is an atom or group that is optionally present to saturate a free valency of T,
wherein L′ is a linker capable of being cleaved by Cathepsin B,
wherein o is an integer of 1 to 5, preferably 1 or 2, more preferably 1,
wherein * indicates covalent attachment to the anti-BCAM antibody (Ab), and
wherein ** indicates covalent attachment to one or more antitumor compounds (D).

In a preferred embodiment, the linker (L) is covalently attached to the anti-BCAM antibody or fragment thereof via the side chain of a cysteine comprised in the said antibody or antibody fragment.

(2.1.1) Linker Capable of being Cleaved by Cathepsin B (L′)

The linker of Formula (2) contains a cleavable element (L′), which serves as substrate for specific recognition and cleavage by Cathepsin B, and especially fast cleavage and/or cleavage by the exopeptidase activity of Cat B. The cleavable linker (L′) is covalently attached to the connecting group (T), as well as to one or more antitumor compounds (D).

In one embodiment, the linker capable of being cleaved by Cathepsin B (L′) is represented by Formula (3), or Formula (4):

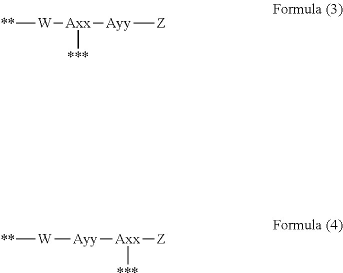

wherein Axx is a trifunctional amino acid, with the proviso that Axx in Formula (3) is not an amino acid in the (D) configuration,
wherein Ayy in Formulae (3) and (4) is an amino acid selected from Phe, Ala, Trp, Tyr, Phenylglycine (Phg), Met, Val, His, Lys, Arg, Citrulline (Cit), 2-amino-butyric acid (Abu), Ornithine (Orn), Ser, Thr, Leu and Ile, or Ayy in Formula (3) is an amino acid selected from homo-tyrosine (homo-Tyr), homo-phenylalanine (homo-Phe), betaphenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, with the proviso that Ayy in Formula (4) is not an amino acid in the (D) configuration,
wherein Z is a group covalently attached to the C-terminus of Ayy or Axx selected from —OH and —N(H)(R), wherein R represents a hydrogen atom, an alkyl group or a cycloalkyl group,
wherein W is a drug-carrying unit,
wherein ** indicates covalent attachment to one or more moieties D, and
wherein *** indicates covalent attachment to T,
if more than one linker (L′) is present, each linker is independently selected from the aforementioned linkers of Formula (3) and Formula (4).

Without wishing to be bound by theory, it is believed that Ayy provides the compound of the present disclosure with the structural features for specific recognition and cleavage by the exopeptidase activity of Cat B. As a result, the compound can release the drug at a significantly higher rate as compared to a compound cleaved by the endopeptidase activity of Cat B, e.g., a compound comprising a Val-Cit-PABC linker system.

Furthermore, the presence of a sterically demanding moiety (Ab-Y-T(S) construct) on the side chain of residue Axx in Formula (3)/(4) has no detrimental effect on the binding affinity of the compound of the present disclosure to Cat B, nor on the cleavage rate of the compound by the exopeptidase mechanism of Cat B. Without wishing to be bound to any theory, it is believed that the sterically demanding moiety is directed towards the outside of the Cat B binding groove (hydrophobic pocket), thus leading to superior cleavage rate via the exopeptidase mechanism.

In Formulae (3) and (4), it is preferred that at least one, or both of Axx and Ayy is/are defined as follows:
(a) Axx in Formula (3) or (4) is an amino acid selected from Glu, 2-amino-pimelic acid (Apa), 2-amino adipic acid (Aaa), 2,3-diamino-propionic acid (Dap), 2,4-diamino-butyric acid (Dab), Lys, Orn, Ser, amino-malonic acid (Ama), and homo-lysine (homo-Lys), preferably an amino acid selected from Dap, Dab, Lys, Orn and homo-Lys,
(b) Ayy in Formula (3) is an amino acid selected from Phe, homo-Phe, Ala, Trp, Phg, Leu, Val, Tyr, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24, preferably an amino acid selected from Phe, homo-Phe, Tyr, homo-Tyr, Tyr(OR₁) and homo-Tyr(OR₁), more preferably an amino acid selected from Phe and Tyr, or
(c) Ayy in Formula (4) is an amino acid selected from Phe, homo-Phe, Ala, Trp, Phg, Leu, Val, Tyr and Ser, preferably an amino acid selected from Phe, home-Phe and Ser, more preferably an amino acid selected from Phe and Ser.

In Formulae (3) and (4), it is more preferred that at least one, or both of Axx and Ayy is/are defined as follows:
(a) Axx in formula (3) or (4) is an amino acid selected from Dap, Dab, Lys, Orn and homo-Lys,
(b) Ayy in Formula (3) is an amino acid selected from Phe and Tyr, or
(c) Ayy in Formula (4) is an amino acid selected from Phe and Ser.

### (2.1.2) Drug-Carrying Unit (W)

The drug-carrying unit (W) is a moiety that connects a C-terminal dipeptide comprised in the cleavable linker (L′), e.g., moiety Axx-Ayy in Formula (3) or moiety Ayy-Axx in Formula (4), to one or more antitumor compounds (D).

In a preferred embodiment, the drug-carrying unit (W) is a group represented by Formula (5):

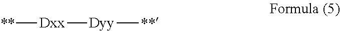

wherein Dxx is absent or an amino acid having a hydrophobic side chain,
wherein Dyy is absent, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein ** indicates covalent attachment to D, and
wherein **′ indicates covalent attachment to the N-terminus of Axx or Ayy.

In Formula (5), it is preferred that at least one, e.g., one or two, of Dxx and Dyy is/are defined as follows:
(a) Dxx is an amino acid selected from Phe, Val, Tyr, homo-Phe and Ala,
(b) Dyy is absent, or an amino acid selected from Arg, Lys, Cit, Orn, Dap and Dab.

In Formula (5), it is more preferred that at least one, e.g., one or two, of Dxx and Dyy is/are defined as follows:
(a) Dxx is an amino acid selected from Phe and Val,
(b) Dyy is absent, or an amino acid selected from Arg and Cit.

In some embodiments, Dxx may contain a further element such that the single covalent bond or amino acid having a hydrophobic side chain is optionally attached to D via a divalent moiety selected from maleimides, triazoles, hydrazones, carbonyl-containing groups, and derivatives thereof, preferably via a divalent maleimide derivative such as an opened hydrolyzed maleimide derivative.

In another preferred embodiment, the drug-carrying unit (W) is a group represented by Formula (6), or Formula (7):

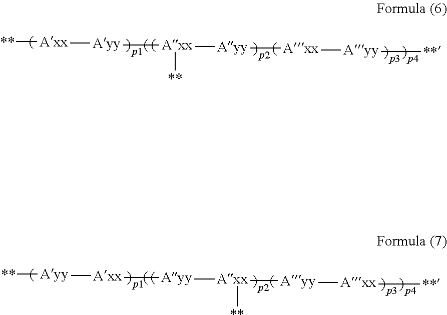

wherein A″xx is a trifunctional amino acid, with the proviso that A″xx in Formula (6) is not an amino acid in the (D) configuration,
wherein A′yy is an amino acid selected from Phe, Ala, Trp, Tyr, Phg, Met, Val, His, Lys, Arg, Cit, Abu and Orn, with the proviso that A′yy in Formula (7) is not an amino acid in the (D) configuration; if more than one A′yy are present, each A′yy is independently selected from the aforementioned amino acids,
wherein A″yy is an amino acid selected from Phe, Ala, Trp, Tyr, Phg, Met, Val, His, Lys, Arg, Cit, Abu and Orn, with the proviso that A″yy in Formula (7) is not an amino acid in the (D) configuration; if more than one A″yy are present, each A″yy is independently selected from the aforementioned amino acids,
wherein A′″yy is an amino acid selected from Phe, Ala, Trp, Tyr, Phg, Met, Val, His, Lys, Arg, Cit, Abu and Orn, with the proviso that A′″yy in Formula (7) is not an amino acid in the (D) configuration; if more than one A′″yy are present, each A′″yy is independently selected from the aforementioned amino acids,
wherein A′xx is an amino acid, with the proviso that A′xx in Formula (6) is not an amino acid in the (D) configuration,
wherein A′″xx is an amino acid, with the proviso that A′″xx in Formula (6) is not an amino acid in the (D) configuration,
wherein p1 is an integer of 0 to 3, preferably 0,
wherein p2 is 0 or 1, preferably 1,
wherein p3 is an integer of 0 to 3, with the proviso that if p2 is 0, p3 is not 0; p3 being preferably 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.

In Formulae (6) and (7), it is preferred that at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Lys, homo-Lys, Cit, Orn, Dap and Dab,
(c) A′″xx is an amino acid selected from Arg, Lys, homo-Lys, Cit, Orn, Dap and Dab,
(d) A′yy is an amino acid selected from Phe, Ala, Trp, Phg and Tyr, preferably an amino acid selected from Phe and Tyr,
(e) A″yy is an amino acid selected from Phe, Ala, Trp, Phg and Tyr, preferably an amino acid selected from Phe and Tyr,
(f) A′″yy is an amino acid selected from Phe, Ala, Trp, Phg and Tyr, preferably an amino acid selected from Phe and Tyr.

The peptide of Formula (3)/(6), Formula (3)/(7), Formula (4)/(6) or Formula (4)/(7) acts as a specific substrate for the exopeptidase activity of Cat B. That is, the linker of Formula (3) or Formula (4) described herein can be cleaved at its N-terminus by Cat B, releasing the drug-carrying unit (W). When W is a moiety of Formula (6) or Formula (7), it can in turn be cleaved by Cat B, thus releasing the antitumor compound (D) attached to the N-terminus of A′xx or A′yy, and one or more moieties containing (A″xx(D)-A″yy)/(A″yy-A″xx(D)). In some aspects of the present disclosure, the moiety containing (A″xx(D)-A″yy)/(A″yy-A″xx(D)) exhibits pharmacological (antitumor) activity.

In some aspects of the present disclosure, each moiety containing (A″xx(D)-A″yy)/(A″yy-A″xx(D)) can be “self-immolative” insofar as it can undergo intramolecular aminolysis (i.e., five- or six-membered ring formation, or diketopiperazine (DKP) formation), releasing the moiety (D) as a product. When p2>1, the peptide (A″xx(D)-A″yy)ₚ₂/(A″yy-A″xx(D)ₚ₂ acts as a substrate for Cat B, which can cleave the (p2-1) amide bonds between amino acids A″yy-A″xx/A″yy-A″xx, thus releasing p2 dipeptides (A′xx(D)-A″yy)/(A″yy-A″xx(D)). In some aspects, each dipeptide can in turn undergo intramolecular aminolysis (A″xx(D)-A″yy) or DKP formation (A″yy-A″xx(D)), releasing p2 moieties (D) as product.

Accordingly, when the drug-carrying unit (W) represents a peptide of Formula (6) or Formula (7), the linker can release two or more drug molecules (of the same or different drugs) and thus permits accomplishing a high DAR such that the overall pharmacological activity can be enhanced. The drug release can occur according to a multi-step mechanism. For instance, (W) can be first released from the compound of Formula (I), and then act as a substrate for Cat B releasing moiety (D) and, eventually, p2 moieties containing peptide (A″xx(D)-A″yy)/(A″yy-A″xx(D)), which can be pharmacologically active as such (e.g., intra-antitumor compounds) and/or undergo intramolecular aminolysis, DKP formation or hydrolysis to release p2 moieties (D).

The compound of the present dislcosure is typically stable in an extracellular environment (e.g. in plasma) in the absence of Cat B (i.e., the enzyme capable of cleaving the linker). However, upon exposure to Cat B, the linker (L′) is recognized and cleaved initiating, eventually, the spontaneous self-immolative aminolysis resulting in the cleavage of the bond covalently linking the self-immolative moiety, e.g., A″xx-A″yy, to the drug, to thereby achieve release of the compound (D) in its pharmacologically active form. Self-immolative aminolysis can occur if A″xx represents an amino acid such as Glu, Aaa, Dap, Dab, Ser, Thr, homoSer, homo-Thr.

In yet another preferred embodiment, the drug-carrying unit (W) is a group represented by Formula (8):

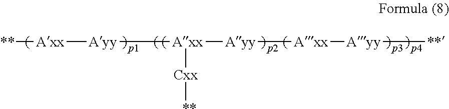

wherein A″xx is a trifunctional amino acid selected from Glu, α-amino adipic acid (Aaa), Dap, Ser, Thr, homo-serine (homo-Ser), homo-threonine (homo-Thr) and amino malonic acid (Ama), with the proviso that A″xx is not an amino acid in the (D) configuration,
wherein Cxx is a single covalent bond unless A″xx is Ama; if A″xx is Ama, Cxx is Pro or an N-methyl amino acid, the N-terminus of Cxx binds to a carboxyl end of Ama and the C-terminus of Cxx is covalently attached to one moiety D,
wherein A′yy, A″yy and A′″yy are each independently an amino acid selected from Phe, Ala, Trp, Tyr, Phg, Met, Val, His, Lys, Arg, Cit, Abu, Orn,
wherein A′xx and A′″xx are each independently an amino acid, with the proviso that A′xx and A′″xx are not an amino acid in the (D) configuration,
wherein p1 is 0 or 1, preferably 0,
wherein p2 is 0 or 1, preferably 1,
wherein p3 is an integer of 0 to 3, with the proviso that if p2 is 0, p3 is not 0; p3 being preferably 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.

In Formula (8), it is preferred that at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Dap, Dab, Lys, Orn and homo-Lys,
(c) A′″xx is an amino acid selected from Arg, Lys, homo-Lys, Cit, Orn, Dap and Dab,
(d) A′yy is an amino acid selected from Phe, Ala, Trp, Phg and Tyr, preferably an amino acid selected from Phe and Tyr,
(e) A″yy is an amino acid selected from Phe, Ala, Trp, Phg and Tyr, preferably an amino acid selected from Phe and Tyr,
(f) A′″yy is an amino acid selected from Phe, Ala, Trp, Phg and Tyr, preferably an amino acid selected from Phe and Tyr.

It is well established that peptides and proteins that possess a Pro residue at the penultimate N-terminal position undergo non-enzymatic aminolysis, resulting in DKP-formation. The mechanism of DKP formation involves nucleophilic attack of the N-terminal nitrogen on the carbonyl of the second amino acid. This intramolecular aminolysis proceeds readily and plays an important role in the biosynthetic pathway of biologically active cyclic dipeptides such as c(His-Pro), which are found throughout the central nervous system, peripheral tissues, and body fluids. In the dipeptide (Ama(Cxx-D)-A″yy), the mechanism of DKP formation involves nucleophilic attack of the N-terminal nitrogen on the side chain of Cxx, thus releasing moiety D.

The peptide of formula (3)/(8) or Formula (4)/(8) acts as a substrate for the exopeptidase activity of Cat B, releasing a peptide-containing moiety having Formula (8), which in turn can be cleaved by Cat B to release moiety (D) and a moiety containing (A″xx(Cxx-D)-A″yy). The peptide (A′xx(Cxx-D₂)-A′yy) is a “self-immolative” moiety, which can undergo intramolecular aminolysis (i.e., five- or six-membered ring formation, or diketopiperazine (DKP) formation), releasing moiety (D) as a product. When p2>1, the peptide (A″xx(Cxx-D)-A″yy)ₚ₂ acts as a substrate for Cat B, which can cleave the (p2-1) amide bonds between amino acids A″yy and A″xx, thus releasing p2 peptides (A″xx(Cxx-D)-A″yy). Each peptide (A″xx(Cxx-D)-A″yy) can, in turn, undergo intramolecular aminolysis, releasing p2 moieties (D) as product.

Accordingly, when the drug-carrying unit (W) is a peptide of Formula (8), drug release occurs according to a multi-step mechanism, for instance (W) can be first released from the compound of Formula (I) and then act as a substrate for Cat B releasing moiety (D) and p2 peptides (A″xx(Cxx-D)-A″yy), which finally undergo intramolecular aminolysis to release p2 moieties (D). In the peptide (Ama(Cxx-D)-A″yy), the mechanism of DKP formation (intramolecular aminolysis) involves nucleophilic attack of the N-terminal nitrogen of Ama on the ester carbonyl of Cxx, thus releasing (D).

### (2.1.3) Connecting Group (T)

The connecting group (T) represents a (1+o)- or (2+o)-valent, e.g., 2-, 3-, 4-, 5-, 6-valent, connecting group. The connecting group connects the antibody or fragment thereof via the divalent group (Y) to the moiety (S) (if present) and one or more (o) cleavable moieties (L′) thereby forming a linear or branched structure. Preferably, the connecting group (T) is a 2-, 3- or 4-valent group. More preferably, the connecting group (T) is a 2- or 3-valent group. T can be linked to Y, S and L′, e.g., via chemoselective ligation procedures for amide bond formation, via “click chemistry” (e.g., azide-alkyne cycloaddition), or the like.

In some embodiments, T acts as a moiety for multiple drug attachment. It can be a small organic group with two or more valencies having, for instance, a molecular weight of 200 Da or less or even only 100 Da or less, but it can also be a more complex and/or larger moiety derived from functional polymers, copolymers, dendrimers, or synthetic constructs including multiple reactive groups for attachment to L′.

In some aspects of the present disclosure, the connecting group (T) is selected such that it is stable to hydrolysis, meaning that typically less than 20% and preferably less than 10% of a test compound undergoes hydrolysis in phosphate-buffered saline (PBS) solution pH 7.4 at 37° C. within 24 hours, as determined by HPLC, wherein said test compound is a compound based on multivalent group T, wherein all valencies of T are saturated by hydrogen atoms.

Ideally, the compound of Formula (I), containing the connecting group (T), when taken as a whole, also shows such stability to hydrolysis, i.e., it is preferred that less than 20% and more preferably less than 10% of the compound of Formula (I) undergoes hydrolysis in phosphate-buffered saline (PBS) solution pH 7.4 at 37° C. within 24 hours, as determined by HPLC.

In one embodiment, the connecting group is a group comprising at least one moiety derived from a trifunctional amino acid, such as Lys. The connecting group can comprise further (optional) linkers and/or amino acids in addition to the trifunctional amino acid mentioned above, provided that the said further amino acids are not trifunctional amino acids or moieties comprising one or more ionic or ionizable groups. Examples of further linkers include polyoxyalkylene oxides, in particular a polyethylene oxide having from 0 to 20, preferably from 0 to 5 or 0 to 3, such as 1 to 3, ethylene oxide subunits. The further amino acids can, for example, be selected from homo-Phe and Phe. The said further linkers and/or amino acids, if present, are preferably attached to the backbone of the trifunctional amino acid, i.e., to the carboxyl group and/or amino group of a trifunctional amino acid such as Lys. If a moiety S is present, it is preferred that the said further linkers do not act as a solubilizing group (i.e., the effect of the said further linkers on solubility is negligible in comparison with the moiety S). In some aspects, the connecting group consists of a moiety derived from a trifunctional amino acid (i.e., does not include any further linkers and/or amino acids).

In one embodiment, the connecting group (T) is represented by Formula (9):

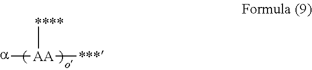

wherein each AA is independently a moiety comprising a trifunctional amino acid,
wherein α indicates covalent attachment of the N-terminus of AA, or the N-terminus of the first AA in case of o′ being 2 to 5, to Y,
wherein o′ is an integer of 1 to 5, with the proviso that o′ is 1 to 4 if another moiety L′ is attached to ***′,
if o′ is 1, the side chain of the trifunctional amino acid is covalently attached to S or L′, the C-terminus being covalently attached to the other moiety L′ or S, respectively,
if o′ is 2, 3, 4 or 5, **** indicates covalent attachment to L′, and ***′ indicates covalent attachment to S.

In Formula (9), it is preferred that each AA is independently a moiety comprising an amino acid selected from N-ε-propargyloxycarbonyl-L-Lysine (Lys(Poc)), Asp, Glu, Orn, Lys, Dab and Dap. More preferably, each AA is independently a moiety comprising an amino acid selected from Lys(Poc), Glu, Orn and Lys. Most preferably, each AA is Lys.

In one embodiment, the connecting group (T) is represented by Formula (10), or Formula (11):

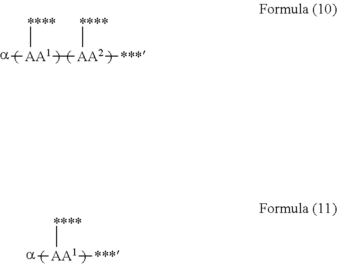

wherein each AA¹ and AA² is independently a moiety comprising a trifunctional amino acid,
wherein α indicates covalent attachment to Y,
wherein, in Formula (11), the side chain of the trifunctional amino acid is covalently attached to L′ or S, the C-terminus is covalently attached to the other moiety S or L′, respectively,
wherein, in Formula (10), **** indicates covalent attachment to L′, and ***′ indicates covalent attachment to S or L′.

In Formulae (10) and (11), it is preferred that each AA¹ and AA² is independently a moiety comprising an amino acid selected from Lys(Poc), Asp, Glu, Orn, Lys, Dab and Dap. More preferably, each AA¹ and AA² is independently a moiety comprising an amino acid selected from Lys(Poc), Glu, Orn and Lys. Most preferably, each AA′ and AA² is Lys.

In one embodiment, the connecting group (T) is represented by Formula (12), or Formula (13):

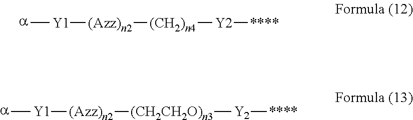

wherein Azz is a moiety comprising one or more solubilizing groups, preferably an amino acid selected from Arg, Dap, Dab, Orn, Lys and carnitine,
wherein Y1 is a single covalent bond, an alkyl group having 1 to 6 carbon atoms, a carbonyl-containing group, or an amino-containing group,
wherein Y2 is a single covalent bond, a carbonyl-containing group such as —(C═O)—, or an amino-containing group such as —N(R)—, wherein R is a hydrogen atom, an alkyl group or a cycloalkyl group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to L′.

In a preferred embodiment, the connecting group (T) is represented by Formula (12′), or Formula (13′):

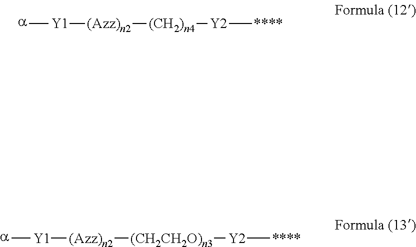

wherein Y1 is a single covalent bond, an alkyl group having 1 to 6 carbon atoms, a carbonyl-containing group, or an amino-containing group, preferably an amino-containing group, more preferably —N(R)— wherein R is a hydrogen atom, an alkyl group or a cycloalkyl group,
wherein Y2 is a carbonyl containing group such as —(C═O)—, or an amino-containing group such as —N(R)— wherein R is a hydrogen atom, an alkyl group or a cycloalkyl group, preferably a carbonyl-containing group, more preferably —(C═O)—,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to L′.

In a preferred embodiment, the connecting group (T) is represented by Formula (10), Formula (11), Formula (13), or Formula (13′). More preferably, the connecting group (T) is represented by Formula (10), Formula (11), or Formula (13′). Most preferably, the connecting group (T) is represented by Formula (11), or Formula (13′).

### (2.1.4) Moiety (S)

S is an atom or group that is optionally present to saturate a free valency of the connecting group (T). For instance, S may be a hydrogen atom, an alkyl group or a cycloalkyl group. In one embodiment, S is a moiety comprising one or more, e.g., 2, 3, 4 or 5, solubilizing groups.

The attachment of a moiety S comprising one or more solubilizing groups may enable to reduce (or prevent) the tendency of the conjugate molecules for aggregation and thus may allow for superior pharmacokinetic properties, e.g., biodistribution, hepatic clearance, even at high DAR (e.g., DAR=8). In some instances, aggregation of the conjugate molecules can be completely suppressed, even at high DAR. Cleavage by the exopeptidase mechanism of Cat B is possible even in the presence of sterically demanding solubilizing groups. Without being bond to any theory, it is believed that the moiety S is directed towards the outside of the Cat B binding groove, thus allowing for superior selectivity and cleavage rate, e.g., via the exopeptidase mechanism. In some aspects, the moiety S is capable to compensate for the potential hydrophobicity of the antitumor compound, such that excellent pharmacokinetic properties can be retained even if multiple antitumor compounds are attached to the linker (e.g., m>1).

In one embodiment, S represents a moiety comprising one or more, e.g., two, three or four, solubilizing groups; wherein each solubilizing group comprised in (S) is independently selected from the group consisting of:
moieties comprising one or more ionic or ionizable groups, such as ammonium, guanidinium, sulfate or sulfonate groups, preferably of moieties derived from Arg, Dap, Dab, Orn, Lys, or carnitine;
saccharide moieties selected from monosaccharides, disaccharides and linear or branched oligosaccharides, in particular linear or branched oligosaccharides having 3 to 10 monosaccharide units being linked by glycosidic bonds, wherein each of the monosaccharide units in the monosaccharide, disaccharide and oligosaccharide is independently selected from glucose, fructose, mannose, ribose, and galactose; and
polyalkylene oxide groups, preferably C₂₋₃ polyalkylene oxide groups, more preferably C₂₋₃ polyalkylene oxide groups independently comprising from 6 to 200, preferably from 10 to 150, more preferably from 12 to 80 repeating units.

There is no particular limitation as to the general arrangement of the solubilizing groups in the moiety (S). Hence, the moiety (S) can have a linear structure, e.g., in which several solubilizing groups are arranged in a random or block-wise manner, a cyclic structure, or a branched structure, e.g., in which several solubilizing groups are attached to a core molecule, such as pentaerythritol or glycerol, in a graft or dendrimeric manner. The moiety (S) can also comprise several blocks, each block independently having a linear or branched structure.

In one aspect, the moiety (S) comprises one or more solubilizing groups arranged in a linear, block-wise manner. For example, the moiety (S) can comprise a structure represented by -(So)ₙ- as illustrated in more detail by the following formula: -(So¹)-(So²)-[ . . . ]-(Soⁿ), wherein each So¹ to Soⁿ represents a solubilizing group, such as a polyalkylene oxide group, e.g., a PEO group having from 6 to 200 repeating units, or a moiety comprising one or more ionic or ionizable groups, such as Arg, and n′ is an integer of 1 to 20, e.g., 1 to 10, with the proviso that directly connected polyalkylene oxide groups of the same structure are to be regarded as multiple repeating units of the same solubilizing group (and not as adjacent So groups). That is, adjacent polyalkylene oxide groups must be of different structure and/or be connected via a functional group like —C(O)—O— or the like in order to be treated as separate So groups.

In another aspect, the moiety (S) comprises one or more solubilizing groups attached to a core molecule, such as pentaerythritol or glycerol, in an untethered, graft or dendrimeric manner. For example, the moiety (S) can have a graft structure represented by —((—Y′—X′(Soₘ))_{n′} —H as illustrated in more detail below:

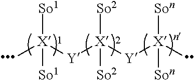

wherein X′ is a (m′+2)-valent, e.g., tri- or tetravalent, group, Y′ is a divalent group, each So is independently selected to be a solubilizing group, such as a polyalkylene oxide group, e.g., a PEO group having from 4 to 600 repeating units, or a moiety comprising one or more ionic groups, m′ is 1, 2, 3, or more and preferably 1 or 2, and n′ is an integer of 1 to 20, e.g., 1 to 10;
or a tree-like, dendrimeric structure represented by —X′(So)_{n′} as illustrated in more detail below:

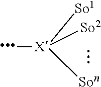

wherein X′ is a n-valent (branching) group, each So¹ to Soⁿ is independently selected to be a solubilizing group as described above, such as a polyalkylene oxide group, e.g., a PEO group having from 4 to 600 repeating units, or a moiety comprising one or more ionic groups, and n′ is an integer of 1 to 20, e.g., 1 to 10.

In one embodiment, the moiety (S) comprises one or more polyethylene oxide groups, wherein preferably each polyethylene oxide group independently comprises from 6 to 200, more preferably from 10 to 150, most preferably from 12 to 80 repeating units.

In a preferred embodiment, the moiety (S) is represented by Formula (23):

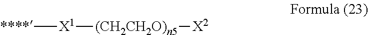

wherein,
n5 is an integer of 6 to 200, preferably 10 to 150, more preferably 12 to 80, such as 24,
**** indicates covalent attachment to (T),
X′ is selected from a single covalent bond, —(C═O)—, and —N(R)— in which R represents a hydrogen atom, an alkyl group or a cycloalkyl group,
X² represents an alkyl group having 1 to 6 carbon atoms, a carbonyl-containing group such an acetyl group or a group of formula —(CH₂)ₙ₆—CO₂H, a thiocarbonyl-containing group, a group of formula —(CH₂)ₙ₆OR, a group of formula —(CH₂)ₙ₆—SO₃H, or an amino-containing group such as a group of formula —(CH₂)ₙ₆—(C=A)-N(R)₂ or —(CH2)ₙ₆—N(R)₂, in which A is O or S, each R is independently selected from a hydrogen atom, an alkyl group and a cycloalkyl group, and n6 is an integer of 1 to 6,
X² being preferably —CH₃, —CH₂CH₂OH, or a group represented by Formula (24):

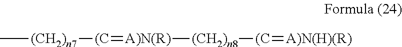

wherein,
each A is independently selected from O and S, preferably O,
each R is independently selected from a hydrogen atom, an alkyl group and a cycloalkyl group, and
n7 and n8 are each independently an integer of 1 to 6, preferably 1 or 2, and
X² being most preferably —CH₃.

If more than one (S) is present, each (S) is preferably a moiety of Formula (23) as described above.

### (2.2) Linker of Formula (14), Formula (15) and Formula (16)

In one embodiment of the present disclosure, the linker (L) comprised in the compound of Formula (1) is represented by Formula (14), or Formula (15):

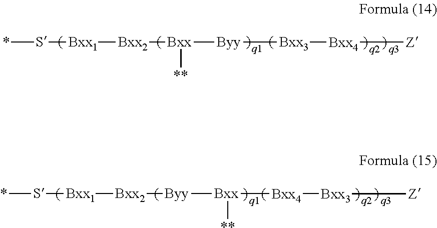

wherein Bxx in Formulae (14) and (15) is a trifunctional amino acid, with the proviso that Bxx in Formula (14) is not in the (D) configuration,
wherein Byy in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂, wherein R₂ is a hydrogen atom or a methyl group and n1 is an integer of 2 to 24, or Byy in Formula (14) is an amino acid selected from homo-Tyr, homo-Tyr(OR₁) wherein R₁ is as defined above, homo-Phe, beta-Phe and beta-homo-Phe; with the proviso that if q1*q3>1 and q2=0, only the C-terminal Byy in Formula (14) may be an amino acid selected from beta-Phe and beta-homo-Phe; with the proviso that Byy in Formula (15) is not in the (D) configuration,
wherein Bxx₁ in Formulae (14) and (15) is a single covalent bond or an amino acid having a hydrophobic or basic side chain,
wherein Bxx₂ in Formulae (14) and (15) is an amino acid having a hydrophobic or basic side chain,
wherein Bxx₃ in Formulae (14) and (15) is an amino acid, with the proviso that Bxx3 in Formula (14) is not in the (D) configuration,
wherein Bxx₄ in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂, wherein R₂ is a hydrogen atom or a methyl group and n1 is an integer of 2 to 24, or Bxx4 in Formula (14) is an amino acid selected from homo-Tyr, homo-Tyr(OR₁), homo-Phe, beta-Phe and beta-homo-Phe; with the proviso that if q2*q3>1, only the C-terminal Bxx4 in Formula (14) may be an amino acid selected from beta-Phe and beta-homo-Phe; with the proviso that Byy in Formula (15) is not in the (D) configuration,
wherein S′ is a divalent group comprising one or more atoms, preferably 1 to 40 atoms, more preferably 1 to 20 atoms, selected from C, N, O, P and S; preferably S′ is a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein Z′ is a group covalently attached to the C-terminus of Byy or Bxx4 in Formula (14) or the C-terminus of Bxx or Bxx3 in Formula (15), which is selected from —OH and —N(H)(R), wherein R represents a hydrogen atom, an alkyl group or a cycloalkyl group,
wherein q1 is an integer of 0 to 5,
wherein q2 is an integer of 0 to 3, with the proviso that if q1 is 0, q2 is not 0,
wherein q3 is an integer of 1 to 5,
wherein q1, q2 and q3 are selected such that m in Formula (1) is an integer of 1 to 5,
wherein * indicates covalent attachment to the anti-BCAM antibody (Ab), and
wherein ** indicates covalent attachment to one moiety D.

In one embodiment of the present disclosure, the linker (L) comprised in the compound of Formula (1) is represented by Formula (16):

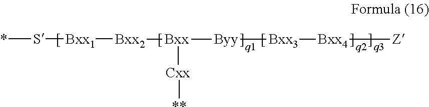

wherein Bxx in Formula (16) is a carboxylic amino acid, or a trifunctional amino acid selected from Dap, Dab, Ser, Thr, Lys, Orn, homo-Lys, homo-Ser and homo-Thr, with the proviso that Bxx is not in the (D) configuration,
wherein Cxx is a single covalent bond unless Bxx is Ama, if Bxx is Ama, Cxx is Pro or an N-methyl amino acid, the N-terminus of Cxx binds to a carboxyl group of Ama and the C-terminus of Cxx is covalently attached to one moiety D,
wherein Byy in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24; with the proviso that if q1*q3>1 and q2=0, only the C-terminal Byy may be an amino acid selected from beta-Phe and beta-homo-Phe,
wherein Bxx₁ in Formula (16) is a single covalent bond or an amino acid having a hydrophobic or basic side chain,
wherein Bxx₂ in Formula (16) is an amino acid having a hydrophobic or basic side chain,
wherein Bxx₃ in Formula (16) is an amino acid, with the proviso that Bxx3 is not in the (D) configuration,
wherein Bxx₄ in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24; with the proviso that if q2*q3>1, only the C-terminal Bxx4 may be an amino acid selected from beta-Phe and beta-homo-Phe,
wherein S′ is a divalent group comprising one or more atoms, preferably 1 to 40 atoms, more preferably 1 to 20 atoms, selected from C, N, O, P and S; preferably S′ is a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein Z′ is a group covalently attached to the C-terminus of Byy or Bxx4 in Formulae (14) and (16) or the C-terminus of Bxx or Bxx3 in Formula (15), which is selected from —OH and —N(H)(R), wherein R represents a hydrogen atom, an alkyl group or a cycloalkyl group,
wherein q1 is an integer of 0 to 5,
wherein q2 is an integer of 0 to 3, with the proviso that if q1 is 0, q2 is not 0,
wherein q3 is an integer of 1 to 5,
wherein q1, q2 and q3 are selected such that m in Formula (1) is an integer of 1 to 5,
wherein * indicates covalent attachment to the anti-BCAM antibody (Ab), and
wherein ** indicates covalent attachment to one moiety D.

In Formulae (14), (15) and (16), it is preferred that at least one, e.g., one, two, three, four, five or six, of Bxx, Byy, Bxx₁, Bxx₂, Bxx₃ and Bxx₄ is/are defined as follows:
(a) Bxx is an amino acid selected from Dap, Dab, Lys, Orn, Ser, Glu, Ama, Thr, Tyr, Aaa, homo-Ser and homo-Thr, preferably an amino acid selected from Lys and Dab, more preferably Lys,
(b) Byy is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24, preferably an amino acid selected from Phe, Tyr and Tyr(OR1), if q1*q3>1, Byy represents preferably Tyr or Tyr(OR₁),
(c) Bxx₁ is a single covalent bond, or an amino acid selected from Phe, homo-Phe, Phg, Val, Ser, Tyr, Ala, Leu and Ile, preferably an amino acid selected from Phe, homo-Phe, Tyr and Val, more preferably an amino acid selected from Phe, homo-Phe and Tyr,
(d) Bxx₂ is an amino acid selected from Arg, Lys, Cit, Val, Leu, Ser, Ala, Gly, His, Gln, Phg and Phe, preferably an amino acid selected from Arg, Lys, Cit and Phe, more preferably an amino acid selected from Arg and Cit,
(e) Bxx₃ is an amino acid selected from Phe, homo-Phe, Phg, Val, Ser, Tyr, Ala, Leu and Ile, preferably an amino acid selected from Phe, homo-Phe, Tyr and Val, more preferably an amino acid selected from Phe, homo-Phe and Tyr,
(f) Bxx₄ is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24, preferably an amino acid selected from Phe, Tyr and Tyr(OR1), if q1*q3>1, Byy represents preferably Tyr or Tyr(OR₁).

### (3) Divalent Group Y

The conjugate of formula (1) contains a divalent group Y comprising one or more atoms selected from C, N, O, P and S. The divalent group connects the BCAM antibody or antigen binding fragment thereof (Ab) to the branching group T. The divalent group is typically attached to the side chain of an amino acid contained in Ab, such as Cys. Preferably, Y is a divalent group derived from a compound selected from maleimides, triazoles, hydrazones, carbonyl-containing compounds, and derivatives thereof. More preferably, Y is a divalent group derived from maleimides and derivatives thereof, such as opened hydrolyzed maleimide derivatives. Most preferably, Y is a divalent group derived from an opened hydrolyzed maleimide.

Hydrolysis of a maleimide attachment is typically performed under basic conditions as a final step of the conjugation of the maleimide derivative to Ab, e.g., under the conditions described above.

In some instances, when n in the conjugate of formula (1) is more than 1, said conjugate may comprise a mixture of (closed) maleimide derivatives (Y) and opened hydrolyzed maleimide derivatives (Y) attached to Ab. Accordingly, in the conjugates described herein in which a group R is attached to the moiety Ab via a maleimide (as shown below, left hand side), hydrolysis may be carried out such that, when n is more than 1, a conjugate of the invention may comprise both closed maleimide attachments (A) and opened hydrolyzed maleimide attachments (B) (as shown below, left hand side) to Ab.

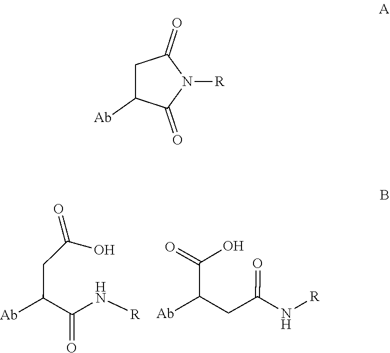

It is preferred that in the conjugate of formula (1) wherein n is more than 1, at least 50% of the Y attachments to Ab are opened hydrolyzed maleimide attachments (B), the remaining attachments being closed maleimide attachments (A). In some instances, at least 70%, at least 75%, at least 80%, at least 90%, at least 95%, preferably at least 98% of the Y attachments to Ab are opened hydrolyzed maleimide attachments (B).

The presence of one or more opened hydrolyzed maleimide attachments (even if present together with one or more closed maleimide attachments) can contribute to the stability and therapeutic efficacy of the conjugates of the invention. Without being bound to any theory, it is believed that the opened hydrolyzed maleimide attachment, e.g., may prevent a retro-Michael reaction that causes the liberation of reactive maleimide in the circulation and ultimately leads to transfer of linker-payload to other thiol-containing molecule in the body, such as albumin. The opened maleimide may also cooperate with the divalent group X and the solubilizing moiety S to achieve improved stability and therapeutic efficacy.

In a preferred embodiment, Y is a divalent group derived from maleimides and derivatives thereof, such as opened hydrolyzed maleimides, preferably a divalent group represented by any of the following formulae (26a) to (26c):

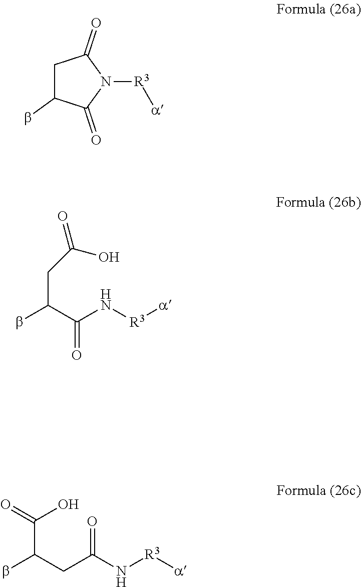

wherein,
R³ represents —(CH₂)ₙ₉—(C=A)ₙ₁₁- or —(CH₂CH₂O)ₙ₁₀—(C=A)ₙ₁₁-, preferably —(CH₂)ₙ₉—(C=A)ₙ₁₁-, wherein,
n9 is 1 to 6, preferably 1 or 2, more preferably 1,
n10 is 1 to 6, preferably 1,
n11 is 0 or 1, preferably 1, and
A is O or S, preferably O;
wherein the methylene carbon atom is covalently attached to the nitrogen atom of formulae (26a)-(26c) and the carbonyl or thiocarbonyl-carbon is covalently attached to T;
β indicates covalent attachment to Ab; and
α′ indicates covalent attachment to T.

In a more preferred embodiment, Y is represented by formula (26b) or (26c), wherein R³ is preferably a group represented by the formula —(CH₂)ₙ₉—(C=A)ₙ₁₁- in which n9 is 1 or 2, n11 is 1 and A is O. Most preferably, R³ is —CH₂—C═O—.

### (4) Antitumor Compound

In Formula (1), D is an antitumor compound (the drug moiety in the antibody-drug conjugate), and is conjugated to the anti-BCAM antibody via the linker. The antitumor compound is a compound having an antitumor effect and a substituent group or a partial structure allowing connection to the linker. When a part or whole linker is cleaved in tumor cells, the antitumor compound is released to exhibit the antitumor effect. As the linker is cleaved at a connecting position to the drug, the antitumor compound can be released in an unmodified structure to exhibit its intrinsic antitumor effect.

In Formula (1), n may be from 1 to 10. In another embodiment, n may be from 3 to 8. In one embodiment, n may be about 4. In another embodiment, n may be 8. In one embodiment, n may be from 3 to 5. In one embodiment, n may be from 7 to 9. In addition, in Formula (1), m may be from 1 to 5. In one embodiment, m may be 1.

The drug (the antitumor compound) to antibody (the anti-BCAM antibody) ratio (DAR) in the antibody-drug conjugate may be calculated by multiplying n by m in Formula (1). However, it is also understood the drug antibody ratio will often be an average value when used to describe a sample containing many molecules, due to some degree of inhomogeneity, typically associated with the conjugation step. The average drug antibody ratio, for instance, may be in the range of about 1 to about 10, and may be about 1, 1.5, 2, 2.5, 3, 3.5, 4, 4.5, 5, 5.5, 6, 6.5, 7, 7.5, 8, 8.5, 9, 9.5 or 10. In some aspects, the DAR may be from about 3 and about 8, and may be typically about 3, 3.5, 4, 4.5, 5, 5.5, 6, 6.5, 7, 7.5, 8, 8.5 or 9. In some aspects, the DAR may be about 4. In some aspects, the DAR may be about 8. In some aspects, at least 50% of a sample by weight may be compound having the average DAR±2, and preferably at least 50% of the sample may be a conjugate that contains the average DAR±1. In some aspects, a DAR of ‘about n’ means that the measured value for DAR is within ±20% of n (e.g., between 80% of n and 120% of n).

In one embodiment, the antitumor compound may include toxins targeting tubulin filaments, toxins targeting DNA, toxins targeting RNA, nanocarriers, protein toxins, etc. The toxins targeting tubulin filaments, for example, may include auristatin, maytansinoid, taxoid, etc. Auristatin is a synthetic antineoplastic agent derived from the natural product dolastatin 10, and is 100 to 1000 times more toxic than Doxorubicin, a conventional cancer chemotherapy medication. The dolastatin 10 is a nonspecific toxic agent, and because of this reason, it does not use as an antitumor compound in antibody-drug conjugate design. However, the synthetic analogues of this class of drug such as monomethyl auristatin E (MMAE) and monomethyl auristatin F (MMAF or AF) may be used as the antitumor compound in the antibody-drug conjugate. In particular, monomethyl auristatin E (MMAE) is an antimitotic agent that exerts its action by blocking the tubulin polymerization process resulting in cell-cycle arrest and apoptosis. The main function of monomethyl auristatin F (MMAF or AF) is the same as that of monomethyl auristatin E (MMAE) while MMAF is more hydrophilic and has a lower aggregation tendency to show lower systemic toxicity than MMAE.

Maytansinoid is a microtubule-disrupting agent isolated from the maytansine, a benzoansamacrolide. They inhibit tubulin polymerization resulting in mitotic arrest and cell death. Maytansinoids, for instance, include ansamitocin, mertansine/emtansine (DM1), and ravtansine/soravtansine (DM4).

Taxoid is an antineoplastic drug promoting microtubule assembly and inhibiting disassembly. Taxoid, for example, includes paclitaxel (Taxol) and docetaxel (Taxotere).

Toxins targeting DNA may include compounds modifying DNA bases, intercalating between bases, or forming crosslinks in DNA such as DNA alkylators cyclophosphamide, melphalan and chlorambucil. Toxins targeting DNA may also include antimetabolites, which mimics normal cellular molecules and interferes with DNA replication. These agents are usually DNA antagonists such as pyrimidine analogs 5-fluorouracil (5-FU), fluoxuridine, gemcitabine and purine analogs to block nucleotide metabolism pathways. Toxins targeting DNA, for instance, may include calicheamicins, CC-1065 analogs, and duocarmycins.

Toxins targeting RNA, for instance, may include amatoxins.

Nanocarriers, which may be in the size of 1-1000 nm, deliver therapeutic agents at the disease site for the treatment of various diseases. They may protect the drug from premature degradation and interacting with the biological environment, enhance the absorption of drugs into a selected tissue, and/or improve intracellular penetration. The nanocarriers, for instance, may include liposomal nanocarriers and non-liposomal nanoparticles. Liposomes are small, spherical vesicles of one (unilamellar) or more (multilamellar) phospholipid bilayers, which enclose an aqueous internal compartment. With the ability to carry both hydrophilic macromolecules in their aqueous core and lipophilic macromolecules within their lipid membranes, liposomes can act as suitable carriers for a broad range of drugs. Their ability to self-assemble, biocompatibility and large carrying capacity all contribute to their popularity as nanomedicine agents. Nanoparticles can be also made from a broad range of biological and synthetic materials and form a diverse array of structures, and such may include polymeric nanoparticles which are solid particles, or particulate dispersions of particles that can take the form of nanocapsules or nanospheres. Nanospheres are spherical polymer matrices on which a therapeutic antitumor compound is equally distributed throughout, whereas in nanocapsules, the therapeutic cargo is encapsulated by a polymer membrane. Such nanocarriers including one or more of various types of antitumor compounds therein may be used as the antitumor compound in the antibody-drug conjugate.

Protein toxin which is conjugated to the anti-BCAM antibody may be also used as the antitumor compound in the antibody-drug conjugate. It may be produced from bacteria and plants, and may consist of two moieties, one target on the cell surface and the other enters the cytosol, inhibiting protein synthesis. Bacterial toxins, for instance, may include Shiga toxin, Shiga-like toxin, Pseudomonas exotoxin, diphtheria toxin and cholera toxin. Plant toxins, for example, may include ricin, modeccin, abrin, volkensin and viscumin.

In one embodiment, the antitumor compound (D) may be selected from DNA-alkylating agents, topoisomerase inhibitors, RNA-polymerase II inhibitors, DNA-cleaving agents, antimitotic agents or microtubule disruptors, anti-metabolites, Kinesin spindle protein inhibitors, kinase inhibitors, nicotinamide phosphoribosyl transferase inhibitors, matrix metallopeptidase 9 inhibitors, phosphatase inhibitors, or radioisotopes and/or pharmaceutically acceptable salts thereof; if more than one D is present, each D is independently selected from the aforementioned compounds.

In one embodiment, the antitumor compound may be cytotoxic agents including (i) alkylating agents, such as aziridines (e.g., diaziquone, mytomycin and thiotepa), nitrogen mustards (e.g., mannomustine, mustine [mechlorethamine], aniline mustard, bendamustine, benzoic acid mustard, chlorambucil, C6-galactose mustard, melphalan, ossichlorin [nitromin], prednimustine, uramustine, nitrogen mustard carbamates [e.g., estramustine], and oxazaphosphorines [e.g., cyclophosphamide, ifosfamide, mafosfamide, and trofosfamide]), nitrosoureas (e.g., carmustine, fotemustine, lomustine, nimustine, N-nitroso-N-methylurea, ranimustine, semustine and streptozotocin), platinum-containing compounds (e.g., cisplatin, carboplatin and oxaliplatin), alkylsulfonates (e.g., busulfan, mannosulfan and treosulfan), hydrazines (e.g., dacarbazine and procarbazine), imidazotetrazines (e.g., mitozolomide and temozolomide), and triazines (e.g., hexamethylmelamine [altretamine]); (ii) cytotoxic antibiotics, such as anthracyclines (e.g., aclarubicin, daunorubicin, doxorubicin, epirubicin, idarubicin, mitoxantrone, pirarubicin and valrubicin), actinomycins (e.g., actinomycin D), bleomycins (e.g., bleomycins A2 and B2), mitomycins (e.g., mitomycin C), and plicamycins; (iii) anti-metabolites, such as anti-folates (e.g., aminopterin, methotrexate and pemetrexed), deoxynucleoside analogs (e.g., 5-azacytidine [azacitidine], 5-aza-2′-deoxycytidine [decitabine], cladribine, clofarabine, cytarabine, decitabine, fludarabine, gemcitabine, nelarabine and pentostatin), fluoropyrimidines (e.g., 5-fluorouracil, 5-fluoro-5′-deoxyuridine [doxifluridine] and capecitabine), and thiopurines (e.g., thioguanine, azathioprine and mercaptopurine); (iv) anti-microtubule agents, such as dolastatins (e.g., dolastatin 15), epothilones (e.g., epothilones A-F), taxanes (e.g., paclitaxel and docetaxel), vinca alkaloids (e.g., vinblastine, vincristine, vindesine, vinflunine and vinorelbine), colchicine, nocodazole, podophyllotoxin and rhizoxin; (v) histone deacetylase inhibitors, such as trichostatins (e.g., trichostatin A), romidepsin and vorinostat; (vi) kinase inhibitors, such as curcumin, cyclocreatine, deguelin, fostriecin, hispidin, tyrphostins (e.g., tyrphostins AG 34 and AG 879), bortezomib, erlotinib, gefitinib, imatinib, vemurafenib and vismodegib; (vii) topoisomerase I inhibitors, such as SN-38, exatecan, camptothecin, irinotecan and topotecan; (viii) topoisomerase II-targeting agents, such as topoisomerase II poisons (e.g., etoposide, tafluposide, teniposide, doxorubicin and mitoxantrone) and topoisomerase II inhibitors (e.g., novobiocin, merbarone and aclarubicin); (ix) DNA or RNA synthesis inhibitors, such as 3-amino-1,2,4-benzotriazine 1,4-dioxide, cytosine β-D-arabinofuranoside, 5,6-dichlorobenzimidazole 1-β-D-ribofuranoside, ganciclovir and hydroxyurea; (x) protein synthesis inhibitors, such as homoharringtonine; (xi) cell growth and differentiation regulators, such as retinoids (e.g., all-trans retinol [vitamin A], 11-cis retinol, all-trans retinal [vitamin A aldehyde], 11-cis retinal, all-trans retinoic acid [tretinoin], 9-cis-retinoic acid [alitretinoin], 11-cis retinoic acid, 13-cis-retinoic acid [isotretinoin], all-trans retinyl esters, etretinate, acitretin, adapalene, bexarotene and tazarotene); (xii) cell proliferation inhibitors, such as mTOR inhibitors (e.g., rapamycin [sirolimus]), apigenin, cholecalciferol (vitamin D3) and sex hormone-binding globulin; (xiii) apoptosis inducers, such as 17-allylamino-17-demethoxygeldanamycin, melatonin, mevinolin, psoralen, thapsigargin and troglitazone; (xiv) alkaloids such as maytansinoids (e.g. DM1, DM2, DM3, DM4, maytansine and ansamitocins), cryptophycins (e.g. cryptophycin 1 and cryptophycin 8), eleutherobin, discodermolide, bryostatins, auristatins (e.g. monomethyl auristatin E, monomethyl auristatin F), tubulysins, cephalostatins, pancratistatin, sarcodictyin; spongistatin; demecolcine; epipodophyllins (e.g. 9-aminocamptothecin, crisnatol, daunomycin, etoposide, etoposide phosphate, metabolites of irinotecan such as SN-38, mitoxantrone, novantrone, retinoic acids (retinols), teniposide, topotecan, 9-nitrocamptothecin (RFS 2000)), mitomycins (e.g. mitomycin C); and analogs, derivatives and salts thereof.

In another embodiment, the antitumor compound may be agents that stimulate the immune system, including without limitation including (i) agonists/activators of tumor necrosis factor receptor superfamily member 4 (TNFRSF4, OX40 or CD134); (ii) agonists/activators of TNFRSF member 5 (TNFRSF5 or CD40); (iii) agonists/activators of TNFRSF member 9 (TNFRSF9, 4-1BB or CD137); (iv) agonists/activators of TNFRSF member 18 (TNFRSF18, glucocorticoid-induced TNFR-related protein [GITR] or CD357); (v) agonists/activators of toll-like receptors (TLRs); and analogs, derivatives, fragments and salts thereof.

In another embodiment, the antitumor compound may be agents that block immune checkpoints including (i) inhibitors of cytotoxic T lymphocyte-associated protein 4 (CTLA-4) receptor or ligands thereof; (ii) inhibitors of killer cell immunoglobulin-like receptors (KIRs) or ligands thereof; (iii) inhibitors of lymphocyte activation gene 3 (LAG-3) receptor or ligands thereof; (iv) inhibitors of indoleamine 2,3-dioxygenase (IDO or IDO1), such as indoximod (1-methyl-D-tryptophan or NLG-8189), NLG-919, INCB024360, α-methyl-tryptophan, β-carboline (9H-pyrido[3,4-b]indole or norharmane), and cyclooxygenase 2 (COX-2) inhibitors (e.g., coxibs [such as apricoxib, celecoxib, etoricoxib, lumiracoxib, parecoxib, rofecoxib and valdecoxib], which down-regulate the expression of IDO); and analogs, derivatives, fragments and salts thereof.

In another embodiment, the antitumor compound may be angiogenesis inhibitors, including inhibitors of vascular endothelial growth factors (VEGFs) (e.g., squalamine) or receptors therefor (VEGFRs) (e.g., axitinib, pazopanib, sorafenib and sunitinib), inhibitors of platelet-derived growth factors (PDGFs) (e.g., squalamine) or receptors therefor (PDGFRs) (e.g., axitinib, pazopanib, sorafenib and sunitinib), inhibitors of fibroblast growth factors (FGFs) (e.g., squalamine) or receptors therefor (FGFRs), inhibitors of angiopoietins or receptors therefor, inhibitors of integrins (e.g., ALG-1001 and JSM-6427), anecortave (anecortave acetate), angiostatin (e.g., angiostatin K1-3), αVβ3 inhibitors (e.g., etaracizumab), berberine, bleomycins, borrelidin, carboxyamidotriazole, cartilage-derived angiogenesis inhibitors (e.g., chondromodulin I and troponin I), castanospermine, CM101, cyclopropene fatty acids (e.g., sterculic acid), adifluoromethylornithine, endostatin, everolimus, fumagillin, genistein, interferon-α, interleukin-12, itraconazole, linomide, matrix metalloproteinase (MMP) inhibitors (e.g., batimastat, cipemastat, ilomastat, marimastat, prinomastat, rebimastat, tanomastat, and tetracyclines [e.g., doxycycline, incyclinide and minocycline]), 2-methoxyestradiol, pigment epithelium-derived factor (PEDF), platelet factor-4, PPAR-γ agonists (e.g., thiazolidinediones, such as ciglitazone, lobeglitazone, netoglitazone, pioglitazone, rivoglitazone, rosiglitazone and troglitazone), prolactin, sphingosine-1-phosphate inhibitors, squalene, staurosporine, angiostatic steroids (e.g., tetrahydrocortisol) plus heparin, stilbenoids, suramin, SU5416, tasquinimod, tecogalan, tetrathiomolybdate, thalidomide and derivatives thereof (e.g., lenalidomide and pomalidomide), thiabendazole, thrombospondins (e.g., thrombospondin 1), TNP-470, tranilast, Withaferin A, and analogs, derivatives, fragments and salts thereof.

In another embodiment, the antitumor compound may be agents including (i) drug-efflux pump inhibitors, such as P-glycoprotein inhibitors (e.g., mifepristone and verapamil); (ii) cell adhesion inhibitors, such as cimetidine; (iii) Golgi apparatus disruptors, such as brefeldins (e.g., brefeldin A); (iv) ionizing radiation, such as X-ray; (v) radiation sensitizers of tumor cells, such as poly(ADP-ribose) polymerase (PARP) inhibitors (e.g., 4-amino-1,8-naphthalimide), berberine and indomethacin; (vi) enhancers of cell survival after treatment with cytotoxic drugs or radiation, such as pifithrin-a; (vii) vaccines, such as those that stimulate the immune system to recognize proteins produced by tumor cells and thereby to attack tumor cells; and analogs, derivatives and salts thereof.

The drugs used herein may also include radioisotopes as replacement of atoms contained therein. Examples of radioisotopes (radionuclides) are for instance ³H, ^{U}C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹⁷⁷Lu, ¹⁸⁶Re, ¹⁸⁸Re, ²¹¹At, ²¹²Bi, ²¹³Bi or ²²⁵Ac. Radioisotope labeled drugs can be used in targeted imaging experiments, or in targeted treatments (Wu et al Nat. Biotech. 2005, 23, 1137-1146). Imaging can be carried out by known computer tomography techniques such as Positron Emission Tomography (PET) or Single-Photon Emission Computed Tomography (SPECT); for a review of these techniques and applications see, e.g., Shankar Vallabhajosula (ed.), Molecular Imaging, Radiopharmaceuticals for PET and SPECT, Springer Verlag or Lucia Martiniova et al., Gallium-68 in Medical Imaging, Current Radiopharmaceuticals, 2016, 9, 187-207. In some aspects, the conjugate of the present disclosure can thus be used for diagnosing the progression and/or state of cancer.

In one embodiment, the antitumor compound (D) is selected from DNA-alkylating agents, topoisomerase inhibitors, RNA-polymerase II inhibitors, DNA-cleaving agents, antimitotic agents or microtubule disruptors, anti-metabolites, Kinesin spindle protein inhibitors, kinase inhibitors, nicotinamide phosphoribosyl transferase inhibitors, matrix metallopeptidase 9 inhibitors, phosphatase inhibitors, or radioisotopes and/or pharmaceutically acceptable salts thereof; if more than one D is present, each D is independently selected from the aforementioned compounds. Nonetheless, if more than one (D) is present in the compound of Formula (I) (n>1 and/or m>1), it is preferred that the multiple moieties (D) are identical to each other.

In a preferred embodiment, the antitumor compound (D) is selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof; if more than one (D) is present, each (D) is independently selected from the aforementioned compounds. Most preferably, the multiple moieties (D) are identical to each other.

It is more preferred that the antitumor compound (D) is selected from auristatin, MMAF, exatecan, maytansine, DM1 and DM4, and even more preferred that the antitumor compound is selected from auristatin and DM1. If more than one (D) is present, each (D) is independently selected from the aforementioned compounds. Most preferably, the multiple moieties (D) are identical to each other.

### (5) Exemplary Antibody-Drug Conjugates

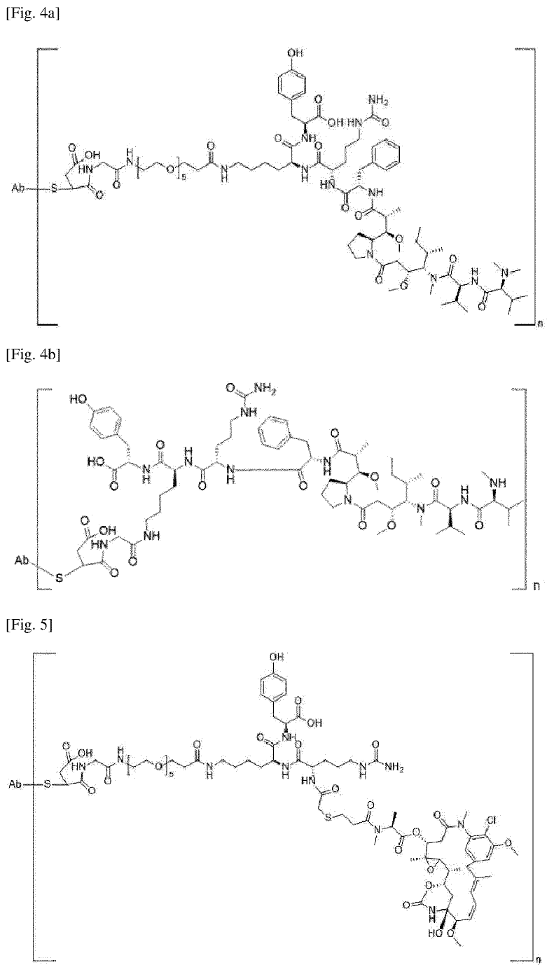

In one embodiment, the antibody-drug conjugate may have the structure as shown in FIG. 4a (Ab-Linker-AF). Here, Ab was prepared as explained in Example 1, and was conjugated to AF. In addition, n may be from 1 to 10. In one embodiment, n may be about 4. In another embodiment, n may be about 8.

In another embodiment, the antibody-drug conjugate may have the structure as shown in FIG. 4b (Ab-Linker-MMAF). Here, Ab was prepared as explained in Example 1, and was conjugated to MMAF. In addition, n may be from 1 to 10. In one embodiment, n may be about 4. In another embodiment, n may be about 8.

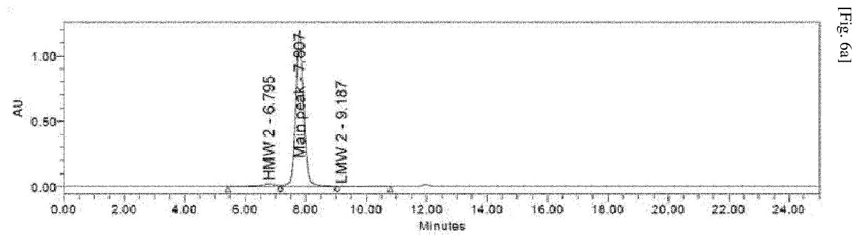

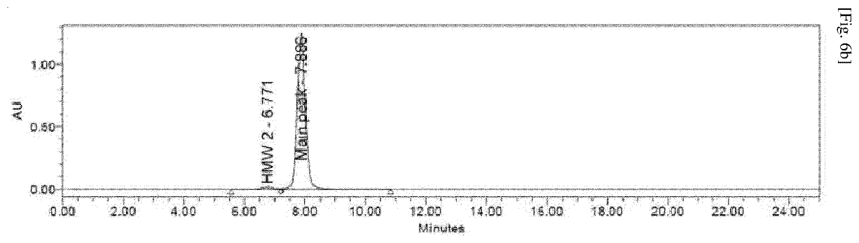

In yet another embodiment, the antibody-drug conjugate may have the structure as shown in FIG. 5 (Ab-Linker-DM1). Here, Ab was prepared as explained in Example 1, and was conjugated to DM1. In addition, n may be from 1 to 10. In one embodiment, n may be about 4. In another embodiment, n may be about 8.

### (6) Preferred Antibody-Drug Conjugates

In a preferred embodiment, the antibody-drug-conjugate of the disclosure is represented by Formula (17), or Formula (18):

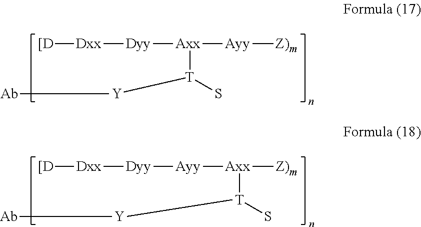

wherein Axx is an amino acid selected from Glu, Apa, Aaa, Dap, Dab, Lys, Orn, Ser, Ama and homo-Lys, with the proviso that Axx in Formula (17) is not in the (D) configuration,
wherein Ayy in Formula (17) is an amino acid selected from Phe, homo-Phe, Ala, Trp, Phg, Leu, Val, Tyr, homo-Tyr, Tyr(OR₁) and homo-Tyr(OR₁) wherein R₁ is —(CH₂ CH₂O)ₙ₁—R₂, wherein R₂ is a hydrogen atom or a methyl group and n1 is an integer of 2 to 24,
wherein Ayy in Formula (18) is an amino acid selected from Phe, homo-Phe, Ala, Trp, Phg, Leu, Val, Tyr and Ser, with the proviso that Ayy in Formula (18) is not in the (D) configuration,
wherein Dxx is a single covalent bond or an amino acid having a hydrophobic side chain,
wherein Dyy represents a single covalent bond, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein Y is a divalent group comprising one or more atoms selected from C, N, O, P and S, preferably a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of Ayy or Axx selected from —OH and —N(H)(R), wherein R represents a hydrogen atom, an alkyl group or a cycloalkyl group, and
wherein Ab, D, m and n are as defined in Formula (1).

In Formulae (17) and (18), it is preferred that at least one, e.g., one, two, three, four, five, six, seven or eight, of Axx, Ayy, Dxx, Dyy, D, Z, m and T is/are defined as follows:
(a) Axx is an amino acid selected from Dap, Dab, Lys, Om and homo-Lys,
(b) Ayy in Formula (17) is an amino acid selected from Phe, homo-Phe, Tyr, homo-Tyr, Tyr(OR₁) and homo-Tyr(OR₁),
(c) Ayy in Formula (18) is an amino acid selected from Phe, home-Phe or Ser,
(d) Dxx is a moiety derived from an amino acid selected from Phe, Val, Tyr, homo-Phe and Ala,
(e) Dyy is a covalent bond or a moiety derived from an amino acid selected from Arg, Lys, Cit, Orn, Dap and Dab,
(f) D is an antitumor compound selected from AF, MMAF, exatecan, maytansine, DM1 and DM4, preferably an antitumor compound selected from auristatin and DM1,
(g) Z is —OH or —NH₂,
(h) T is represented by Formula (9′):

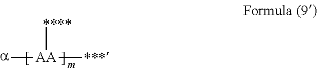

wherein each AA is independently a moiety comprising a trifunctional amino acid,
wherein α indicates covalent attachment to Y,
wherein m is as defined in claim 1,
if m is 1, the side chain of the trifunctional amino acid is covalently attached to S or Axx, the C-terminus being covalently attached to the other moiety S or Axx, respectively,
if m is 2, 3, 4 or 5, **** indicates covalent attachment to Axx, and ***′ indicates covalent attachment to S via the C-terminus of the chain of AA groups,
(i) m is 2 and T is represented by Formula (10′):

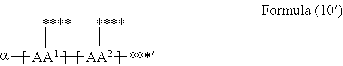

wherein each AA¹ and AA² is independently a moiety comprising a trifunctional amino acid,
wherein α indicates covalent attachment to Y,
wherein **** indicates covalent attachment to Axx, and ***′ indicates covalent attachment to S,
(j) m is 1 and T is represented by Formula (11′):

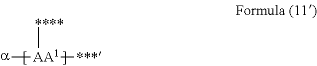

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(k) m is 1 and T is represented by Formula (12′), or Formula (13′):

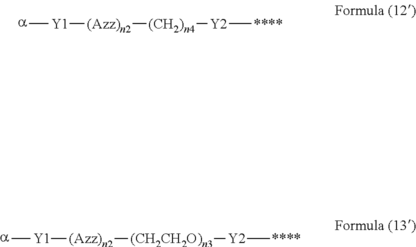

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to Axx.

In Formulae (17) and (18), it is more preferred that at least one, e.g., one, two, three, four, five, six, seven, eight or nine, of Axx, Ayy, Dxx, Dyy, D, Z, m and T is/are defined as follows:
(a) Axx is Lys,
(b) Ayy in Formula (17) is Tyr,
(c) Ayy in Formula (18) is Phe or Ser,
(d) Dxx is Phe or Val,
(e) Dyy is Arg or Cit,
(f) D is an antitumor compound selected from AF, MMAF, exatecan, maytansine, DM1 and DM4, preferably an antitumor compound selected from auristatin and DM1,
(g) Z is —OH or —NH₂,
(h) m is 1 and T is represented by Formula (11′):

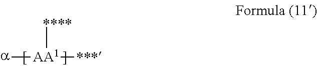

wherein AA¹ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(i) m is 1 and T is represented by Formula (12′), or Formula (13′):

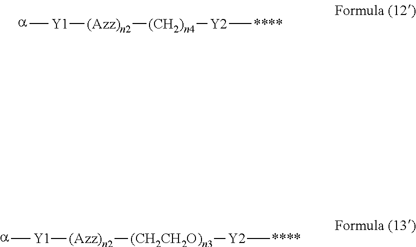

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to Axx.

In Formulae (17) and (18), it is even more preferred that at least one, e.g., one, two, three, four, five, six, seven, eight or nine, of Axx, Ayy, Dxx, Dyy, D, Z, m and T is/are defined as follows:
(a) Axx is Lys,
(b) Ayy in Formula (17) is Tyr,
(c) Ayy in Formula (18) is Phe or Ser,
(d) Dxx is Phe or Val,
(e) Dyy is Arg or Cit,
(f) D is a antitumor compound selected from AF and DM1,
(g) Z is —OH or —NH₂,
(h) m is 1 and T is represented by Formula (13′):

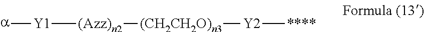

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is 0, 1, 2 or 3, preferably 0 or 1, most preferably 0,
wherein n3 is an integer of 2 to 24, more 4 to 12, such as 4 to 8,
wherein α indicates covalent attachment to Y, and
wherein **** indicates covalent attachment to Axx.

In a preferred embodiment, each Dxx-Dyy-Axx-Ayy in Formula (17) is independently selected from Arg-Lys-Phe wherein Dxx is a covalent bond, Arg-Lys-homo-Phe wherein Dxx is a covalent bond, Arg-Lys-Tyr wherein Dxx is a covalent bond, Cit-Lys-Phe wherein Dxx is a covalent bond, Cit-Lys-Tyr wherein Dxx is a covalent bond, Arg-Lys-homoTyr wherein Dxx is a covalent bond, Cit-Lys-homo-Tyr wherein Dxx is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homoPhe-Cit-Lys-Tyr.

In another preferred embodiment, each Dxx-Dyy-Ayy-Axx in Formula (18) is independently selected from Arg-Phe-Lys wherein Dxx is a covalent bond, Arg-Ser-Lys wherein Dxx is a covalent bond, Cit-Phe-Lys wherein Dxx is a covalent bond, Cit-Ser-Lys wherein Dxx is a covalent bond, Cit-homoPhe-Lys wherein Dxx is a covalent bond, Phe-Cit-Phe-Lys, homoPhe-Cit-Phe-Lys, and Phe-Arg-Phe-Lys.

In Formulae (17) and (18), it is even more preferred that at least one, e.g., one, two, three or four, of D, Z, m and T is/are defined as follows:
(a) D is a compound selected from a drug selected from AF, MMAF, exatecan, maytansine, DM1 and DM4,
(b) Z is —OH or —NH₂,
(c) m is 1 and T is represented by Formula (11′):

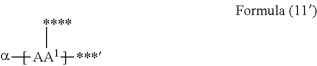

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(d) m is 1 and T is represented by Formula (12′), or Formula (13′):

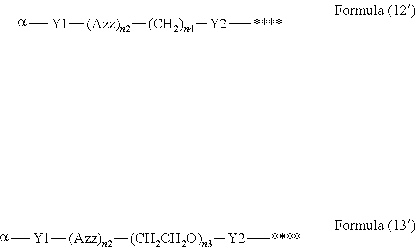

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to Axx.

In a more preferred embodiment, the antibody-drug-conjugate of the present disclosure is represented by one of the following formulae:

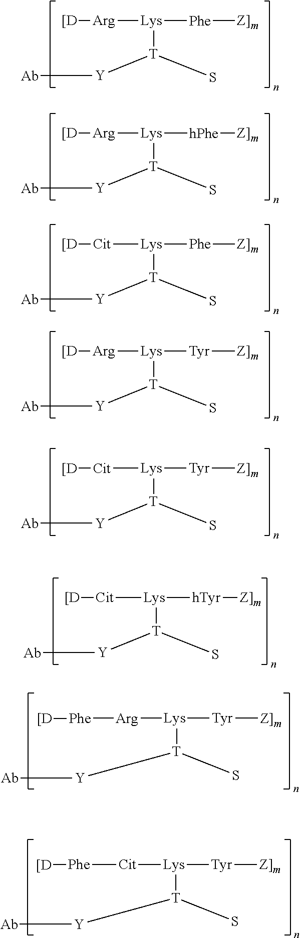

with the proviso that in the above formulae, Lys is not in the (D) configuration,
wherein Y is a divalent group comprising one or more atoms selected from C, N, O, P and S, preferably a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of an amino acid selected from —OH and —N(H)(R), wherein R represents a hydrogen atom, an alkyl group or a cycloalkyl group, and
wherein Ab, D, m and n are as defined in Formula (1).

In the above formulae, it is preferred that at least one, e.g., one, two, three or four, of D, Z, m and T is/are defined as follows:
(a) D is an antitumor compound selected from AF, MMAF, exatecan, maytansine, DM1 and DM4,
(b) Z is —OH or —NH₂,
(c) m is 2 and T is represented by Formula (10′):

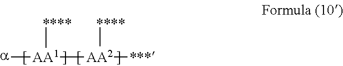

wherein each AA¹ and AA² is independently a moiety comprising a trifunctional amino acid,
wherein α indicates covalent attachment to Y,
wherein **** indicates covalent attachment to Lys, and ***′ indicates covalent attachment to S,
(d) m is 1 and T is represented by Formula (11′):

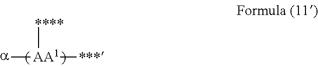

wherein AA′ is a moiety comprising a trifunctional amino acid,
wherein α indicates covalent attachment to Y,
wherein the side chain of the trifunctional amino acid is covalently attached to Lys or S, the C-terminus is covalently attached to the other moiety S or Lys, respectively,
(e) m is 1 and T is represented by Formula (12′), or Formula (13′):

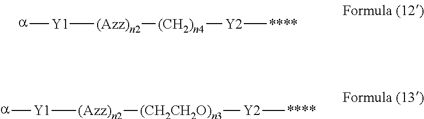

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to Lys.

In the above formulae, it is more preferred that D, Z, m and T are defined as follows:
(a) D is DM1,
(b) Z is —OH or —NH₂,
(c) m is 1 and T is represented by Formula (13′):

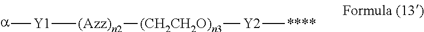

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein α indicates covalent attachment to Y, and
wherein **** indicates covalent attachment to Lys.

In the above formulae, D, Z, m and T is/are most preferably defined as follows:
(a) D is auristatin,
(b) Z is —OH,
(c) m is 1 and T is represented by Formula (13′):

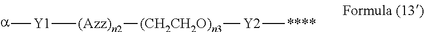

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is 0, 1, 2 or 3, preferably 0 or 1, most preferably 0,
wherein n3 is an integer of 2 to 24, preferably 4 to 12, such as 4 to 8,
wherein α indicates covalent attachment to Y, and
wherein **** indicates covalent attachment to Lys.

In a preferred embodiment, the antibody-drug-conjugate of the disclosure is represented by Formula (19), Formula (20), Formula (21), or Formula (22):

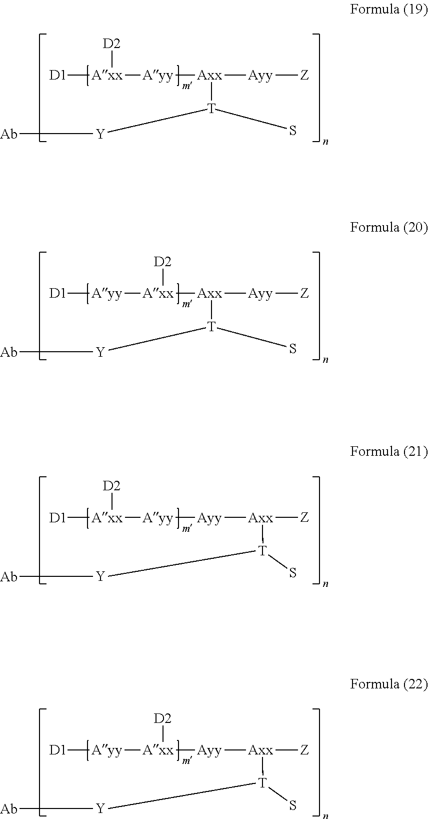

wherein Axx is a trifunctional amino acid, with the proviso that Axx in Formula (19) and Formula (20) is not an amino acid in the (D) configuration,
wherein Ayy is an amino acid selected from Phe, Ala, Trp, Tyr, Phg, Met, Val, His, Lys, Arg, Cit, Abu, Orn, Ser, Thr, Leu and Ile, or Ayy in Formula (19) and Formula (20) is an amino acid selected from homo-Tyr, homo-Phe, beta-Phe and beta-homo-Phe, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, with the proviso that Ayy in Formula (21) and Formula (22) is not an amino acid in the (D) configuration,
wherein each A″yy is independently an amino acid selected from Phe, Ala, Trp, Tyr, Phg, Met, Val, His, Lys, Arg, Cit, Abu, Orn, with the proviso that A″yy in Formula (20) and Formula (22) is not an amino acid in the (D) configuration,
wherein Y is a divalent group comprising one or more atoms selected from C, N, O, P and S, preferably a divalent group derived from a compound selected from maleimides, triazoles, hydrazones, carbonyl-containing compounds, and derivatives thereof, more preferably a divalent group derived from maleimides and derivatives thereof such as opened hydrolyzed maleimide derivatives,
wherein T is a tri-valent connecting group; if S is absent, T is a divalent connecting group,
wherein S is an atom or group that is optionally present to saturate a free valency of T,
wherein Z represents a group covalently bonded to the C-terminus of Ayy in Formula (19) and Formula (20) or to the C-terminus of Axx in Formula (21) or Formula (22), which is selected from —OH and —N(H)(R), wherein R represents a hydrogen atom, an alkyl group or a cycloalkyl group,
wherein D1 is an antitumor compound,
wherein m′ is (m−1), m being as defined in claim 1, with the proviso that m′ is not 0,
if m′ is 1, A″xx is a trifunctional amino acid with the proviso that A″xx in Formula (19) and Formula (21) is not an amino acid in the (D) configuration, and D2 is an antitumor compound,
if m′ is more than 1, each D2 is independently selected from a hydrogen atom and an antitumor compound, wherein multiple moieties D2 can be the same or different with the proviso that at least one D2 is not a hydrogen atom; if D2 is a hydrogen atom, A″xx is an amino acid with the proviso that A″xx in Formula (19) and Formula (21) is not an amino acid in the (D) configuration; if D2 is an antitumor compound, A″xx is a trifunctional amino acid with the proviso that A″xx in Formula (19) and Formula (21) is not an amino acid in the (D) configuration,
wherein Ab and n are as defined in Formula (1).

In Formula (19), Formula (20), Formula (21), and Formula (22), it is more preferred that at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Om and homo-Lys,
(b) Ayy in Formula (19) and Formula (20) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is independently an antitumor compound selected from AF, MMAF, exatecan, maytansine, DM1 and DM4, preferably an antitumor compound selected from auristatin and DM1,
(f) Z is —OH,
(g) m is 1 and T is represented by Formula (11′):

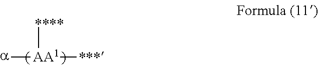

wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(h) m is 1 and T is represented by Formula (12′), or Formula (13′):

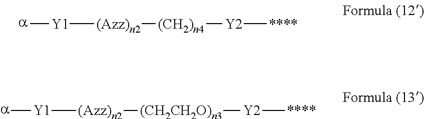

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5, preferably 0, 1, 2 or 3, more preferably 0 or 1, most preferably 0,
wherein n3 is an integer of 1 to 50, preferably 2 to 24, more preferably 4 to 12, such as 4 to 8,
wherein n4 is an integer of 1 to 50, preferably 2 to 20, more preferably 2 to 12,
wherein α indicates covalent attachment to Y, and
wherein **** indicates covalent attachment to Axx.

In Formula (19), Formula (20), Formula (21), and Formula (22), it is even more preferred that at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Om and homo-Lys,
(b) Ayy in Formula (17) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is auristatin,
(f) Z is —OH,
(g) m′ is 1 and T is represented by Formula (13′):

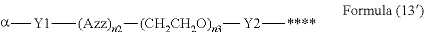

wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is 0, 1, 2 or 3, preferably 0 or 1, most preferably 0,
wherein n3 is an integer of 2 to 24, preferably 4 to 12, such as 4 to 8,
wherein α indicates covalent attachment to Y, and
wherein **** indicates covalent attachment to Axx.

In a more preferred embodiment, the antibody-drug-conjugate of the present disclosure is selected from the following compounds:

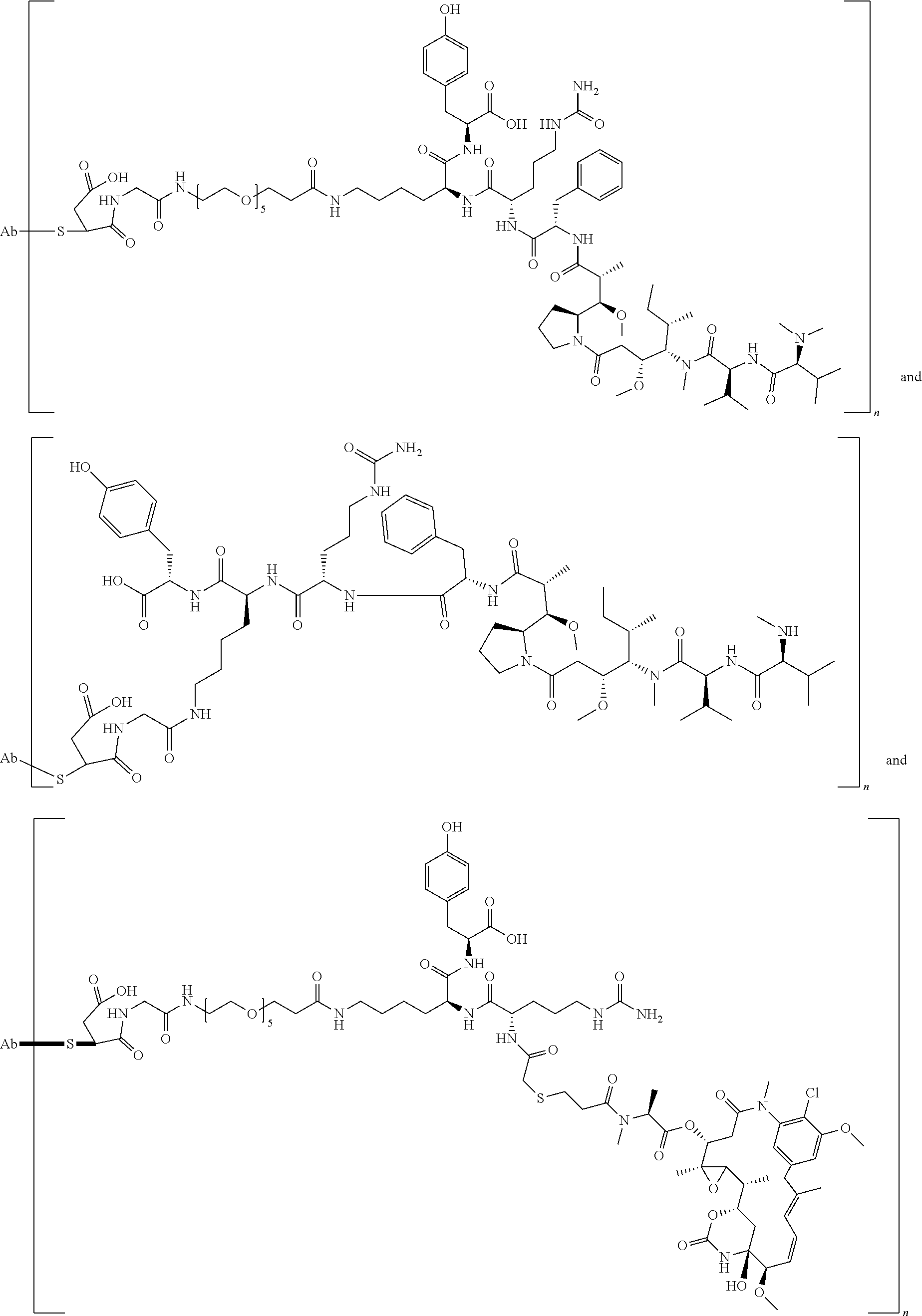

wherein,
Ab is an anti-BCAM antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6, and
n is 1 to 10, preferably 3 to 8.

In a preferred embodiment, in the above compounds, Ab is an anti-BCAM antibody or an antigen binding fragment thereof, wherein the heavy chain variable region comprises a sequence of SEQ ID NO: 7, and the light chain variable region comprises a sequence of SEQ ID NO: 8.

In one embodiment, in the above compounds, the opened maleimide attachment to Ab may be replaced by a closed maleimide attachment. In some instances, wherein n is more than 1, the compound may comprise a mix of (closed) maleimide derivatives and opened hydrolyzed maleimide derivatives attached to Ab, whereby preferably at least 50% of the attachments to Ab are opened hydrolyzed maleimide attachments, e.g., maleimide attachments represented by the Formula (26b) or (26c).

### 2. The Pharmaceutical Composition Comprising the Antibody-Drug Conjugate

The present disclosure also provides a pharmaceutical composition comprising the antibody-drug conjugate of the present disclosure and a pharmaceutically acceptable carrier. In one embodiment, the pharmaceutically acceptable carrier may be selected based on the particular antitumor compound used, and its concentration, stability and intended bioavailability, the disease, disorder or condition being treated with the composition, the subject, its age, size and general condition, and the route of administration. For instance, the antibody-drug conjugate of the present disclosure may be mixed with a solvent such as a sterilized liquid (including water and oil (oil derived from petroleum, animals, vegetables, or synthetic oil (e.g., peanut oil, soybean oil, mineral oil, sesame oil, etc.)), a saline, a dextrose aqueous solution, or a glycerol aqueous solution, and additives such as a moisturizer, an emulsifier, or a pH buffer, and the like, so as to prepare the pharmaceutical composition of the present disclosure. The pharmaceutically acceptable carrier for solid dosage forms may include sugars, starches, and other conventional substances including polysorbate, histidine, lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, tragacanth, microcrystalline cellulose, colloidal silicon dioxide, croscarmellose sodium, talc, magnesium stearate, and stearic acid. In addition, such solid dosage forms may be uncoated or may be coated by known techniques (e.g., to delay disintegration and absorption). In addition, the pharmaceutically acceptable carrier used in formulating liquid dosage forms for oral or parenteral administration, for instance, includes nonaqueous, pharmaceutically-acceptable polar solvents such as oils, alcohols, amides, esters, ethers, ketones, hydrocarbons and mixtures thereof, as well as water, saline solutions, dextrose solutions, electrolyte solutions, or any other aqueous, pharmaceutically acceptable liquid.

In one embodiment, the pharmaceutical composition is for treating cancer. The cancer may be a solid tumor e.g., may be a cancer of epithelial origin, or liquid tumour.

In an embodiment the cancer may be one or more selected from the group consisting of breast cancer, liver cancer, skin cancer, ovarian cancer, cervical cancer, prostate cancer, testicular cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, kidney cancer, head and neck cancer, hypopharyngeal squamous cell carcinoma, glioblastoma, neuroblastoma, endometrial cancer, and urothelial cell carcinoma.

### 3. Compound of Formula (25), Kit for the Modification of Anti-BCAM Antibodies, and Method for the Modification of Anti-BCAM Antibodies

In some aspects, the present disclosure relates to a compound, which can be used for the modification of vector molecules capable of interacting with target cells as described above, e.g., antibodies. The present disclosure thus relates to a compound represented by Formula (25):

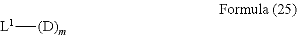

or a pharmaceutically acceptable salt thereof,
wherein D and m have the same meaning as described above with respect to the compound of Formula (1), and
wherein L¹ is a linker represented by Formula (26):

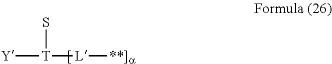

wherein L′, T, S, o and ** have the same meanings as described above with respect to the compound of Formula (I), and
wherein Y′ represents a moiety comprising a conjugation group capable of forming a covalent attachment to an anti-BCAM antibody or an antigen binding fragment thereof.

In one embodiment, Y′ is a moiety comprising a conjugation group selected from:
an optionally substituted maleimide, preferably capable of reacting with one or two thiol groups comprised in an anti-BCAM antibody or an antigen binding fragment thereof,
an optionally substituted haloacetamide, preferably capable of reacting with a thiol group comprised in an anti-BCAM antibody or an antigen binding fragment thereof,
an ester, preferably capable of reacting with the side chain of an amino acid comprised in an anti-BCAM antibody or an antigen binding fragment thereof such as an acyl halide, an N-hydroxy succinimide ester (structure shown below, left hand side, o indicates covalent attachment to the remainder of the compound of Formula (25)) or a phenolic ester (structure shown below, right hand side, o indicates covalent attachment to the remaining of the compound of Formula (25), each R being individually selected from H, F, NO₂ and CN),

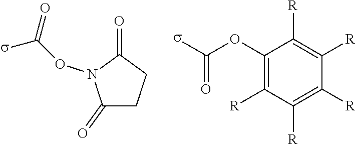

a carbonate, preferably capable of reacting with the side chain of an amino acid comprised in an anti-BCAM antibody or an antigen binding fragment thereof such as a haloformate or a carbonate comprising a leaving group such as a N-hydroxy succinimide or phenol derivative,
an isocyanate or isothiocyanate, preferably capable of reacting with the side chain of an amino acid comprised in an anti-BCAM antibody or an antigen binding fragment thereof,
an azide, preferably capable of reacting with an alkyne group comprised in an anti-BCAM antibody or an antigen binding fragment thereof (in which case, the anti-BCAM antibody or antigen binding fragment thereof can be modified by antibody engineering or other conjugation techniques to introduce an alkyne functionality),
an alkyne, preferably capable of reacting with an azide group comprised in an anti-BCAM antibody or an antigen binding fragment thereof (in which case, the anti-BCAM antibody or antigen binding fragment thereof can be modified by antibody engineering or other conjugation techniques to introduce an azide functionality), and
an amino group, e.g., a primary or secondary amino group, preferably capable of reacting with an anti-BCAM antibody or an antigen binding fragment thereof in the presence of an enzyme such as a transglutaminase.

In one embodiment, the present disclosure thus relates to a compound represented by Formula (25):

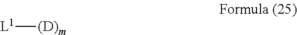

or a pharmaceutically acceptable salt thereof,
wherein D and m have the same meaning as described above with respect to the compound of Formula (1), and
wherein L² is represented by Formula (27), Formula (28), or Formula (29):

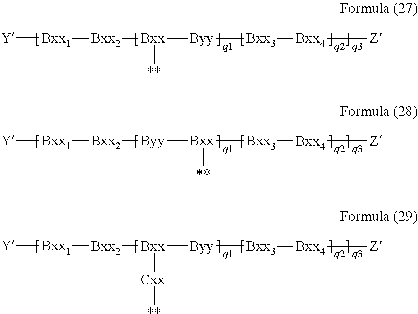

wherein Bxx, Byy, Cxx, Bxx1, Bxx2, Bxx3, Bxx4, q1, q2, q3, Z′ and ** have the same meanings as described above with respect to the compound of Formula (I), and
wherein Y′ has the same meaning as described above with respect to the compound of Formula (25).

In a preferred embodiment, the compound has a structure represented by Formula (30) or Formula (31):

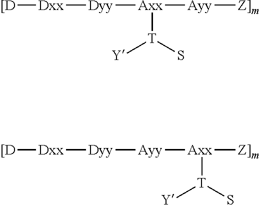

wherein D, Dxx, Dyy, Ayy, Axx, T, S, Z and m have the same meanings as in Formulae (17) and (18), and
wherein Y′ has the same meaning as described above with respect to the compound of Formula (25).

In a further preferred embodiment, each Dxx-Dyy-Axx-Ayy in Formula (30) is independently selected from Arg-Lys-Phe wherein Dxx is a covalent bond, Arg-Lys-homo-Phe wherein Dxx is a covalent bond, Arg-Lys-Tyr wherein Dxx is a covalent bond, Cit-Lys-Phe wherein Dxx is a covalent bond, Cit-Lys-Tyr wherein Dxx is a covalent bond, Arg-Lys-homoTyr wherein Dxx is a covalent bond, Cit-Lys-homo-Tyr wherein Dxx is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homoPhe-Cit-Lys-Tyr, and

each Dxx-Dyy-Ayy-Axx in Formula (31) is independently selected from Arg-Phe-Lys wherein Dxx is a covalent bond, Arg-Ser-Lys wherein Dxx is a covalent bond, Cit-Phe-Lys wherein Dxx is a covalent bond, Cit-Ser-Lys wherein Dxx is a covalent bond, Cit-homoPhe-Lys wherein Dxx is a covalent bond, Phe-Cit-Phe-Lys, homoPhe-Cit-Phe-Lys, and Phe-Arg-Phe-Lys.

In yet another preferred embodiment, the compound has a structure represented by one of the following formulae:

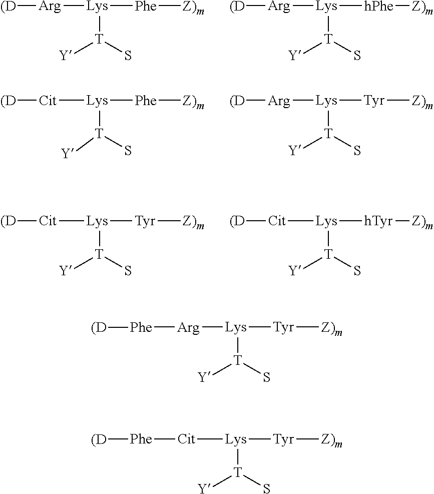

wherein D, T, S, Y′, Z and m have the same meanings as described above.

In some aspects, the present disclosure relates to a kit comprising the compound as described hereinbefore (i.e., the compound of Formula (25)) and a buffer, which can be used for the modification of anti-BCAM antibodies or antigen binding fragments thereof.

The compound and the buffer (together forming the kit) can be presented individually, e.g., in separate primary containers (which may be shipped to the customer in a single box), which can be stored for a prolonged period, without degradation. The compound and buffer can be formulated and portioned for a given amount of antibody or fragment thereof to be modified. In some aspects, the compound of the present disclosure is presented as a solid (e.g., as a lyophilized powder, or non-covalently adsorbed or covalently bound to a solid phase matrix as described further below), or as a solution in a suitable solvent, such as a water-miscible, polar aprotic solvent (e.g., DMF, DMSO), which can be mixed with the buffer shortly prior to antibody or antibody fragment modification.

The buffer to be used in the kit of the present disclosure is not particularly limited. Preferably, the buffer has a pH of 6.0 to 10, more preferably of 6.5 to 8.0. The buffer can be selected from, e.g., 2-bis(2-hydroxyethyl)amino acetic acid (Bicine), carbonate-bicarbonate, tris(hydroxymethyl)methylamino propane sulfonic acid (TAPS), 4-(2-hydroxyethyl)-1-piperazineethane sulfonic acid (HEPES).

In some aspects, the compound of the present disclosure can be used in a method for the modification of anti-BCAM antibodies or antigen binding fragments thereof. The method thus produces an antibody-drug-conjugate as described above.

In one embodiment, the method comprises the step of reacting (contacting) an anti-BCAM antibody or an antigen binding fragment thereof. For example, the reaction may be performed in a PBS buffer with 3 to 10%, preferably 5 to 10%, DMSO. An activator such as tris(2-carboxyethyl)phosphine (TCEP) may be used. Suitable reaction temperatures range from 5 to 40° C., preferably from 20 to 40° C. The reaction mixture can be purified by techniques known in the art, such as diafiltration techniques or gel permeation chromatography using a suitable solvent. Examples of suitable stationary phases for isolating the clean antibody-drug-conjugate include polyacrylamide gels, such as Bio-Gel® P-30 and crosslinked dextrans such as Sorbadex®, Zetadex® or Sephadex®. The method can be applied to any anti-BCAM antibody or antigen binding fragment thereof.

### 4. Preparation of the Compound of the Present Disclosure

In the following, methods are provided for the preparation of linkers, drug-linkers and antibody-drug-conjugates. The compounds of the present disclosure can be synthesized relying on standard organic chemistry reactions or Fmoc-based solid-phase peptide synthesis (SPPS), including in solution and on-resin peptide coupling and convergent strategies. The introduction of various maleimido-derivatives and subsequent chemoselective ligation to anti-BCAM antibodies or antigen binding fragments thereof is also exemplified below. The general strategies and methodology which can be used for preparing the compounds of the present disclosure are well-known to the person skilled in the art.

### 5. The Method for Treating Cancer by Administering the Pharmaceutical Composition

The present disclosure also provides a method for treating cancer comprising administering an effective amount of the antibody-drug conjugate of the present disclosure to a subject in need thereof.

In one embodiment, the effective amount means an amount of a therapeutic agent (e.g., antibody-drug conjugates) that is sufficient, when administered to a subject suffering from cancer to treat, alleviate, ameliorate, relieve, alleviate symptoms of, prevent, delay onset of, inhibit progression of, reduce severity of, and/or reduce incidence of cancer. For instance, the appropriate dosage of the antibodies, antigen binding fragments, or antibody-drug conjugates depend on various factors, such as the type of cancer to be treated, the severity and course of the cancer, the responsiveness of caner, previous therapy, patient's clinical history, and so on. The pharmaceutical composition can be administered one time or over a series of treatments lasting from several days to several months, or until a cure is effected or a diminution of cancer state is achieved (e.g., reduction in tumor size). Optimal dosing schedules can be calculated from measurements of drug accumulation in the body of the patient and will vary depending on the relative potency of an individual antibody, antibody fragment (e.g., antigen binding fragment), or antibody drug conjugates. In certain aspects, dosage is from 0.01 mg to 10 mg (e.g., 0.01 mg, 0.05 mg, 0.1 mg, 0.5 mg, 1 mg, 2 mg, 3 mg, 4 mg, 5 mg, 7 mg, 8 mg, 9 mg, or 10 mg) per kg of body weight per day, and can be given once or more daily, weekly, monthly or yearly. In certain aspects, the antibody, antibody fragment (e.g., antigen binding fragment), or antibody drug conjugate of the present disclosure is given once every two weeks or once every three weeks. The treating physician can estimate repetition rates for dosing based on measured residence times and concentrations of the drug in bodily fluids or tissues.

The pharmaceutical can be administered to the subject according to any methods. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. The pharmaceutical composition can be administered therapeutically (e.g., administered to treat an existing disease or condition), or for imaging purposes. However, the pharmaceutical composition can also be administered prophylactically (e.g., administered for prevention of a cancer, such as a blood cancer or a solid tumor).

The pharmaceutical of the present disclosure may also be administered together with an additional therapeutic agent (e.g., co-administration) such as a cytokine, steroid, chemotherapeutic agent, antibiotic, or radiation known in the art. Such additional therapies (e.g., prophylactic or therapeutic agents), which can be administered in combination with the pharmaceutical of the present disclosure may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the pharmaceutical composition of the present disclosure. The two or more therapies may be administered using the same or different modes of administration. The two or more therapies may be administered within the same patient visit.

### EXAMPLES

### Example 1

### Example 1.1—Preparation of the Anti-BCAM Antibody

As the anti-BCAM antibody, Ab1 (human IgG4 S228P isotype), Ab2 (human IgG1 isotype) and Ab3 (human IgG1 LALA isotype) were prepared under the same conditions as explained below.

Each of Ab1, Ab2 and Ab3 has the heavy chain variable region having the sequence of SEQ ID NO: 7, and the light chain variable region having the sequence of SEQ ID NO: 8. The heavy chain variable region comprises the VH CDR1 sequence of SEQ ID NO: 1, the VH CDR2 sequence of SEQ ID NO: 2, and the VH CDR3 sequence of SEQ ID NO: 3. The light chain variable region comprises the VL CDR1 sequence of SEQ ID NO: 4, the VL CDR2 sequence of SEQ ID NO: 5, and the VL CDR3 sequence of SEQ ID NO: 6. Ab1 has the heavy chain including a sequence of SEQ ID NO: 9, and the light chain including a sequence of SEQ ID NO: 11. Ab2 has the heavy chain including a sequence of SEQ ID NO: 10, and the light chain including a sequence of SEQ ID NO: 11. Ab3 has the heavy chain including a sequence of SEQ ID NO: 12, and the light chain including a sequence of SEQ ID NO: 11.

The sequence information referred to herein is as follows.

| | |
|---|---|
| SEQ ID NO: 1 | GFTFSDYYMS |
| (VH CDR1) | |
| SEQ ID NO: 2 | YTSSSGSTIYYADSVKG |
| (VH CDR2) | |
| SEQ ID NO: 3 | DITAFDI |
| (VH CDR3) | |
| SEQ ID NO: 4 | TGTSSDVGGYNYVS |
| (VL CDR1) | |
| SEQ ID NO: 5 | EVNKRPS |
| (VL CDR2) | |
| SEQ ID NO 6 | CSYAGSNNYV |
| (VL CDR3) | |
| SEQ ID NO: 7 | EVQLVQSGGGLVKPGGSERLSCAASGFTFSDYYMSWIRQAPGKGL |
| (Heavy chain | EWVSYTSSSGSTIYYADSVKGRFTISRDNAKNSLYLQMNSLRAED |
| variable region) | TAVYYCARDITAFDIWGQGTMVTVSS |
| SEQ ID NO: 8 | LPVLTQPPSASGSPGQSVTISCTGTSSDVGGYNYVSWYQQHPGKA |
| (Light chain | PKLMIYEVNKRPSGVPDRESGSKSGNTASLTVSGLQAEDEADYYC |
| variable region) | CSYAGSNNYVFGTGTKVTVL |
| SEQ ID NO: 9 | EVQLYQSGGGLVKPGGSLRLSCAASGETFSDYYMSWIRQAPGKGL |
| (Heavy chain | EWVSYTSSSGSTIYYADSVKGRFTISRDNAKNSLYLQMNSLRAED |
| of Ab1) | TAVYYCARDITAFDIWGQGTMVTVSSASTKGPSVFPLAPCSRSTSE |
| | STAALGCLVKDYFPEPVTVSWNSGALTSGVHTFPAVLQSSGLYSL |
| | SSVVTVPSSSLGTKTYTCNVDHKPSNTKVDKRVESKYGPPCPPCPA |
| | PEFLGGPSVELFPPKPKDTLMISRTPEVTCVVVDVSQEDPEVQFNW |
| | YVDGVEVHNAKTKPREEQFNSTYRVVSVLIVLHQDWLNGKEYK |
| | CKVSNKGLPSSIEKTISKAKGQPREPQVYTLPPSQEEMTKNQVSLT |
| | CLVKGFYPSDIAVEWESNGQPENNYKTTPPVLDSDGSFFLYSRLTV |
| | DKSRWQEGNVFSCSVMHEALHNHYTQKSLSLSLGK |
| SEQ ID NO: 10 | EVQLVQSGGGLVKPGGSLRLSCAASGETFSDYYMSWIRQAPGKGL |
| (Heavy chain | EWVSYTSSSGSTIYYADSVKGRFTISRDNAKNSLYLQMNSLRAED |
| of Ab2) | TAVYYCARDITAFDIWGQGTMVTVSSASTKGPSVFPLAPSSKSTSG |
| | GTAALGCLVNDYFPEPVTVSWNSGALTSGVHTFPAVLQSSGLYSL |
| | SSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEV |
| | KFNWYVDGVEVHNAKTKPREFQYNSTYRVVSVLTVLHQDWLNGK |
| | EYKCKVSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 11 | LPVLTQPPSASGSPGQSVTISCTGTSSDVGGYNYVSWYQQHPGKA |
| (Light chain | PKLMIYEVNKRPSGVPDRFSGSKSGNTASLTVSGLQAEDEADYYC |
| of Ab1, Ab2 or | CSYAGSNNYVFGTGTKVTVIGQPKAAPSVTLEPPSSEELQANKAT |
| Ab3) | LVCLISDFYPGAVTVAWKADSSPYKAGVETTTPSKQSNNKYAASS |
| | YLSLIPEQWKSHRSYSCQVTHEGSTVEKTVAPTECS |
| SEQ ID NO: 12 | EVQLVQSGGGLVKPGGSLRLSCAASGFTFSDYYMSWIRQAPGKGL |
| (Heavy chain | EWVSYTSSSGSTIYYADSVKGRETISRDNAKNSLYLQMNSLRAED |
| of Ab3) | TAVYYCARDITAFDIWGQGTMVTVSSASTKGPSVFPLAPSSKSTSG |
| | GTAALGCLVNDYFPEPVTVSWNSGALTSGVHTFPAVLQSSGLYSL |
| | SSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEV |
| | KFNWYVDGVEVENAKIKPREEQYNSTYRVVSVLIVLHQDWLNGK |
| | EYKCKYSNKALPAPIEKTISKAKGQPREPQVYTLPPSREEMTKNQV |
| | SLTCLVKGFYPSDIAVEWESNGQPENNYKTTPPVLDSDGSFFLYSK |
| | LTVDKSRWQQGNVFSCSVMHEALHNHYTQKSLSLSPGK |

For the production of anti-BCAM antibodies, their light and heavy chain variable regions were merged with each constant region of a human antibody, and they were cloned into each separate expression DNA vector. Prepared DNA vectors of light and heavy chain were transiently transfected into ExpiCHO-S™ cells (Thermofisher Scientific), thereafter, cultured in CO₂ incubator to express monoclonal antibodies with ExpiCHO® expression kit. After eight days of culture, the supernatant was collected from a culture medium with ultracentrifugation and clarified using depth or microfiltration.

Each harvested clarified culture medium was purified with Protein A resin using FPLC system to remove culture components and impurities. Specifically, the column which was pre-packed using the above resin was equilibrated with 50 mM Tris, 150 mM HCl, pH 7.5, thereafter, each harvest sample was loaded to the column to bind to Protein A resin. After the washing with equilibration buffer and high-salt-containing buffer, the purified sample was eluted using step-gradient elution by elution buffer (100 mM glycine of low pH buffer). After that, the pH of the elution sample was adjusted to 6.5, followed by loading to the cation exchange chromatography column equalized with 20 mM Histidine, pH 6.5. To elute the product with high resolution, a linear salt gradient was applied during 20 column volumes.

After the purification, 1 M Tris solution was added to the pooled sample to adjust to the pH range of 7.0-7.5 and transferred onto a 0.22 μm filter to remove the aggregates. Finally, the buffer of each product was changed to 1×PBS (Corning, phosphate-buffered saline with pH 7.4) by ultrafiltration and diafiltration with Amicon® ultracentrifugal filters (Millipore). For confirming the concentration of the final formulated products were analyzed by SoloVPE(Repligen) with A280/Extinction coefficient. To verify the product in terms of purity, samples were analyzed with the size-exclusion HPLC (SE-HPLC) and SDS-PAGE.

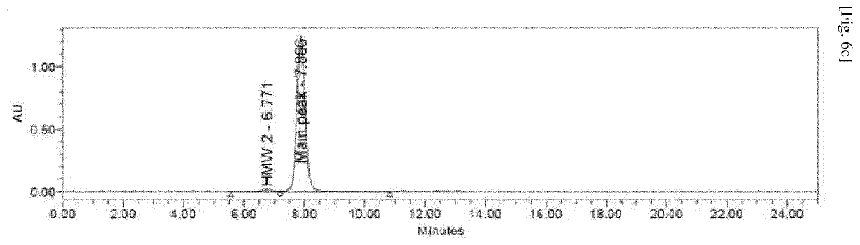

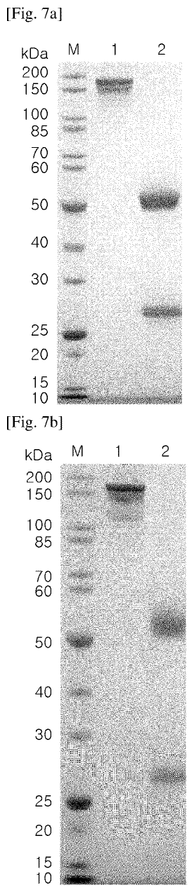

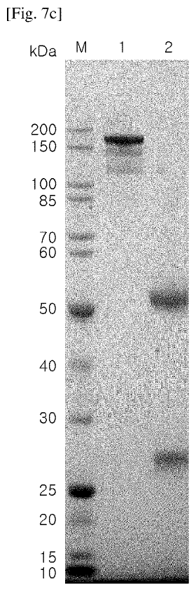

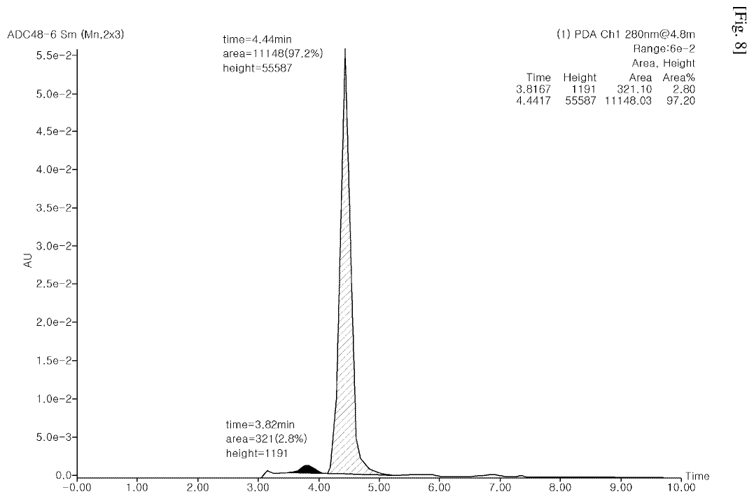

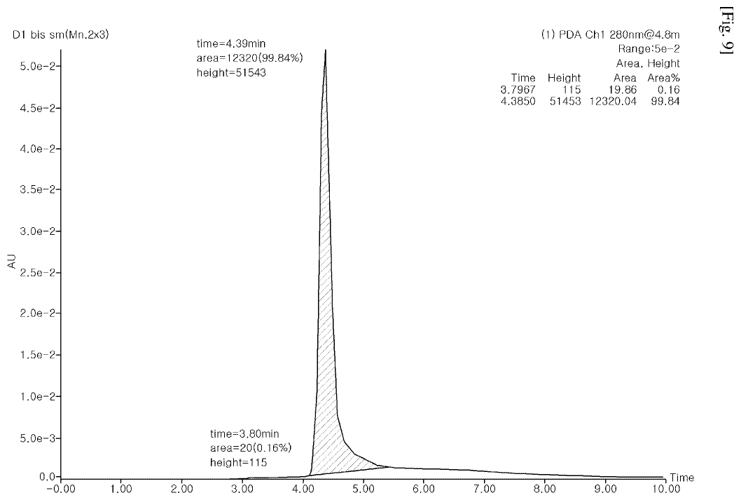

First, the samples were loaded on the size-exclusion column (TSKgel G3000SWXL 7.8×300 mm, TOSOH) and analyzed with phosphate-chloride buffer, and the chromatogram result of SE-HPLC were obtained as shown in FIG. 6 ((A): Ab1, (B): Ab2, (C): Ab3). In addition, the results of SDS-PAGE were obtained as shown in FIG. 7 ((A): Ab1, (B): Ab2, (C): Ab3). Here, the samples and their loading amounts in the 3 lanes, from left to right, were: M, marker, 5 μL; 1, non-reducted sample, 3 μg; 2, reducted sample, 3 μg.

The summarized concentration and purity analysis results of prepared anti-BCAM antibody (Ab1, Ab2, Ab3) were as follows.

TABLE 1A

| | Concentration | Purity |
|---|---|---|
| Sample | (mg/mL) | (by SE-HPLC, %) |
| Ab1 | 14.5 | 96.9 |
| Ab2 | 14.8 | 98.3 |
| Ab3 | 9.5 | 98.6 |

### Example 1.2—Binding Affinity of Ab1 and Ab2 to Human BCAM Recombinant Protein by Indirect ELISA

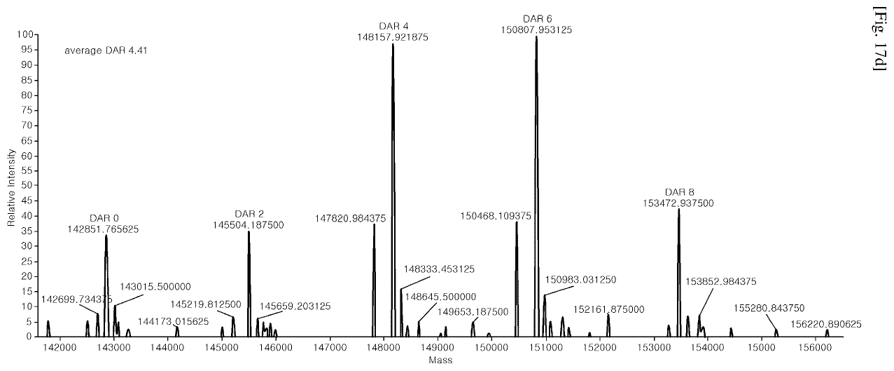

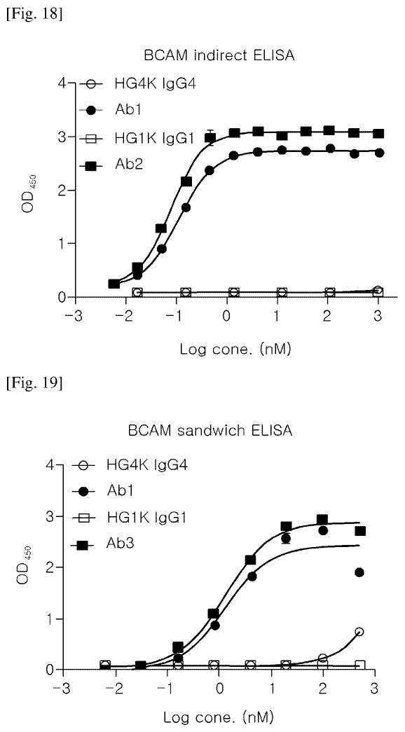

The binding affinity of Ab1 and Ab2 to human BCAM recombinant protein was evaluated using an indirect ELISA (Enzyme-Linked Immunosorbent Assay) method. Human BCAM (SinoBiological, USA) diluted in PBS to 25 nM, and 50 μL of prepared proteins was added to 96-well half plate (Costar, USA) and incubated overnight at 4° C. Next day, solution containing human BCAM was removed, and the plate was washed 3 times with 150 μL of PBS-T (0.1% Tween-20 in PBS). The plate coated with human BCAM was blocked with 150 μL of 3% BSA blocking buffer for 1 hr at 37° C. Blocking buffer was removed, and 50 μL of Ab1 and Ab2 (6 pM~1 μM, serial 3-fold dilution, 12 points), and HG4K IgG4 (SinoBiological, USA) and HG1K IgG1 (SinoBiological, USA) diluted with blocking buffer was added to each well and incubated for 2 hr at 37° C. The plate was washed 3 times with 150 μL of PBS-T, and secondary antibody (HRP-conjugated anti-human IgG Fc antibody; ThermoFisher Scientific, USA) diluted with blocking buffer (1:100,000) was added to each well and incubated for 1 hr at 37° C. The plate was washed 3 times with 150 μL of PBS-T, and μL of TMB-ELISA substrate solution (ThermoFisher Scientific, USA) was added to each well and incubated for 10 min at room temperature. The absorbance (Optical density at 450 nm, OD₄₅₀) was measured at 450 nm using Synergy H1 (BioTek, Winooski, VT). The graphs were generated as sigmoidal dose-response curves and EC₅₀ values were determined by nonlinear regression with a four-parameter logistic equation using GraphPad Prism software (GraphPad Software Inc., San Diego, CA, USA). The Ab1 and Ab2 were dose-dependently bound to human BCAM, and the EC₅₀ value for Ab1 and Ab2 was 0.112 nM and 0.078 nM, respectively. See FIG. 18. and Table 1B.

TABLE 1B
The binding affinity of Ab1 and Ab2 to human BCAM
recombinant protein by indirect ELISA (EC₅₀ value).

| Antibody | Ab1 | Ab2 |
|---|---|---|
| EC₅₀ (nM) | 0.112 | 0.078 |

### Example 1.3—Binding Affinity of Ab1 and Ab3 to Human BCAM Recombinant Protein by Sandwich ELISA

The binding affinity of Ab1 and Ab3 to human BCAM recombinant protein was evaluated using sandwich ELISA (Enzyme-Linked Immunosorbent Assay) method. Anti-His antibody (Biolegend, USA) diluted in PBS to 4 μg/mL was added to 96-well plate (ThermoFisher Scientific, USA) and incubated overnight at 4° C. Next day, solution containing anti-his antibody was removed, and the plate was washed 3 times with 300 μL of PBS-T (0.1% Tween-20 in PBS). The plate coated with anti-his antibody was blocked with 300 μL of 3% skim milk blocking buffer for 1 hr at room temperature. Human BCAM (SinoBiological, USA) diluted in blocking buffer to 25 nM, and 100 μL of prepared proteins was added to 96-well plate and incubated for 1 hr at room temperature. The plate was washed 3 times with 300 μL of PBS-T, and 100 μL of Ab1 and Ab3 (6.4 pM~500 nM, serial 5-fold dilution, 8 points) diluted with blocking buffer was added to each well and incubated for 1 hr at room temperature. The plate was washed 3 times with 300 μL of PBS-T, and secondary antibody (HRP-conjugated anti-human IgG Fc antibody; Jackson ImmunoResearch, USA) diluted with blocking buffer (1:50,000) was added to each well and incubated for 1 hr at room temperature. The plate was washed 3 times with 300 μL of PBS-T, and 100 μL of TMB-ELISA substrate solution (ThermoFisher Scientific, USA) was added to each well and incubated for 10 min at room temperature. The absorbance (Optical density at nm, OD₄₅₀) was measured at 450 nm using Synergy H1 (BioTek, Winooski, VT). The graphs were generated as sigmoidal dose-response curves and EC₅₀ values were determined by nonlinear regression with a three-parameter logistic equation using GraphPad Prism software (GraphPad Software Inc., San Diego, CA, USA). The Ab1 and Ab3 were dose-dependently bound to human BCAM, and the EC₅₀ value for Ab1 and Ab3 was 1.28 nM and 1.31 nM, respectively. See FIG. 19. and Table 1C.

TABLE 1C
The binding affinity of Ab1 and Ab3 to human BCAM
recombinant protein by sandwich ELISA (EC₅₀ value).

| Antibody | Ab1 | Ab3 |
|---|---|---|
| EC₅₀ (nM) | 1.28 | 1.31 |
| EC₅₀ (μg/mL) | 0.192 | 0.197 |

### Example 1.4 Cellular Binding Affinity of Ab1, Ab2 and Ab3 to HEK293FT Cell Overexpressing Human BCAM by Flow Cytometry

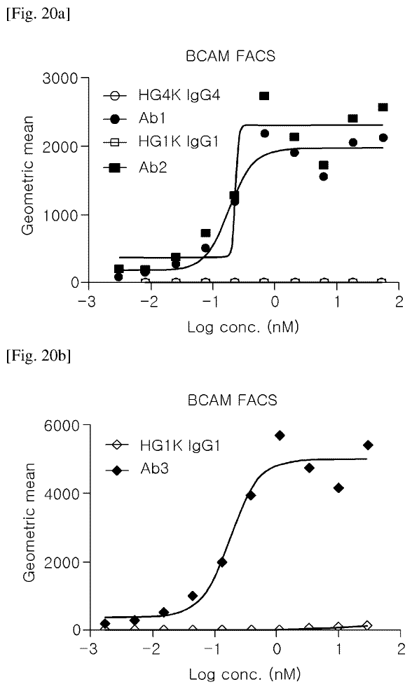

The binding affinity of Ab1, Ab2 and Ab3 to HEK293FT cells overexpressing human BCAM (HEK293FT/hBCAM) was evaluated by flow cytometry analysis. HEK293FT cells (3.0×10⁶) were seeded to 10 cm cell culture dish and incubated for hr at 37° C., 5% CO₂ incubator. The next day, jetPRIME transfection reagent (20 μL) and human BCAM expression plasmid (10 μg) prepared in jetPRIME transfection buffer (200 μL) were mixed and incubated for 10 min at room temperature. The mixture was added to HEK293FT cells drop-wisely and incubated for 4 hr at 37° C., 5% CO₂ incubator. After 4 hr, the mixture from HEK293FT cells was replaced with fresh complete media and incubated for overnight. HEK293FT/hBCAM cells prepared at 1.0×10⁵ cells/100 μL in FACS buffer. The 100 μL of Ab1, Ab2 (3 pM~56 nM, serial 3-fold dilution, 10 points) and Ab3 (1.524 pM~30 nM, serial 3-fold dilution, 10 points), and HG4K IgG4 (SinoBiological, USA) and HG1K IgG1 (SinoBiological, USA) diluted with FACS buffer was added to cells and incubated for 30 min at 4° C. The cells were washed twice with 200 μL of FACS buffer, and then 1 μg/mL of secondary antibody (AlexaFlour® 647 AffiniPure Goat Anti-Human IgG (H+L), JacksonImmunoResearch, USA) diluted with FACS buffer was added to cells and incubated for 30 min at 4° C. in the dark. After washing twice with 200 μL of FACS buffer, and 7-AAD diluted with FACS buffer (1:100) was added to cells and transferred to FACS tubes for analysis. At least 1.0×10⁴ live cells per sample were read using FACSCantoII (BD, USA). The geometric mean fluorescence intensity for assessing the Ab1, Ab2 and Ab3 binding to hBCAM on HEK293FT/hBCAM cells was measured. The graphs were generated as sigmoidal dose-response curves and EC₅₀ values were determined by nonlinear regression with a four-parameter logistic equation using GraphPad Prism software (GraphPad Software Inc., San Diego, CA, USA). The Ab1, Ab2 and Ab3 were dose-dependently bound to HEK293FT/hBCAM cells, and the EC₅₀ value for Ab1, Ab2 and Ab3 was 0.184 nM, 0.232 nM and 0.167 nM, respectively. See FIGS. 20a and 20b and Table 1D and 1E.

TABLE 1D
The binding affinity of Ab1 and Ab2 to HEK293FT
cells overexpressing human BCAM (EC₅₀ value).

| Antibody | Ab1 | Ab2 |
|---|---|---|
| EC₅₀ (nM) | 0.184 | 0.232 |
| EC₅₀ (μg/mL) | 0.027 | 0.034 |

TABLE 1E
The binding affinity of Ab3 to HEK293FT cells
overexpressing human BCAM (EC₅₀ value).

| Antibody | Ab3 |
|---|---|
| EC₅₀ (nM) | 0.167 |
| EC₅₀ (μg/mL) | 0.025 |

### Example 1.5—In Vitro Cellular Internalization Property of Ab1 to MKN-1 Cell Lines Endogenously Expressing BCAM

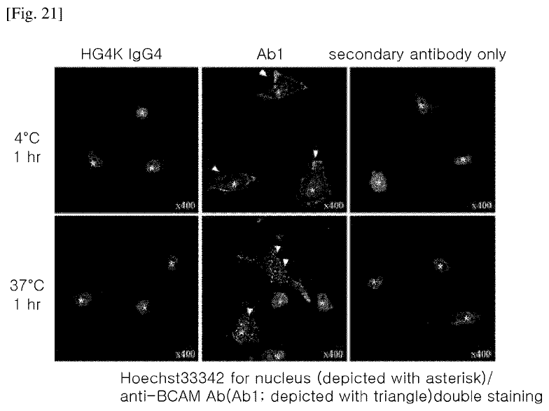

In vitro cellular internalization property by receptor-mediated endocytosis of Ab1 was evaluated to MKN-1 cells endogenously expressing BCAM by immunocytochemical analysis using confocal laser scanning microscopy. MKN-1 (human gastric adenosquamous carcinoma cancer cell line, KCLB) cells were seeded at 5.0×10⁴ cells to each well on the chamber slide (SPL, Korea) and incubated overnight at 37° C. and 5% CO₂ condition. Next day, the chamber slides were washed with 1 mL of PBS and μL of Ab1 (40 μg/mL) or HG4K IgG4 (SinoBiological, USA) (40 μg/mL) diluted in PBS was added to indicated well. The Ab1 treated chamber slides were divided into two groups, one group was used as a negative control for cellular internalization, and the other group was used for induction of cellular internalization. For negative control group, the Ab1 treated cells in chamber slides were incubated for 1 hr at 4° C., and then unbound antibodies were removed and washed 3 times with 1 mL of PBS. The chamber slide was fixed with 500 μL of 4% PFA (paraformaldehyde; Sigma, USA) solution for 10 min at room temperature. While in cellular internalization induction group, the Ab1 treated chamber slides were incubated under conditions of negative control group and then further incubated for 1 hr at 37° C. after washing out unbound antibodies during incubation for 1 hr at 4° C. After unbound antibodies were removed and washed 3 times with 1 mL of PBS, and then chamber slide was fixed with 500 μL of 4% PFA solution for 10 min at room temperature. Fixed chamber slides were washed with 1 mL of PBS twice, and then cells were permeabilized with 1 mL of 0.1% Triton X-100 solution for 15 min at room temperature. After washing with PBS twice, the chamber slides were blocked with 1 mL of 3% BSA blocking solution for 20 min at room temperature. A 500 μL of secondary antibody (10 μg/mL Alexa488-conjugated Goat anti-human IgG; Invitrogen, USA) diluted in blocking buffer was added to each well and the chamber slides were incubated for 1 hr at 4° C. After washing 3 times with PBS, a 500 μL of Hoechst reagent (1:5000) diluted in PBS was added to each well, and the chamber slides were incubated for 3 min at room temperature in the dark. After washing 3 times with PBS, Dako fluorescence mounting solution was drop-wisely added to the slides, and the slides were sealed with clear cover glass. The slides were examined under a confocal laser scanning microscopy (LSM 710 confocal laser scanning microscopy, Carl Zeiss, Germany). For negative control group (incubation for 1 hr at 4° C. without cellular internalization induction, FIG. 21), Ab1 was detected on the cell surface of MKN-1. For cellular internalization induction group (After incubating for 1 hr at 4° C., the cells were transferred to the 37° C. incubator, and then incubated for 1 hr at 37° C.), 40 μg/mL of Ab1 were detected in the intracellular region of MKN-1 (FIG. 21).

### Example 2—Preparation of the Antibody-Drug Conjugates

Antibody-drug conjugates Ab-Linker-DM1, Ab-Linker-MMAF and Ab-Linker-AF were prepared as follows.

### 2.1 List of Abbreviations Used in this Example

Ac: Acetyl
ADC: Antibody-Drug Conjugate
ACN: acetonitrile
AF: Auristatin F
AMAS: N-α-maleimidoacet-oxysuccinimide ester
Cit: Citrulline
CTC: Chlorotrityl chloride
Da: Dalton
DIEA: diisopropylethylamine
DM1: Mertansine
DMF: dimethyl formamide
DMSO: dimethyl sulfoxide
EDC: 1-Ethyl-3-(3-dimethylaminopropyl)carbodiimide
ELSD: Evaporative Light Scattering Detector
Et: ethyl
eq.: equivalent
FA: formic acid
g: gramme
h: hour
HATU: 1-[Bis(dimethylamino)methylene]-1H-1,2,3-triazolo[4,5-b]pyridinium 3-oxide
HOBT: hydroxybenzotriazole
HPLC: high-performance liquid chromatography
HRMS: high resolution mass spectrometry
IgG: Immunoglobulin
IvDde: 1-(4,4-dimethyl-2,6-dioxocyclohex-1-ylidene)isovaleryl
K: kilo
L: litre
Lys: Lysine
m: milli
ma: maleimidoacetic acid
Me: methyl
min: minute
MMAF: Monomethyl auristatin F
mol: molar
MS: mass spectroscopy
m/z: ratio mass over charge
NHS: N-hydroxysuccinimide
nm: nanometer
PBS: phosphate-buffered saline
PEG: polyethylene glycol
PF: CentriPure PF filtration columns
pH: potential for hydrogen
quant.: quantitative
RP: reverse phase
rt: room temperature
Rt: retention time
SEC: size exclusion chromatography
t: tertio
TCEP: tris(2-carboxyethyl)phosphine
TFA: trifluoroacetic acid
Tyr: Tyrosine
UPLC: ultra-performance liquid chromatography
UV: ultraviolet
V: volume
° C.: Celsius degree
m: micro

### 2.2 Starting Materials and Chemicals

The main starting materials and chemicals used in this example are listed below:
Solvents for synthesis and deprotection reagents from Merck or Fischer Scientific AG (Switzerland);
TFA, triethylamine, DIEA, N-hydroxysuccinimide and HOSu from Sigma-Aldrich (Switzerland);
AMAS from Astatech Inc (USA);
Solvents and chemicals for high-performance liquid chromatography (HPLC) and ultra-performance liquid chromatography mass spectrometry (UPLC-MS) from Biosolve (France);
HATU·HPF₆ from Combi-Blocks (USA);
EDC·HCl from Apollo Chemical (USA);
DM1 from Immunogen Inc. (USA);
TCEP·HCl from Fluorochem (UK);
Bromoacetic acid and diisopropylmethanediimine and Pd/C from Acros Organics (Belgium);
H-Cit-Lys(PEG5-ma)-Tyr-OH·TFA (synthesis set out in Section 2.8 below) from Ambiopharm (USA);
PBS (D8537) from Sigma-Aldrich (USA);
AF and MMAF from Angel Pharma Ltd (China).

### 2.3 Methods

The following methods were used to evaluate the compounds of the present disclosure:

### 2.3.1 Purity Determination

The purity of the compounds was determined on UPLC-MS systems:
Method 1: Waters Acquity UPLC System coupled to a Waters SQD mass spectrometer with a CSH C18 column (130 Å, 1.7 μm, 2.1 mm×50 mm) heated at 40° C. using solvent systems A (water+0.1% FA) and B (ACN+0.1% FA) at a flow rate of 0.9 mL/min and a 5-100% gradient of B over 2.7 min.
Method 2: Waters Acquity UPLC System coupled to a Waters SQD mass spectrometer with a CSH Fluoro-phenyl column (130 Å, 1.7 μm, 2.1 mm×50 mm) heated at 40° C. using solvent system A (water+0.1% FA) and B (ACN+0.1% FA) at a flow rate of 0.9 mL/min and a 5-100% gradient of B over 2.9 min.
Method 3: Waters Acquity UPLC System coupled to a Waters SQD mass spectrometer, BEH C18 1.7 μm 50×2.1 mm column heated at 40° C. and fitted with 2 μm insert filter pre-columns (available from Waters), and solvent systems A1 (water+0.1% FA) and B1 (ACN+0.1% FA) at a flow rate of 0.9 mL/min and a 5-100% gradient of B1 over 2.9 min.

### 2.3.2 Aggregates: Size Exclusion Chromatography (SEC)

The aggregate content of the conjugates was determined using the following method and equipment:
Equipment 1 UPLC Waters Acquity
Equipment 2 UPLC Waters Acquity H-Class plus Bio
Detector Tunable UV detector (TUV)
Detector cell Titanium
Pre-column Agilent AdvanceBio SEC 300 Å 2.7 μm 4.6*50 mm
Column Agilent AdvanceBio SEC 300 Å 2.7 μm 4.6*150 mm
Mobile phase Phosphate de potassium 50 mM pH 6.8/250 mM KCl
Wavelength 280 nm
Injection volume 10 μL
Column temperature Ambient
Sample manager temperature 25° C.
Run time 10 min
Flow 0.35 mL/min—Isocratic mode
Weak wash H₂O/ACN (90/10 v/v)
Strong wash H₂O/ACN (10/90 v/v)
Mobile Phase preparation: Potassium phosphate 50 mM pH 6.8/250 mM KCl
Weight 3.61 g KH₂PO₄ and 4.09 g K₂HPO₄ into a 1 L volumetric flask. Complete with milliQ water. If necessary, adjust pH with HCl or NaOH 1 mol/L. Add 18.64 g of KCl.
Sample preparation: Prepare an ADC solution between 1 and 2.5 mg/mL in milliQ water.

### 2.3.3 Concentration: UV Method

Equipment: UV spectrophotometer BioTek Synergy HT
Buffer preparation: PBS pH 7.4: Weight 8.0 g NaCl, 0.2 g KCl, 1.44 g Na₂HPO₄·2H₂O and 0.24 g KH₂PO₄ into a 1 L volumetric flask. Add 900 mL milliQ water. Mix it, when all salts are soluble, adjust pH between 7.35 and 7.44 with HCl 1 mol/L. Complete to 1 L with milliQ water.
Sample preparation: Prepare a 1 mg/mL ADC solution in PBS pH 7.4. Using a UV reader determine the absorbance at 252 and 280 nm.

$${C}^{mAb} = ( \frac{{A}_{280} \times {ε}_{252}^{DM 1} - {A}_{252} \times {ε}_{280}^{DM 1}}{{ε}_{280}^{mAb} \times {ε}_{252}^{DM 1} - {ε}_{252}^{mAb} \times {ε}_{280}^{DM 1}} ) / l \qquad Calculation formula 1$$

$$wherein$$

$$A = absorbance$$

$$DM 1 = relevant antitumor compound , i . e . , DM 1 , MMAF or AF$$

$$L = path length [ cm ]$$

$$C = concentration [ mol / L ]$$

$$ε = extinction coefficient [ L \cdot mol - 1 \cdot cm - 1 ]$$

$$c mg / mL = c mol / L * MW ADC$$

$$MW ADC = MW mAb + ( MW antitumor compound * DAR moyen )$$

$${c}^{ADC} = {Abs}_{280} / ( ( {ε}^{mAb} + DAR \times {ε}^{payload} ) \times L ) \qquad Calculation formula 2$$

$$wherein$$

$$ε = coefficient {d}^{′} extinction [ L \cdot mol - 1 \cdot cm - 1 ] of payload or mAb at 280 nm$$

$$Abs = absorbance at 280 nm$$

$$C = concentration [ mol / L ]$$

$$L = path length [ cm ]$$

$$DAR = Drug - Antibody ratio$$

$$c [ mg / mL ) = c [ mol / L ] * MW ADC$$

$$MW ADC = MW mAb + ( MWpayload * DAR moyen )$$

### 2.3.4 DAR: MS Method

The DAR was measured by native MS of deglycosylated ADC using the following procedure and equipment:
Sample preparation: EndoS reaction: Glycans were cleaved by incubation with EndoS enzyme at acidic pH for 1 h at 37° C. Resulting samples were diluted twice with 50 mM ammonium acetate and 25 μg were injected for each sample.
UPLC: The separation was performed using the MAbPac™ SEC-1 column (Thermo Scientific) and 50 mM ammonium acetate, pH 7, at 0.3 mL/min as mobile phase.
MS: MS was performed using a QExactive HF Orbitrap operated in the high mass range. MS spectra were acquired in the 1800-8000 m/z at a resolution set to 15k, SID eV. The mass spectra were deconvoluted using Protein Deconvolution (Thermo Scientific).

### 2.3.5 Cat B-Induced Cleavage

Cat B-induced cleavage of the antibody-drug conjugates can be evaluated according to the in vitro enzymatic cleavage assay using recombinant human Cathepsin B (commercially acquired from R&D Systems, Bio-Techne AG, cat #. 953-CY-010 in the form of a precursor) and UHPLC-MS/MS analysis.

Briefly, the enzyme is reconstituted in 25 mM 2-(N-morpholino) ethanesulfonic acid (MES) buffer adjusted at pH 5.0 with a 1M NaOH solution and then activated with a nM solution of Dithiothreitol (DDT) at room temperature for at least 15 min. The in vitro enzymatic assay is conducted at 37° C. with the test compound at a concentration of 10 μM (when the test compound is a linker according to the present invention wherein the maleimide group has been quenched in the presence of acetyl-cysteine (Ac-Cys-OH)) or 2.5 μM (when the test compound is an antibody-drug conjugate) in the presence of activated recombinant human Cathepsin B enzyme at 2 μg/mL in a 25 mM MES buffer pH 5.0. The enzymatic cleavage reaction is stopped for each defined time point by mixing a 1/10 ratio volume of acetonitrile+0.1% formic acid (FA) containing an internal standard (warfarine at 0.5 μM). Analysis is conducted using a Waters Acquity UPLC System coupled to a Waters Xevo TQ triple quad mass spectrometer. UHPLC is conducted with a HSS T3 1.8 μm 50×2.1 mm column (Waters) heated at 45° C. and fitted with 2 μm insert filter pre-column (Waters), and solvent systems A1 (H2O+0.1% FA) and B1 (acetonitrile+0.1% FA) at a flow rate of 0.6 mL/min and a 5-95% gradient of B1 over 2.0 min. MS/MS is performed using electrospray ionization (ESI) interface in positive mode and specific MRM transitions for each compound.

### 2.4 Preparation and Characterisation of Linker-Antitumour Compounds

The prepared linker-antitumour compounds are shown in Table 2 below.

TABLE 2
Linker-antitumor compounds

| Linker-antitumor compound | Structure |
|---|---|
| DM1-Linker | 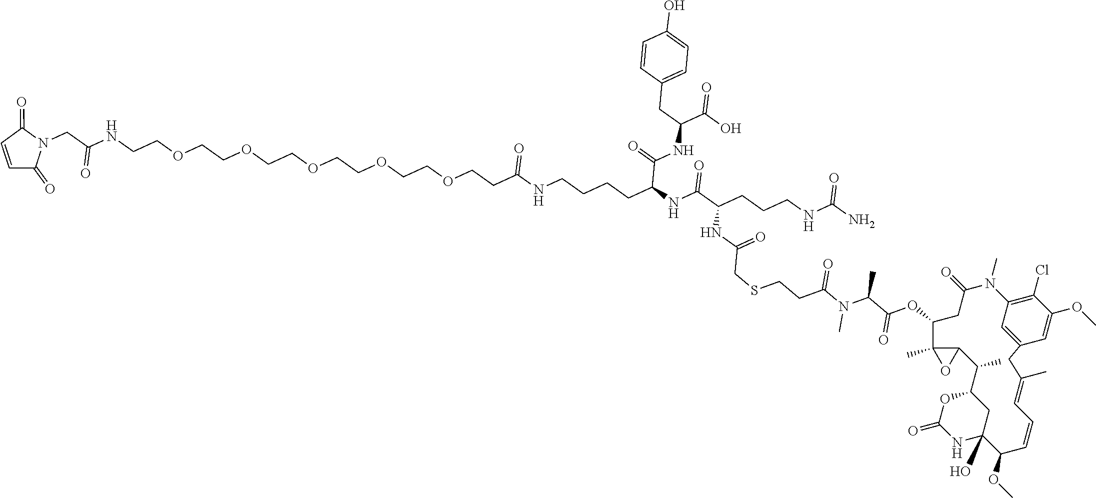 [SMILES: COc1cc2cc(c1Cl)N(C)C(=O)C[C@@H](OC(=O)[C@H](C)N(C)C(=O)CCSCC(=O)N[C@@H](CCCNC(N)=O)C(=O)N[C@@H](CCCCNC(=O)CCOCCOCCOCCOCCOCCNC(=O)CN1C(=O)C=CC1=O)C(=O)N[C@@H](Cc1ccc(O)cc1)C(=O)O)[C@]1(C)OC1[C@H](C)[C@@H]1C[C@@](O)(NC(=O)O1)[C@H](OC)/C=C\C=C(/C)C2] |
| AF Linker | 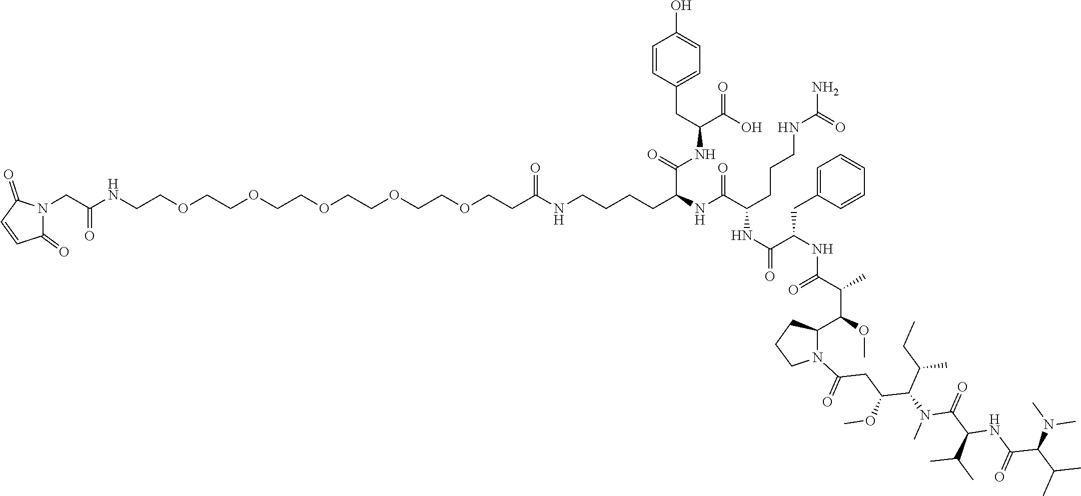 [SMILES: CC[C@H](C)[C@@H]([C@@H](CC(=O)N1CCC[C@H]1[C@H](OC)[C@@H](C)C(=O)N[C@@H](Cc1ccccc1)C(=O)N[C@@H](CCCNC(N)=O)C(=O)N[C@@H](CCCCNC(=O)CCOCCOCCOCCOCCOCCNC(=O)CN1C(=O)C=CC1=O)C(=O)N[C@@H](Cc1ccc(O)cc1)C(=O)O)OC)N(C)C(=O)[C@@H](NC(=O)[C@H](C(C)C)N(C)C)C(C)C] |
| MMAF-Linker | 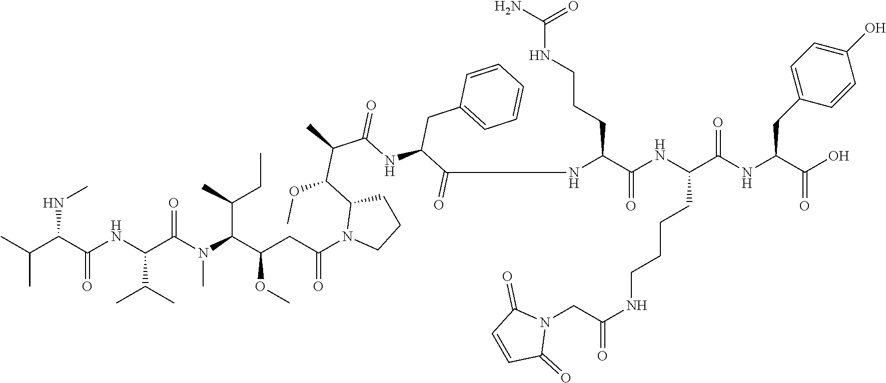 [SMILES: CC[C@H](C)[C@@H]([C@@H](CC(=O)N1CCC[C@H]1[C@H](OC)[C@@H](C)C(=O)N[C@@H](Cc1ccccc1)C(=O)N[C@@H](CCCNC(N)=O)C(=O)N[C@@H](CCCCNC(=O)CN1C(=O)C=CC1=O)C(=O)N[C@@H](Cc1ccc(O)cc1)C(=O)O)OC)N(C)C(=O)[C@@H](NC(=O)[C@@H](NC)C(C)C)C(C)C] |

When purified, the compounds were purified by Preparative Reverse Phase-HPLC on a Buchi C-835 using a Waters column XSelect CSH130 C18 5 μm 19×150 mm OBD with the indicated solvent system at a flow rate of 25 mL/min. The elution was monitored by UV at a wavelength of 220 nm and by ELSD. The purity of the compounds was determined with the UPLC methods described previously (see section 2.3.4).

### 2.5 Preparation of DM1-Ac-Cit-Lys(PEG5-ma)-Tyr (DM1—Linker)

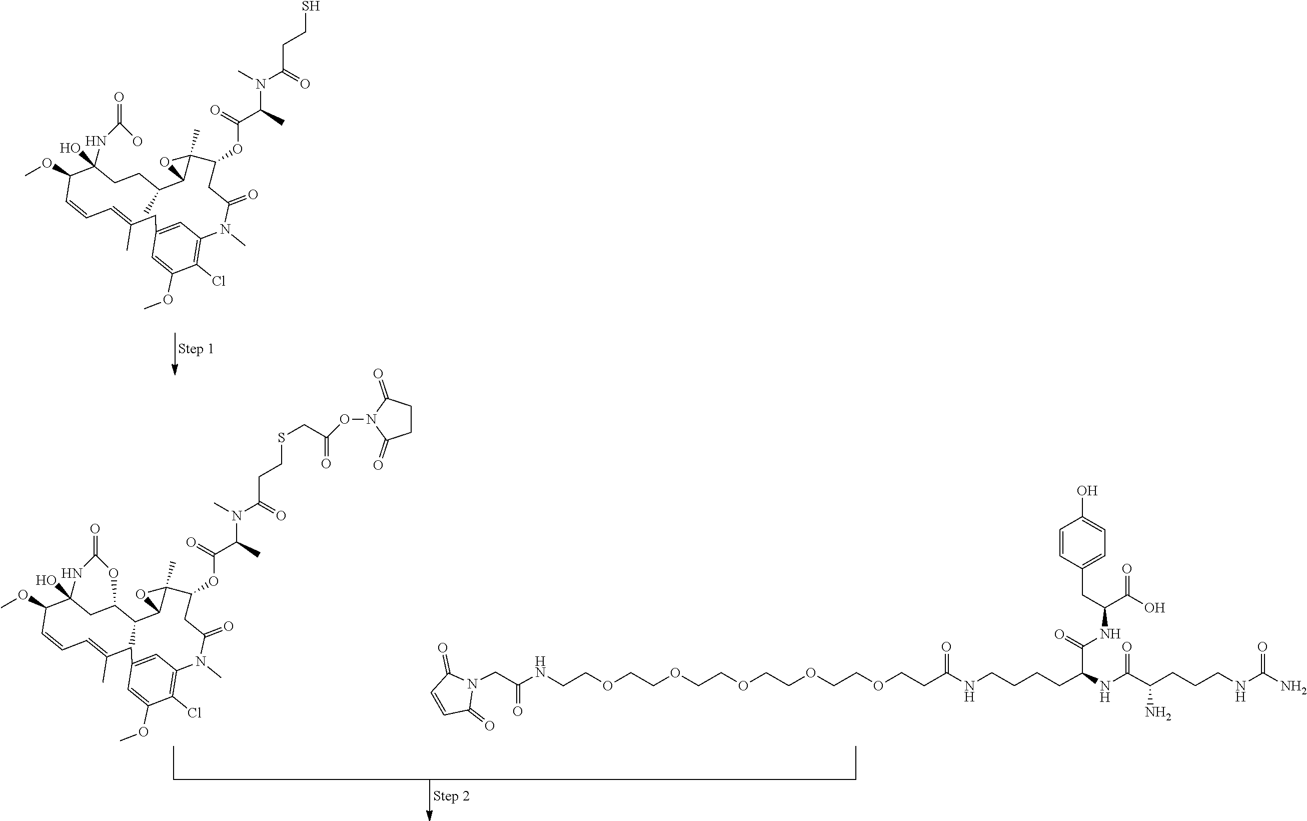

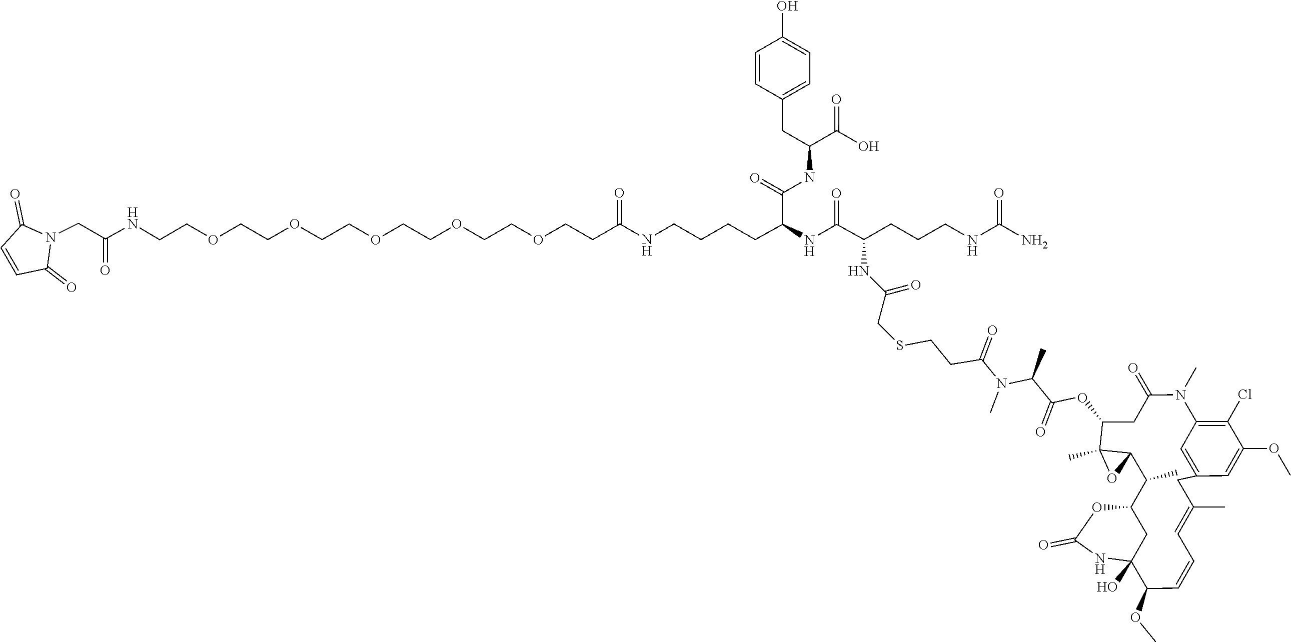

Step 1. DIEA (0.28 mL, 1.63 mmol, 6.0 eq.) was added to a solution of bromoacetic acid (64.8 mg, 0.47 mmol, 1.7 eq.) and DM1 (200 mg, 0.27 mmol, 1.0 eq.) in DMF (2 mL) at rt. After stirring at rt for 1 h, a mixture of HATU·HPF₆ (113.3 mg, 0.30 mmol, 1.1 eq.) and 1-hydroxypyrrolidine-2,5-dione (34.3 mg, 0.30 mmol, 1.1 eq.) was added at rt. After stirring at rt for 30 min TFA was added until acidic pH was reached. Purification on preparative HPLC (30 to 60% ACN+0.1% TFA in water+0.1% TFA) afforded DM1-Ac-NHS ester (134.7 mg, 0.12 mmol, 80% UV purity, 45% yield) as a white powder after freeze-drying. UPLC-MS (method 2): Rt=1.57 min m/z=891 [M−H]⁻.

Step 2. DIEA (31 mL, 0.18 mmol, 4.0 eq.) was added to a mixture of DM1-Ac-NHS (50.0 mg, 44.8 mmol, 1.0 eq.) and H-Cit-Lys(PEG5-ma)-Tyr·TFA (45.2 mg, 44.8 mmol, 1.0 eq.) in DMF (1 mL) at rt. After stirring at rt for 30 min, TFA was added until acidic pH was reached. Purification by preparative HPLC (20 to 50% of ACN+0.1% TFA in water+0.1% TFA) afforded DM1-Ac-Cit-Lys(PEG5-ma)-Tyr (74.9 mg, 44.8 mmol, 100% UV purity, quant.) as a white powder after freeze-drying. UPLC-MS (method 1): Rt=1.31 min, m/z=1672 [M−H]⁻.

### 2.6a Preparation of AF-Cit-Lys(PEG5-ma)-Tyr (AF-Linker)

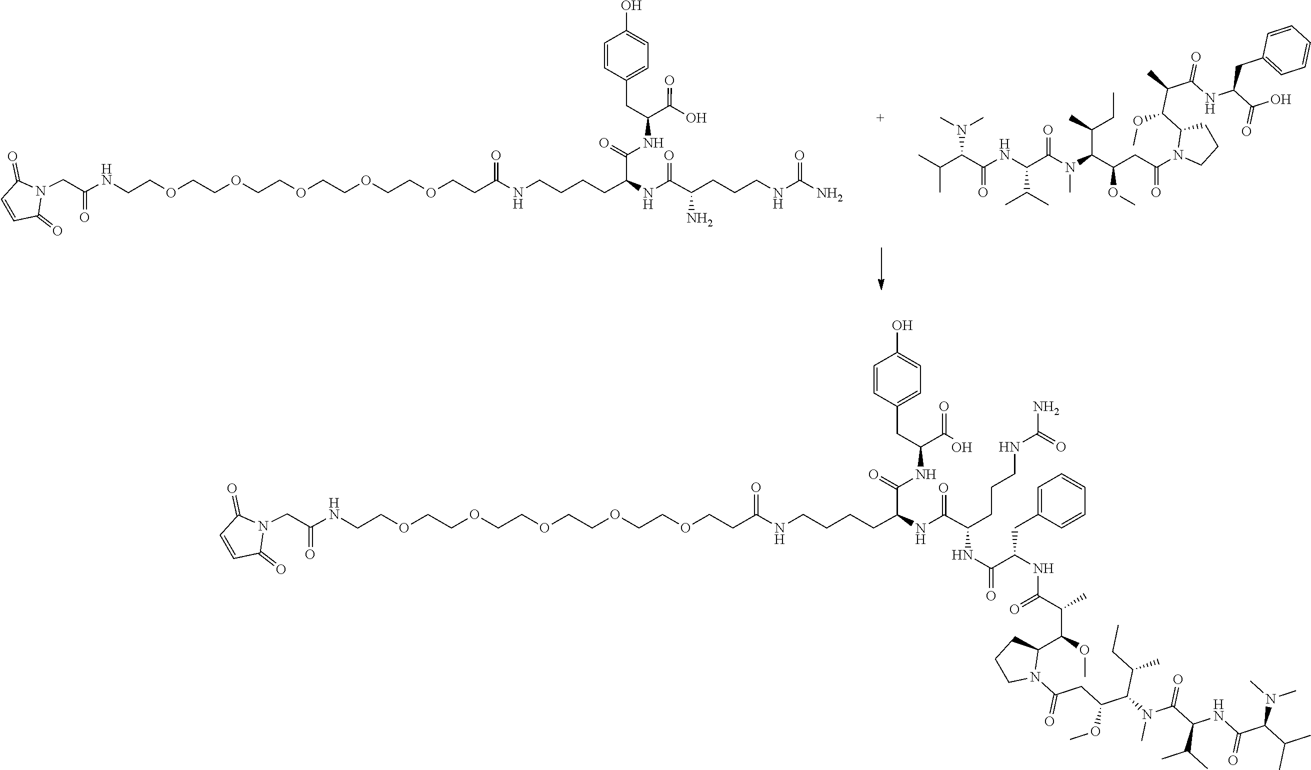

H-Cit-Lys(PEG5-ma)-Tyr·TFA (120.0 mg, 0.12 mmol, 1.0 eq.) was added to a mixture of DIEA (83 mL, 0.48 mmol, 4.0 eq.), HATU·HPF₆ (45.2 mg, 0.12 mmol, 1.0 eq.) and AF·TFA (102.3 mg, 0.12 mmol, 1.0 eq.) in DMF (2.5 mL) at rt. After stirring at rt for 3.5 h, TFA was added until acidic pH was reached. Purification by preparative HPLC (10 to 50% of ACN+0.1% TFA in water+0.1% TFA) afforded AF-Cit-Lys(PEG5-ma)-Tyr (31.7 mg, 19.5 mmol, 100% UV purity, 16% yield) as a white powder after freeze-drying. UPLC-MS (method 3): Rt=1.29 min, m/z=1624 [M+H]⁺, 1622 [M−H]⁻.

### 2.6b Preparation of MMAF-Cit-Lys(ma)-Tyr-OH (MMAF-Linker)

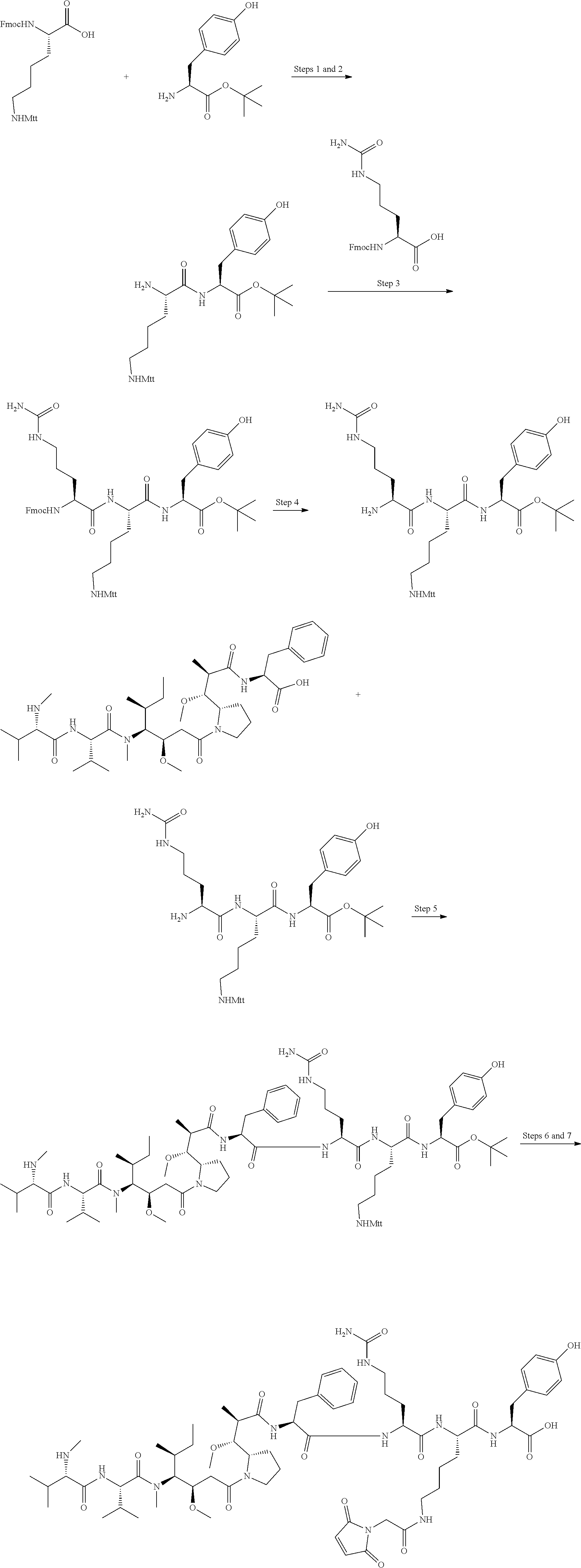

Step 1. DIEA (8.0 mL, 47.1 mmol, 3.0 eq.) was added to a mixture of tert-butyl Ltyrosinate (3.98 g, 16.6 mmol, 1.06 eq.), N2-(((9H-fluoren-9-yl)methoxy)carbonyl)-N6-(diphenyl(p-tolyl)methyl)-L-lysine (10.0 g, 15.7 mmol, 1.0 eq.) and HATU (7.16 g, 18.8 mmol, 1.2 eq.) in DMF (150 mL) at 0° C. After stirring at rt for 30 min, the reaction mixture was poured into EtOAc (500 mL) and washed with ½ sat. brine (2×300 mL). The organic layer was dried over MgSO₄, filtered and concentrated under reduced pressure to afford tert-butyl (2S)-2-[[(2S)-6-[[diphenyl(p-tolyl)methyl]amino]-2-(9H-fluoren-9-ylmethoxycarbonyl amino)hexanoyl]amino]-3-(4-hydroxyphenyl)propanoate (15.46 g, 15.22 mmol, 83% UV purity, 97% yield) as a yellow oil which was used in the next step without further purification. UPLC-MS (method 1): Rt=1.34 min, m/z=844 [M+H]⁺.

Step 2. tert-butyl (2S)-2-[[(2S)-6-[[diphenyl(p-tolyl)methyl]amino]-2-(9H-fluoren-9-ylmethoxycarbonyl amino)hexanoyl]amino]-3-(4-hydroxyphenyl)propanoate (15.46 g, 15.22 mmol, 1.0 eq.) was solubilized in a mixture of DMF/piperidine (70 mL, 9:1 V/V) at rt. After stirring at rt for 25 min, the reaction mixture was concentrated to dryness. Purification by flash chromatography (n-heptane:EtOAc 100:0 to 0:100 then EtOAc:MeOH 95:5 to 90:10) afforded tert-butyl (2S)-2-[[(2S)-2-amino-6-[[diphenyl(p-tolyl)methyl]amino]hexanoyl]amino]-3-(4-hydr oxyphenyl)propanoate (9.80 g, 15.10 mmol, 96% UV purity, 99% yield) as a yellow oil. UPLC-MS (method 2): Rt=1.44 min, m/z=622 [M+H]⁺, 620 [M−H]⁻.

Step 3. To a mixture of tert-butyl (2S)-2-[[(2S)-2-amino-6-[[diphenyl(p-tolyl)methyl]amino]hexanoyl]amino]-3-(4-hydr oxyphenyl)propanoate (6.00 g, 9.25 mmol, 1.0 eq.), (S)-2-((((9H-fluoren-9-yl)methoxy)carbonyl)amino)-5-ureidopentanoic acid (2.94 g, 7.40 mmol, 0.8 eq.) and 3-(((ethylimino)methylene)amino)-N,N-dimethylpropan-1-amine, hydrochloride (2.13 g, 11.1 mmol, 1.2 eq.) in DMF (80 mL) at rt was added 1H-benzo[d][1,2,3]triazol-1-ol hydrate (1.70 g, 11.1 mmol, 1.2 eq.). After stirring at rt for 2 h, the reaction mixture was poured into EtOAc (500 mL) and washed twice with 1/2 sat. brine (400 mL). The organic layer was concentrated under reduced pressure and kept on a high vacuum rotavapor to remove the excess of pyridine. The crude material was purified by flash chromatography (n-heptane:EtOAc 90:10 to plain EtOAc) to afford tert-butyl (2S)-2-[[(2S)-6-[[diphenyl(p-tolyl)methyl]amino]-2-[[(2S)-2-(9H-fluoren-9-ylmethoxy carbonylamino)-5-ureido-pentanoyl]amino]hexanoyl]amino]-3-(4-hydroxyphenyl)propanoate (3.30 g, 3.26 mmol, 99% UV purity, 35% yield) as an off white solid. UPLC-MS (method 2): Rt=1.99 min, m/z=1001 [M+H]⁺.

Step 4. DBU (83 mL, 0.54 mmol, 1.1 eq.) was added to a solution of tert-butyl (2S)-2-[[(2S)-6-[[diphenyl(p-tolyl)methyl]amino]-2-[[(2S)-2-(9H-fluoren-9-ylmethoxy carbonylamino)-5-ureido-pentanoyl]amino]hexanoyl]amino]-3-(4-hydroxyphenyl)propanoate (500 mg, 0.49 mmol, 1.0 eq.) in DMF (3 mL) at rt. After stirring at rt for 20 min, the reaction mixture was added into cold ether (60 mL). The quickly precipitating off-white viscous product was recovered by centrifugation then purified by preparative HPLC (30 to 70% of ACN+0.1% TFA in water+0.1% TFA) to afford tert-butyl (2S)-2-[[(2S)-2-[[(2S)-2-amino-5-ureido-pentanoyl]amino]-6-[[diphenyl(p-tolyl)methyl]amino]hexanoyl]amino]-3-(4-hydroxyphenyl)propanoate; 2,2,2-trifluoroacetic acid (405 mg, 0.45 mmol, 100% UV purity, 92% yield) as a white powder after freeze-drying. UPLC-MS (method 2): Rt=1.35 min, m/z=780 [M+H]⁺, 778 [M−H]⁻.

Step 5. DIEA (0.14 mL, 0.82 mmol, 4.0 eq.) was added to a mixture of tert-butyl (2S)-2—[[(2S)-2-[[(2S)-2-amino-5-ureido-pentanoyl]amino]-6-[[diphenyl(p-tolyl)methyl]amino]hexanoyl]amino]-3-(4-hydroxyphenyl)propanoate;2,2,2-trifluoroacetic acid (201.3 mg, 0.23 mmol, 1.1 eq.), ((2R,3R)-3-((S)-1-((3R,4S,5S)-4-((S)-N,3-dimethyl-2-((S)-3-methyl-2-(methylamino)butanamido)butanamido)-3-methoxy-5-methylheptanoyl)pyrrolidin-2-yl)-3-methoxy-2-methylpropanoyl)-L-phenylalanine (153.1 mg, 0.20 mmol, 1.0 eq.) and 1-[Bis(dimethylamino)methylene]-1H-1,2,3-triazolo[4,5-b]pyridinium 3-oxide hexafluorophosphate (79.9 mg, 0.20 mmol, 1.0 eq.) in DMF (3.5 mL) at rt. After stirring at rt for 40 min, the reaction mixture was added into cold ether (60 mL). Centrifugation afforded tert-butyl (2S)-2-[[(2S)-6-[[diphenyl(p-tolyl)methyl]amino]-2-[[(2S)-2-[[(2S)-2-[[(2R,3R)-3-methoxy-3-[(2S)-1-[(3R,4S,5S)-3-methoxy-5-methyl-4-[methyl-[(2S)-3-methyl-2-[[(2S)-3-methyl-2-(methylamino)butanoyl]amino]butanoyl]amino]heptanoyl]pyrrolidin-2-yl]-2-methyl-propanoyl]amino]-3-phenyl-propanoyl]amino]-5-ureido-pentanoyl]amino]hexanoyl]amino]-3-(4-hydroxyphenyl)propanoate (345 mg, 0.21 mmol, 90% UV purity, quant.) as a yellow oil. UPLC-MS (method 2): Rt=1.66 min, m/z=1494 [M+H]⁺, [M−H]⁻.

Step 6. tert-butyl (2S)-2-[[(2S)-6-[[diphenyl(p-tolyl)methyl]amino]-2-[[(2S)-2-[[(2S)-2-[[(2R,3R)-3-methoxy-3-[(2S)-1-[(3R,4S,5S)-3-methoxy-5-methyl-4-[methyl-[(2S)-3-methyl-2-[[(2S)-3-methyl-2-(methylamino)butanoyl]amino]butanoyl]amino]heptanoyl]pyrrolidin-2-yl]-2-methyl-propanoyl]amino]-3-phenyl-propanoyl]amino]-5-ureido-pentanoyl]amino]hexanoyl]amino]-3-(4-hydroxyphenyl)propanoate (345 mg, 0.21 mmol, 1.0 eq.) was solubilized in a mixture of DCM (4 mL) and TFA (4 mL) at rt. After stirring at rt for 2 h, the reaction mixture was added into cold ether (60 mL). Centrifugation afforded (2S)-2-[[(2S)-6-amino-2-[[(2S)-2-[[(2S)-2-[[(2R,3R)-3-methoxy-3-[(2S)-1-[(3R,4S,5S)-3-methoxy-5-methyl-4-[methyl-[(2S)-3-methyl-2-[[(2S)-3-methyl-2-(methylamino)butanoyl]amino]butanoyl]amino]heptanoyl]pyrrolidin-2-yl]-2-methyl-propanoyl]amino]-3-phenyl-propanoyl]amino]-5-ureido-pentanoyl]amino]hexanoyl]amino]-3-(4-hydroxy phenyl)propanoic acid;2,2,2-trifluoroacetic acid (290 mg, 0.18 mmol, 86% UV purity, 85% yield) as a white solid, which was used in the next step without further purification. UPLC-MS (method 2): Rt=1.07 min, m/z=1181 [M+H]⁺, 1179 [M−H]⁻.

Step 7. DIEA (0.26 mL, 1.59 mmol, 8.0 eq.) was added to a mixture of (2S)-2-[[(2S)-6-amino-2-[[(2S)-2-[[(2S)-2-[[(2R,3R)-3-methoxy-3-[(2S)-1-[(3R,4S,5S)-3-methoxy-5-methyl-4-[methyl-[(2S)-3-methyl-2-[[(2S)-3-methyl-2-(methylamino)butanoyl]amino]butanoyl]amino]heptanoyl]pyrrolidin-2-yl]-2-methyl-propanoyl]amino]-3-phenyl-propanoyl]amino]-5-ureido-pentanoyl]amino]hexanoyl]amino]-3-(4-hydroxy phenyl)propanoic acid;2,2,2-trifluoroacetic acid (280.0 mg, 0.20 mmol, 1.0 eq.) and AMAS (58.1 mg, 0.22 mmol, 1.1 eq.) in DMF (4 mL) at rt. After stirring at rt for 20 min, the reaction mixture was acidified with TFA (80 mL). Purification by preparative HPLC (20 to 70% of ACN+0.1% TFA in water+0.1% TFA) afforded MMAF-Cit-Lys(ma)-Tyr-OH (86.0 mg, 57.3 mmol, 95% UV purity, 29% yield) as a white powder after freeze-drying. UPLC-MS (method 3): Rt=1.03 min, m/z=1430 [M−H]⁻.

### 2.7 Preparation and Characterisation of Antibody-Drug Conjugates (ADCs)

### 2.7.1 Preparation of ADCs

### Preparation of Ab1-Linker-DM1 Conjugate:

A solution of TCEP·HCl (38 μg, 4.0 eq.) in PBS (38 μL) was added to a solution of Ab1 (5 mg, 1.0 eq.) in PBS (0.77 mL) at rt. The reaction mixture was flushed with nitrogen then stirred at 40° C. After stirring at 40° C. for 120 min, a solution of DM1-Ac-Cit-Lys(PEG5-ma)-Tyr (0.56 mg, 10.0 eq.) in DMSO (50 μL) was added. The reaction mixture was stirred at rt for 60 min then diluted to V=2.5 mL with pH8 PBS. Purification using a PF25 column and pH8 PBS as eluent afforded a fraction containing the desired ADC (3.5 mL). The latter fraction was stirred at rt for 16 h then centrifugated (10 min, 4000 rpm) and finally the supernatant was transferred to an Amicon concentrating cell (4 mL, 50 kDa). The mixture was concentrated by centrifugation (12 min, 4500 rpm, 3800 G) to V=0.5 mL. PBS was added (4 mL) and the mixture was concentrated by centrifugation (15 min, 4500 rpm, 3800 G) to V=0.5 mL. PBS was added (4 mL) and the mixture was concentrated by centrifugation (15 min, 4500 rpm, 3800 G) to V=0.5 mL. The final volume was adjusted to V=1.0 mL with PBS. The solution was filtered using a 25 mm PES 0.22 mm Millex filter then aliquoted (2×300 μL, 1×80 μL, 1×50 μL, 1×40 μL) and stored at −80° C. DAR (MS): 3.79. Concentration (UV 280 nm): 3.20 mg/mL or 21.39 mmol/L. Aggregate content: 1.15%. Monomeric content: 98.01%. V=770 μL. m=2.46 mg.

### Preparation of HG4K IgG4-Linker-DM1 Conjugate:

A solution of TCEP·HCl (115 μg, 6.0 eq.) in PBS (115 μL) was added to a solution of HG4K (10 mg, 1.0 eq.) in buffer (1.142 mL) at rt. The reaction mixture was flushed with nitrogen then stirred at 40° C. After stirring at 40° C. for 8 h, a solution of DM1-Ac-Cit-Lys(PEG5-ma)-Tyr (1.56 mg, 14.0 eq.) in DMSO (100 μL) was added. The reaction mixture was stirred at rt for 60 min then diluted to V=2.5 mL with pH8 PBS. Purification using a PF25 column and pH8 PBS as eluent afforded a fraction containing the desired ADC (3.5 mL). The latter fraction was stirred at rt for 16 h then centrifugated (10 min, 4000 rpm) and finally the supernatant was transferred to an Amicon concentrating cell (4 mL, 50 kDa). The mixture was concentrated by centrifugation (10 min, 4500 rpm, 3800 G) to V=0.5 mL. PBS was added (4 mL) and the mixture was concentrated by centrifugation (10 min, 4500 rpm, 3800 G) to V=0.5 mL PBS was added (4 mL) and the mixture was concentrated by centrifugation (10 min, 4500 rpm, 3800 G) to V=0.5 mL. The final volume was adjusted to V=1.0 mL with PBS. The solution was filtered using a 25 mm PES 0.22 mm Millex filter then aliquoted (4×200 μL, 2×50 μL) and stored at −80° C. DAR (MS): 1.74. Concentration (UV 280 nm): 7.00 mg/mL or 48.04 mmol/L. Aggregate content: 2.80%. Monomeric content: 95.3%. V=900 μL. m=6.30 mg.

### Preparation of Ab1-Linker-AF Conjugate:

A solution of TCEP·HCl (0.755 mg, 4.0 eq.) in PBS (755 μL) was added to a solution of Ab1 (98.8 mg, 1.0 eq.) in PBS (19 mL) at rt. The reaction mixture was flushed with nitrogen then stirred at 40° C. After stirring at 40° C. for 120 min, a solution of AF-Cit-Lys(PEG5-ma)-Tyr (10.7 mg, 10.0 eq.) in DMSO (1 mL) was added. The reaction mixture was stirred at rt for 60 min then diluted to V=30 mL with pH8 PBS. Purification using three PF100 column and pH8 PBS as eluent afforded a fraction containing the desired ADC (42 mL). The latter fraction was stirred at rt for 16 h then centrifugated (10 min, 4000 rpm) and finally the supernatant was transferred to an Amicon concentrating cell (15 mL, 50 kDa). The mixture was concentrated by centrifugation (60 min, 4500 rpm, 3800 G) to V=1 mL. PBS was added (14 mL) and the mixture was concentrated by centrifugation (30 min, 4500 rpm, 3800 G) to V=1 mL PBS was added (14 mL) and the mixture was concentrated by centrifugation (30 min, rpm, 3800 G) to V=1 mL. The final volume was adjusted to V=10 mL with PBS. The solution was filtered using a 25 mm PES 0.22 mm Millex filter then aliquoted (18×500 μL, 2×250 μL, 1×150 μL, 2×100 μL, 4×50 μL) and stored at −80° C. DAR (MS): 2.92. Concentration (UV 280 nm): 8.16 mg/mL or 55.17 mmol/L. Aggregate content: 2.48%. Monomeric content: 96.69%. V=10.05 mL. m=82.01 mg.

### Preparation of HG4K IgG4-Linker-AF-Conjugate:

A solution of TCEP·HCl (459 μg, 6.0 eq.) in PBS (459 μL) was added to a solution of HG4K (40 mg, 1.0 eq.) in buffer (4.45 mL) at rt. The reaction mixture was flushed with nitrogen then stirred at 40° C. After stirring at 40° C. for 8 h, a solution of AF-Cit-Lys(PEG5-ma)-Tyr (6.06 mg, 14.0 eq.) in DMSO (400 μL) was added. The reaction mixture was stirred at rt for 70 min then diluted to V=10 mL with pH8 PBS. Purification using a PF100 column and pH8 PBS as eluent afforded a fraction containing the desired ADC (14 mL). The latter fraction was stirred at rt for 16 h then centrifugated (10 min, 4000 rpm) and finally the supernatant was transferred to an Amicon concentrating cell (15 mL, 50 kDa). The mixture was concentrated by centrifugation (20 min, 4500 rpm, 3800 G) to V=0.5 mL. PBS was added (14 mL) and the mixture was concentrated by centrifugation (20 min, 4500 rpm, 3800 G) to V=0.5 mL PBS was added (14 mL) and the mixture was concentrated by centrifugation (20 min, 4500 rpm, 3800 G) to V=0.5 mL. The final volume was adjusted to V=4.5 mL with PBS. The solution was filtered using a 25 mm PES 0.22 mm Millex filter then aliquoted (14×250 μL, 2×200 μL, 4×50 μL) and stored at 80° C. DAR (MS): 1.92. Concentration (UV 280 nm): 6.89 mg/mL or 47.21 mmol/L. Aggregate content: 1.13%. Monomeric content: 95.22%. V=4500 mL. m=31.00 mg.

### Preparation of Ab2-Linker-AF Conjugate:

A solution of TCEP·HCl (103 μg, 2.3 eq.) in PBS (103 μL) was added to a solution of Ab2 (22.38 mg, 1.0 eq.) in PBS (2 mL) at rt. The reaction mixture was flushed with nitrogen then stirred at 40° C. After stirring at 40° C. for 60 min, a solution of AF-Cit-Lys(PEG5-ma)-Tyr (1.87 mg, 7.4 eq.) in DMSO (200 μL) was added. The reaction mixture was stirred at rt for 60 min then diluted to V=5 mL with pH8 PBS. Purification using a PF50 column and pH8 PBS as eluent afforded a fraction containing the desired ADC (7 mL). The latter fraction was stirred at rt for 16 h then centrifugated (10 min, 4000 rpm) and finally the supernatant was transferred to an Amicon concentrating cell (15 mL, 50 kDa). The mixture was concentrated by centrifugation (17 min, 4500 rpm, 3800 G) to V=0.5 mL. PBS was added (14 mL) and the mixture was concentrated by centrifugation (20 min, 4500 rpm, 3800 G) to V=0.5 mL PBS was added (14 mL) and the mixture was concentrated by centrifugation (20 min, 4500 rpm, 3800 G) to V=0.5 mL. The final volume was adjusted to V=2.4 mL with PBS. The solution was filtered using a 25 mm PES 0.22 mm Millex filter then aliquoted (8×250 μL, 1×50 μL, 1×20 μL) and stored at −80° C. DAR (MS): 4.46. Concentration (UV 280 nm): 8.45 mg/mL or 56.08 mmol/L. Aggregate content: 3.92%. Monomeric content: 96.08%. V=2.07 mL. m=17.51 mg.

### Preparation of Ab1-Linker-MMAF Conjugate:

A solution of TCEP·HCl (2.66 mg, 4.5 eq.) in PBS (1.86 mL) was added to a solution of Ab1 (300 mg, 1.0 eq.) in PBS (28.13 mL) at rt. The reaction mixture was purged with nitrogen then stirred at 40° C. After stirring at 40° C. for 4 h15, a solution of MMAF-Cit-Lys(ma)-Tyr-OH (38.66 mg, 25.8 mmol, 12.5 eq.) in DMSO (2.58 mL) was added. The reaction mixture was stirred at rt for 1 h then diluted with 3.6 mL of 10×pH8 PBS. Purification using three PF100 and one PF50 columns and pH8 PBS as eluent, following the manufacturer instructions, afforded the desired ADC (49 mL). The ADC was stirred at rt for 19 h then centrifugated (4 min, 4000 rpm) and finally the supernatant was transferred to six Amicon-15 concentrating cells (15 mL, 50 kDa). The mixture was concentrated by centrifugation (4500 rpm, 3800 G) to V=5 mL, PBS buffer was added (10 mL) and the mixture was concentrated again (4500 rpm, 3800 G) to V=5 mL (this step was repeated twice). The final volume was adjusted to V=30 mL with PBS buffer. The solution was filtered using a 13 mm PES 0.22 μm Millex filter then aliquoted and stored at −80° C. DAR (MS): 3.95. Concentration (UV 280 nm): 9.29 mg/mL or 61.58 mmol/L. Aggregate content: 2.38%. Monomeric content: 97.62%. V=30.48 mL. m=283.01 mg.

### Preparation of Ab3-Linker-MMAF Conjugate:

A solution of TCEP·HCl (0.11 mg, 2.5 eq.) in PBS (77.3 μL) was added to a solution of Ab3 (22.5 mg, 1.0 eq.) in PBS (4.25 mL) at rt. The reaction mixture was purged with nitrogen then stirred at 40° C. After stirring at 40° C. for 1 h30, a solution of MMAF-Cit-Lys(ma)-Tyr-OH (1.85 mg, 8 eq.) in DMSO (247 μL) was added. The reaction mixture was stirred at rt for 1 h then diluted with 0.43 mL of 10×pH8 PBS. Purification using PF50 column and pH8 PBS as eluent, following the manufacturer instructions, afforded the desired ADC (7 mL). The ADC was stirred at rt for 18 h then centrifugated (4 min, 4000 rpm) and finally the supernatant was transferred to Amicon-15 concentrating cell (15 mL, 50 kDa). The mixture was concentrated by centrifugation (4500 rpm, 3800 G) to V=2 mL, PBS buffer was added (13 mL) and the mixture was concentrated again (4500 rpm, 3800 G) to V=2 mL (this step was repeated twice). The final volume was adjusted to V=2 mL with PBS buffer. The solution was filtered using a 13 mm PES 0.22 μm Millex filter then aliquoted and stored at −80° C. DAR (MS): 3.71. Concentration (UV 280 nm): 10.61 mg/mL or 70.44 mmol/L. Aggregate content: 0.65%. Monomeric content: 99.35%. V=1.96 mL. m=20.79 mg.

### Preparation of HG4K IgG4-Linker-MMAF Conjugate:

A solution of TCEP·HCl (3.9 mg, 14 eq.) in PBS (2.7 mL) was added to a solution of HG4K IgG4 (140 mg, 1.0 eq.) in PBS (16.7 mL) at rt. The reaction mixture was purged with nitrogen then stirred at 40° C. After stirring at 40° C. for 5 h, a solution of MMAF-Cit-Lys(ma)-Tyr-OH (4.18 mg, 8 eq.) in DMSO (1.45 mL) was added. The reaction mixture was stirred at rt for 1 h then diluted with 2.32 mL of 10×pH8 PBS. Purification using two PF100, PF25 and PF10 columns and pH8 PBS as eluent, following the manufacturer instructions, afforded the desired ADC (33 mL). The ADC was stirred at rt for 19 h then centrifugated (4 min, 4000 rpm) and finally the supernatant was transferred to three Amicon-15 concentrating cells (15 mL, 50 kDa). The mixture was concentrated by centrifugation (4500 rpm, 3800 G) to V=3 mL, PBS buffer was added (12 mL) and the mixture was concentrated again (4500 rpm, 3800 G) to V=3 mL (this step was repeated twice). The final volume was adjusted to V=14 mL with PBS buffer. The solution was filtered using a 13 mm PES 0.22 μm Millex filter then aliquoted and stored at −80° C. DAR (MS): 4.41. Concentration (UV 280 nm): 9.54 mg/mL or 63.1 mmol/L. Aggregate content: 3.21%. Monomeric content: 96.79%. V=14.21 mL. m=135.54 mg.

### 2.7.2 Summary of Characterisation of Prepared ADCs

All characterisations are given in Table 3 below.

TABLE 3
Characterisations of prepared ADCs

| | Yield | Concentration | | Aggregation |
|---|---|---|---|---|
| ADC number | (%) | (mg/mL) | DAR | (%) |
| HG4K IgG4- | 63 | 7.00 | 1.74 | 2.80 |
| Linker-DM1 | | | | |
| HG4K IgG4- | 78 | 6.89 | 1.92 | 1.13 |
| Linker-AF | | | | |
| Ab1-Linker- | 49 | 3.20 | 3.79 | 1.15 |
| DM1 | | | | |
| Ab1-Linker-AF | 83 | 8.16 | 2.92 | 2.48 |
| Ab2-Linker-AF | 78 | 8.46 | 4.46 | 3.92 |
| Ab1-Linker- | 91 | 9.29 | 3.95 | 2.38 |
| MMAF | | | | |
| Ab3-Linker- | 89 | 10.61 | 3.71 | 0.65 |
| MMAF | | | | |
| HG4K IgG4- | 93 | 9.54 | 4.41 | 3.21 |
| Linker-MMAF | | | | |

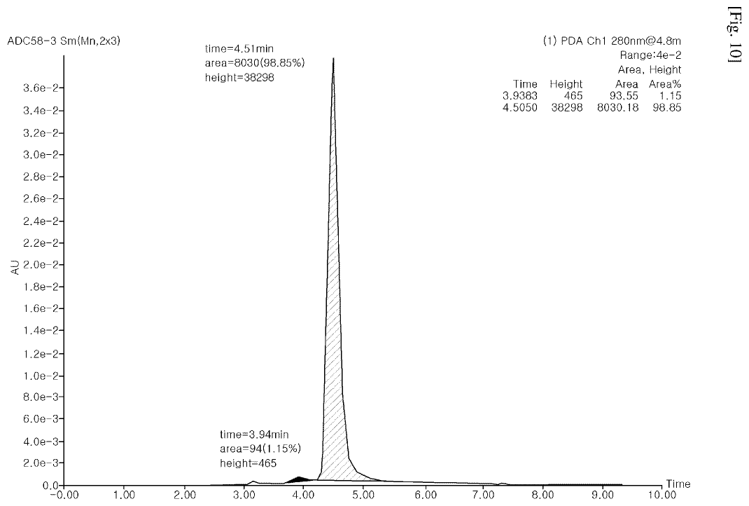

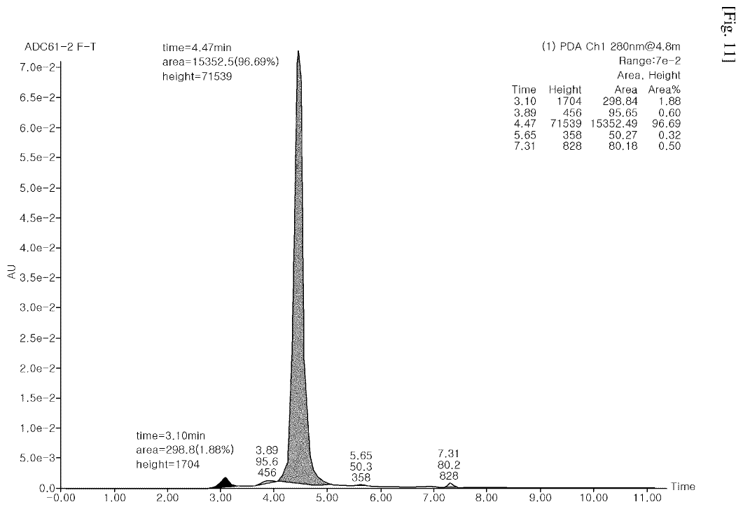

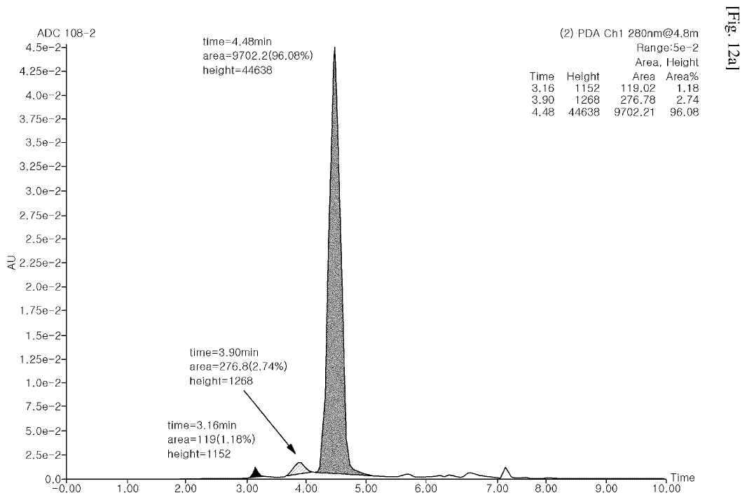

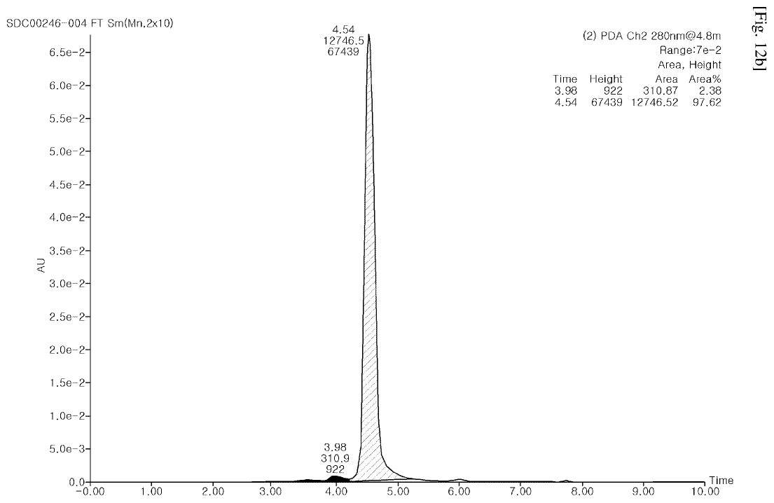

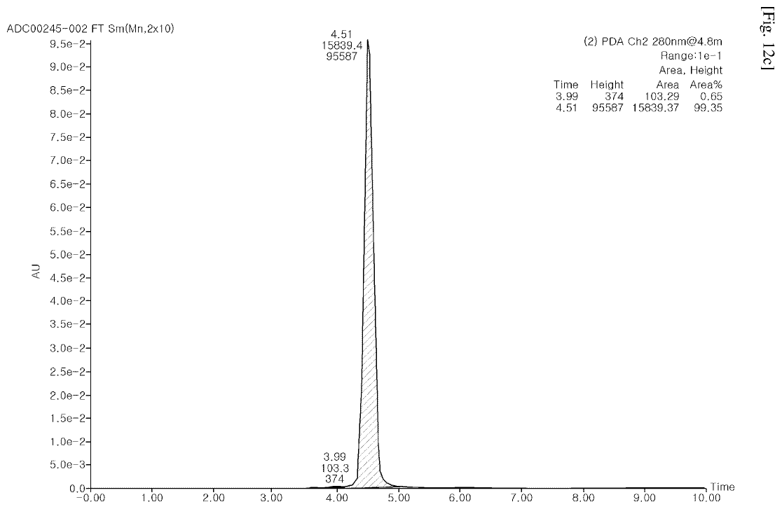

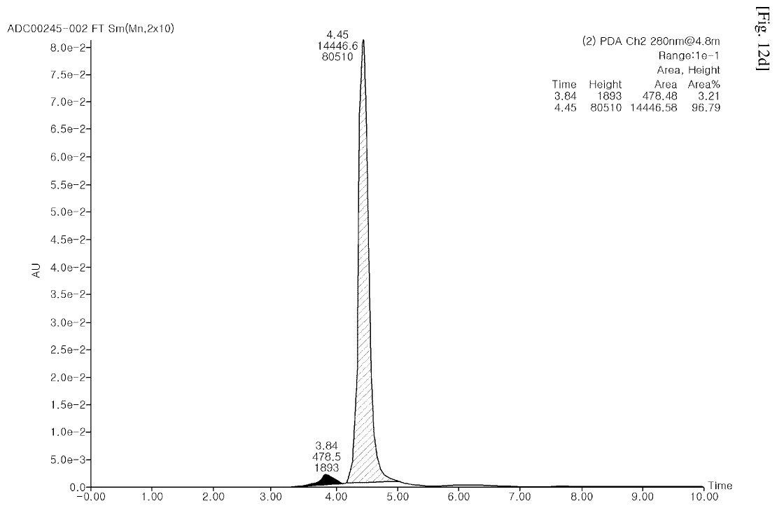

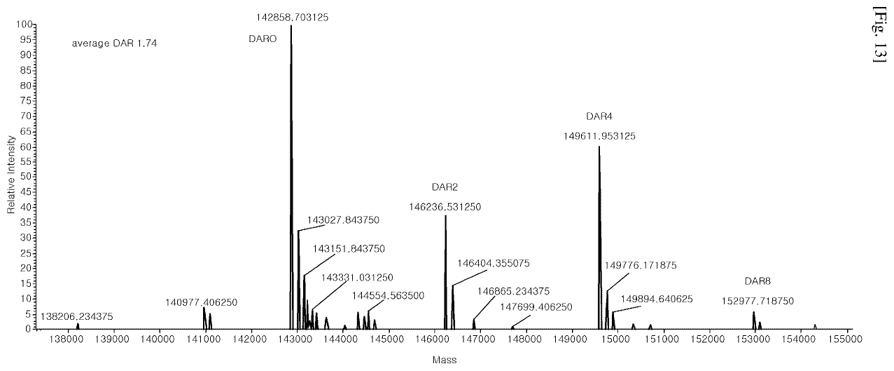

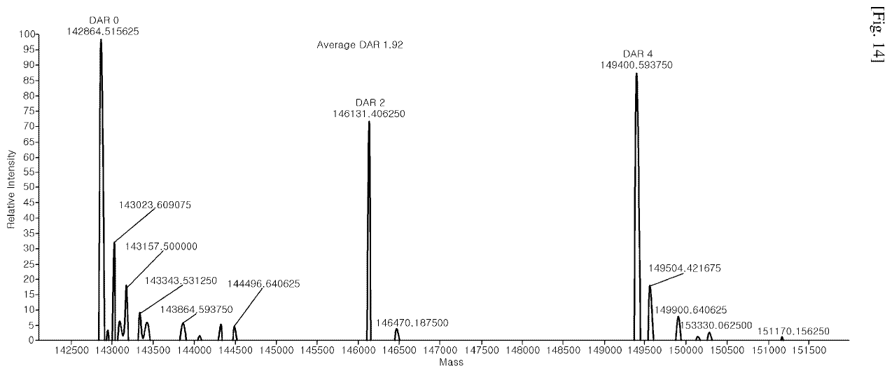

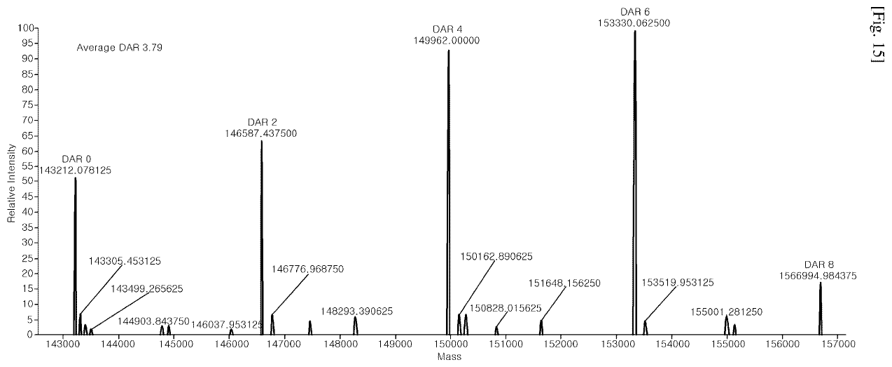

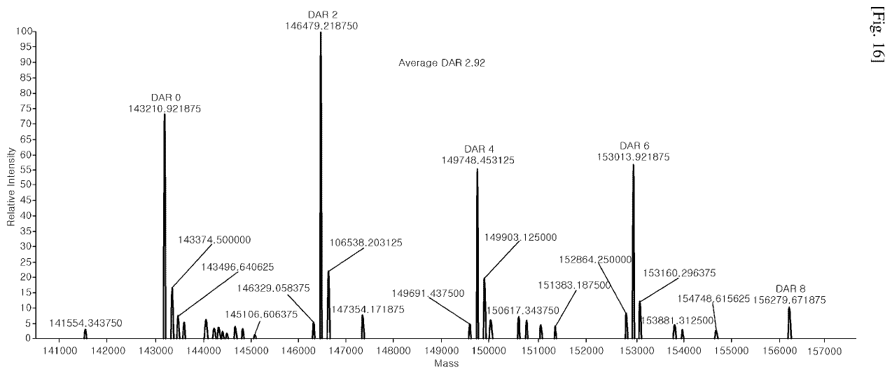

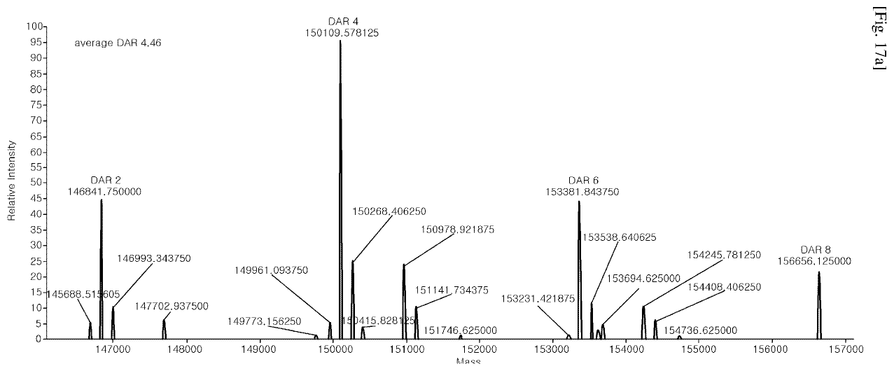

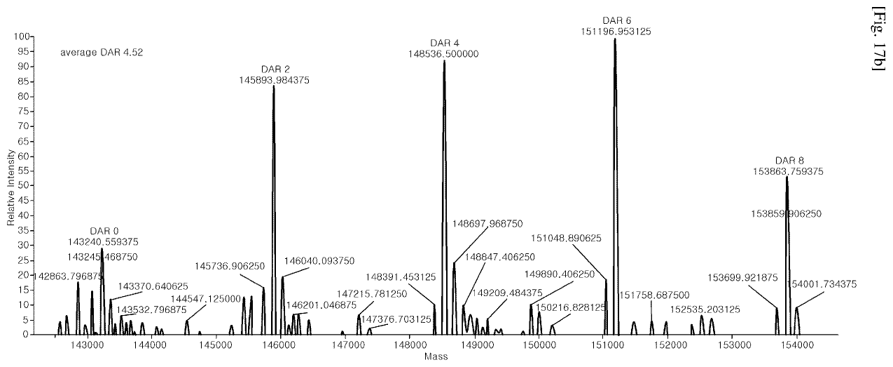

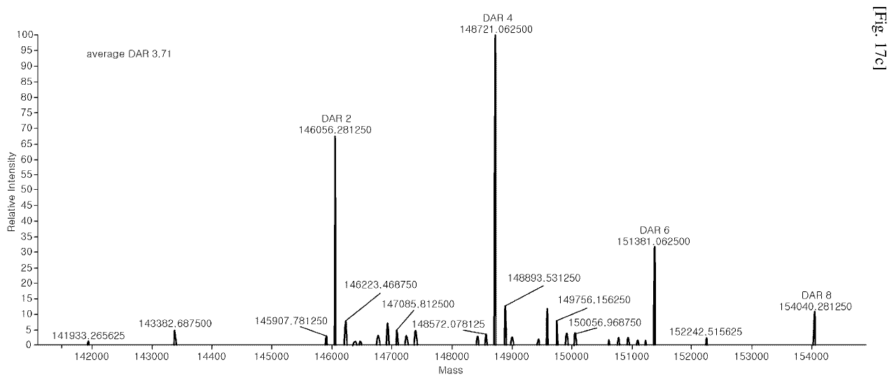

The SEC chromatograms (aggregation content attribution) and mass spectra (MS) of deglycosylated ADCs (DAR attribution) are shown in FIGS. 8 to 17b.

### 2.8 Synthesis of H-Cit-Lys(PEG5-ma)-Tyr-OH·TFA

The steps of the manufacturing process are performed through classical chemical reactions and do not include biochemical processes related to generic engineering or fermentation. All methods used throughout the production of the peptide are based on well documented organic reactions used frequently in peptide chemistry.

All amino acids and related products are purchased from authorized manufacturing or dealers. For each amino acid a manufacturer's certificate of analysis is obtained.

Peptide synthesis is performed on 2-CTC resin according to the general Fmoc-t-butyl (Fmoc/tBu) strategy of solid phase synthesis, with carboxyl group activation carried out by the suitable combination of activating agents, such as diisopropyl carbodiimide/HOBT. Sequentially, each amino acid is coupled to the peptide chain, starting with the C-terminal amino acid. Linear stepwise solid phase peptide synthesis (SPPS) utilizes the principle of total coupling of a temporarily alpha-N-protected amino acid such as Fmoc (9-fluorenyloxycarbonyl) on a derivatized polymeric support. After complete incorporation of the first amino acid, alpha-N-protection of the incorporated residue is removed by orthogonal deblocking solution in order to affect neither the peptide-resin bond nor the side-chain protecting groups. In this way a fully side-chain protected peptide segment anchored to the resin, that contains only one free nucleophilic primary amine on to which the next amino acid is coupled, is obtained. In a stepwise manner, all amino acids are incorporated as active esters according to this procedure. The final amino acid in the sequence is coupled with an N-terminally protected Boc group. The Lys derivative was incorporated with the side chain protected orthogonal to Fmoc such as the ivDde group which is removed with 2% hydrazine in DMF. Following ivDde removal, the sidechain was derivatized with maleimido-PEG5-OH using the activated ester. Subsequently, the peptide is treated with a TFA-based acidolytic cocktail which results in cleavage from the resin and deprotection of the side chain groups, the peptide is then purified by liquid chromatography (RP-HPLC). The purified peptide TFA salt is lyophilized and obtained as a white to off-white powder.

H-Cit-Lys(PEG5-ma)-Tyr-OH·TFA was analyzed for identity and purity by several physicochemical methods: visual inspection, purity by reversed phase HPLC=95% (standard method) and Mass Spectrometry (894.98 Da).

### Example 3

### Example 3.1—Cytotoxic Activity of Ab1-Linker-DM1, Ab1-Linker-AF and Ab2-Linker-AF Against BCAM Expressing Human Cancer Cell Lines

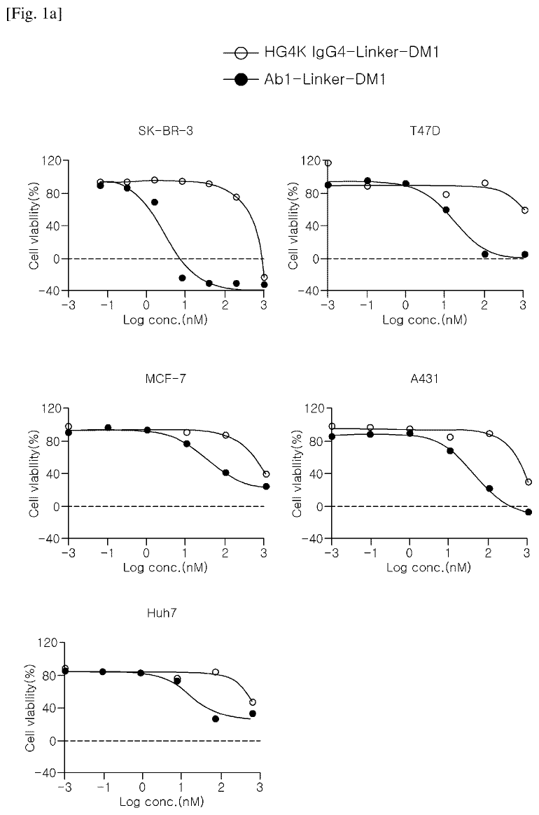

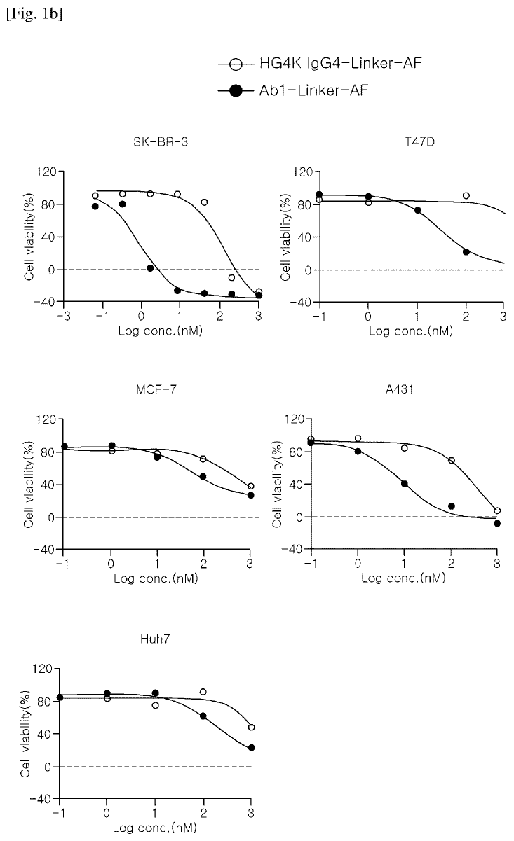

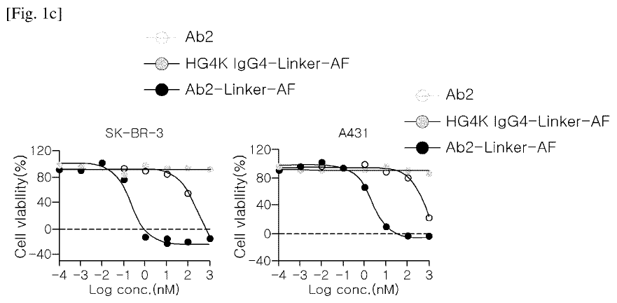

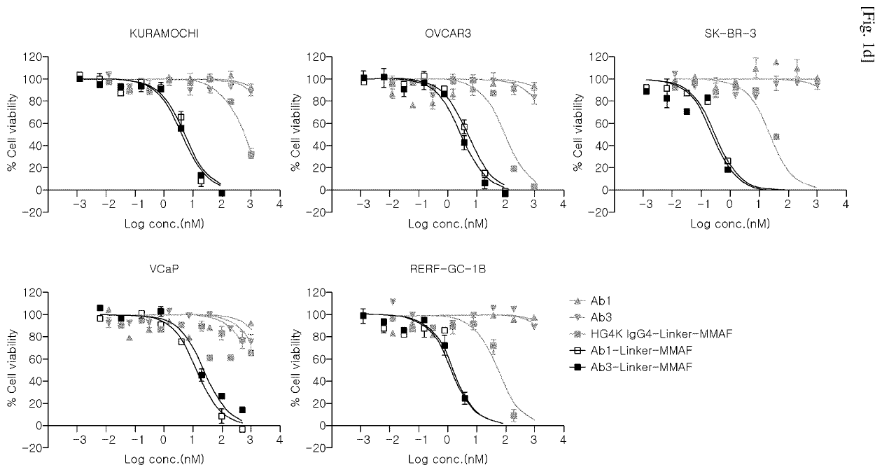

In vitro cytotoxic activity of Ab1-Linker-DM1, Ab1-Linker-AF and Ab2-Linker-AF was assessed in several human cancer cell lines (SK-BR-3, T47D, MCF-7, A431 or Huh7) expressing different levels of BCAM by CellTiter-Glo® assay (Promega). Cells were seeded into 96 well clear flat bottom plates (5,000 cells/well) in RPMI-1640 supplemented with 10% FBS and 1% penicillin/streptomycin and kept at 37° C. and 5% CO 2. After overnight incubation, cells were treated with different concentrations of Ab1-Linker-DM1, Ab1-Linker-AF or negative controls for each test article (HG4K IgG4-Linker-DM1 for Ab1-Linker-DM1 and HG4K IgG4-Linker-AF for Ab1-Linker-AF and Ab2-Linker-AF, respectively). After 72 hours, CellTiter-Glo® reagent was added to each well and luminescence was measured using Synergy H1 (BioTek, Winooski, VT). All experiments were performed in triplicate. The percentage of viable cells was determined using the following formula: (T−D0)/(V−D0)×100 [T=luminescence of drug treated cells, V=luminescence of PBS (vehicle) treated cells, D0=luminescence of untreated cells on day 0]. All data were analyzed using GraphPad Prism software (GraphPad Software, San Diego, CA). As a result, the Ab1-Linker-DM1 and Ab1-Linker-AF significantly inhibited the growth of BCAM high expressing cell lines SK-BR-3, T47D, MCF-7, A431 and Huh7 in a dose dependent manner. See FIGS. 1A (for Ab1-Linker-DM1) and 1B (for Ab1-Linker-AF). In addition, Ab1-Linker-DM1 and Ab1-Linker-AF activities had a strong positive correlation with BCAM expression levels in cancer cell lines. The GI₅₀ values (the dose that inhibits the growth of cells by 50%) for the Ab1-Linker-DM1 against SK-BR3, T47D, MCF-7, A431 and Huh7 cells was 2.8 nM, 16 nM, 34 nM, 42 nM and 22 nM, respectively (Table 4.1). As for Ab1-Linker-AF, the GI₅₀ value in SK-BR-3, T47D, MCF-7, A431 and Huh7 cells was 0.9 nM, 31 nM, 59 nM, 9.1 nM and 193 nM, respectively (Table 4.1). See Table 4.1 below. Next, the cytotoxicity of Ab2-Linker-AF was examined in SK-BR3 and A431 cells. As a result, Ab2-Linker-AF showed a significant cytotoxic activity to tested cells with GI₅₀ of 0.15 nM for SK-BR-3 and 1.7 nM for A431 compared to HG4K IgG4-Linker-AF, while Ab2 itself didn't induce the cytotoxicity of tested cells (FIG. 1C and Table 4.1).

TABLE 4.1
The GI₅₀ value for Ab1-Linker-DM1, Ab1-Linker-AF and
Ab2-Linker-AF in human cancer cell lines in vitro.

| | | | | | |
|---|---|---|---|---|---|
| GI₅₀ (nM) | SK-BR-3 | T47D | MCF-7 | A431 | Huh7 |

Ab1-Linker-DM1

| | | | | | |
|---|---|---|---|---|---|
| Ab1-Linker- | 2.8 | 16 | 34 | 42 | 22 |
| DM1 | | | | | |
| HG4K IgG4- | >1,000 | >1,000 | >1,000 | >1,000 | >1,000 |
| Linker-DM1 | | | | | |

Ab1-Linker-AF

| | | | | | |
|---|---|---|---|---|---|
| Ab1-Linker- | 0.9 | 31 | 59 | 9.1 | 193 |
| AF | | | | | |
| HG4K IgG4- | 134 | >1,000 | 509 | 402 | >1,000 |
| Linker-AF | | | | | |

Ab2-Linker-AF

| GI₅₀ (nM) | SK-BR-3 | A431 |
|---|---|---|
| Ab2-Linker-AF | 0.15 | 1.7 |
| HG4K IgG4- | 85 | 337 |
| Linker-AF | | |
| Ab2 | >1,000 | >1,000 |

### Example 3.2—Cytotoxic Activity of Ab1-Linker-MMAF and Ab3-Linker-MMAF Against BCAM Expressing Human Cancer Cell Lines

In vitro cytotoxic activity of Ab1-Linker-MMAF and Ab3-Linker-MMAF was assessed in several human cancer cell lines (KURAMOCHI, OVCAR3, SK-BR-3, VCaP or RERF-GC-1B) expressing different levels of BCAM by CellTiter-Glo® assay (Promega). Cells were seeded into 96 well clear flat bottom plates (5,000 cells/well) in RPMI-1640 (for KURAMOCHI, OVCAR3, and SK-BR-3) or DMEM (for VCaP and RERF-GC-1B) supplemented with 10% FBS and 1% penicillin/streptomycin and kept at 37° C. and 5% CO₂. After overnight incubation, cells were treated with different concentrations of Ab1-Linker-MMAF, Ab3-Linker-MMAF or negative controls for each test article (HG4K IgG4-Linker-MMAF for Ab1-Linker-MMAF and Ab3-Linker-MMAF, respectively). After 72 hours, CellTiter-Glo® reagent was added to each well and luminescence was measured using Envision 2105 (PerkinElmer, Waltham, MA). All experiments were performed in triplicate. The percentage of viable cells was determined using the following formula: (T−D0)/(V−D0)×100 [T=luminescence of drug treated cells, V=luminescence of PBS (vehicle) treated cells, D0=luminescence of untreated cells on day 0]. All data were analyzed using GraphPad Prism software (GraphPad Software, San Diego, CA). As a result, the Ab1-Linker-MMAF and Ab3-Linker-MMAF significantly inhibited the growth of BCAM high expressing cell lines KURAMOCHI, OVCAR3, SK-BR-3, VCaP and RERF-GC-1B in a dose dependent manner. See FIGS. 1d. Cytotoxic activities of Ab1-Linker-MMAF and Ab3-Linker-MMAF had a strong positive correlation with BCAM expression levels in cancer cell lines. The GI₅₀ values (the dose that inhibits the growth of cells by 50%) for the Ab1-Linker-MMAF against KURAMOCHI, OVCAR3, SK-BR-3, VCaP and RERF-GC-1B cells were 5.3 nM, 5.1 nM, 0.31 nM, 14 nM, and 1.5 nM, respectively (Table 4.2). As for Ab3-Linker-MMAF, the GI₅₀ value in KURAMOCHI, OVCAR3, SK-BR-3, VCaP and RERF-GC-1B cells were 4.4 nM, 3.1 nM, 0.24 nM, 24 nM, and 1.4 nM, respectively (Table 4.2). Ab1-Linker-MMAF and Ab3-Linker-MMAAF showed a significant cytotoxic activity to tested cancer cell lines compared to HG4K IgG4-Linker-MMAF, while Ab1 and Ab3 itself didn't induce the cytotoxicity.

TABLE 4.2
The GI₅₀ value for Ab1-Linker-MMAF and Ab3-Linker-MMAF
in human cancer cell lines in vitro.

| GI₅₀ (nM) | KURAMOCHI | OVCAR3 | SK-BR-3 | VCaP | RERF-GC-1B |
|---|---|---|---|---|---|
| Ab1 | >1,000 | >1,000 | >1,000 | >1,000 | >1,000 |
| Ab3 | >1,000 | >1,000 | >1,000 | >1,000 | >1,000 |
| HG4K IgG4-Linker-MMAF | 580 | 96 | 25 | >500 | 57 |
| Ab1-Linker-MMAF | 5.3 | 5.1 | 0.31 | 14 | 1.5 |
| Ab3-Linker-MMAF | 4.4 | 3.1 | 0.24 | 24 | 1.4 |

### Example 4: In Vivo Anti-Tumor Activity of Ab1-Linker-AF in A431 Xenograft Model

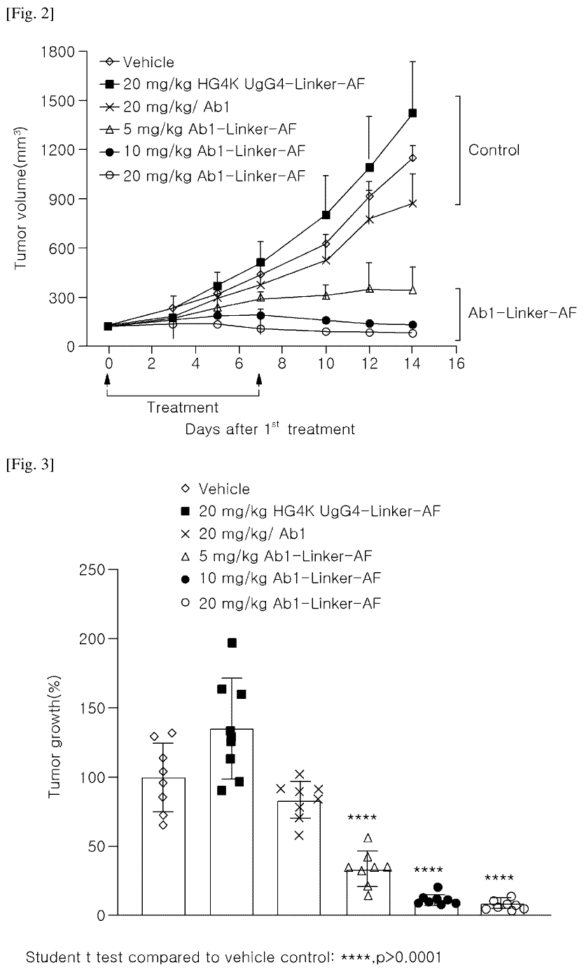

In vivo anti-tumor activity of Ab1-Linker-AF was evaluated in BCAM positive A431 xenograft model. A431 cells (KCLB, Korea) were cultured in RPMI supplemented with 10% FBS and 1% penicillin/streptomycin at 37° C. and 5% CO₂. To establish a tumor model, 5×10⁶ A431 cells were subcutaneously injected into the right flank of a 6-week-old female BALB/c nude mouse (Orientbio, Korea). When tumors reached 75~150 mm³, mice were randomly assigned to the following treatment groups (n=8/group): PBS (vehicle), HG4K IgG4-Linker-AF (20 mg/kg), Ab1 (20 mg/kg), and Ab1-Linker-AF (5, 10, and 20 mg/kg). Each control or test article was given intravenously once a week for two weeks. Tumor diameters were measured 2 to 3 times a week and the tumor volume was calculated with the following formula: [Tumor volume=Length×Width²×0.5]. As a result, Ab1-Linker-AF treatment once weekly for two weeks significantly inhibited the A431 tumor growth in a dose dependent manner. See FIG. 2. On day 14 after starting treatment, the mean tumor growth rate of the vehicle control group was set as 100% and compared with other control groups (20 mg/kg of HG4K IgG4-Linker-AF and 20 mg/kg of Ab1) and Ab1-Linker-AF (5, 10, and 20 mg/kg) treatment groups. The mean tumor growth rate for 5, 10 and 20 mg/kg of Ab1-Linker-AF was 34.2%, 11.4% and 7.3%, respectively (see FIG. 3), while the mean tumor growth rate of HG4K IgG4-Linker-AF and Ab1 was 135.2% and 83.4% each. See FIG. 3. Based on the observation that neither HG4K IgG4-Linker-AF nor Ab1 parental antibody showed potent anti-tumor activity, this study suggests that the anti-tumor activity of Ab1-Linker-AF was the result of the combined effects of Ab1's specific binding to BCAM and the cytotoxic activity of the antitumor compound, AF.

### Reference Example: Cat B-Induced Cleavage Study

The reference compounds shown in Table 5 below were prepared in the Reference Example according to the methodology set out above and in WO 2019/096867 A1.

TABLE 5
Reference compounds comprising a linker of
formula (3)/(4)

| Ref. | |
|---|---|
| Com- | |
| pound | Structure |
| 1 | AF-Arg-Lys(PEG₄-Mal-Cys-Ac)-Phe-OH |
| 2 | AF-Arg-Lys(PEG₄-Mal)-Phe-OH |
| 3 | AF-Arg-Phe-Lys(PEG₄-Mal-Cys-Ac)-OH |
| 4 | AF-Arg-Phe-Lys(PEG₄-Mal)-OH |
| 5 | DM1-Mal-Phe-Lys-Lys(PEG₄-Mal-Cys-Ac)-Phe-OH |
| 6 | DM1-Mal-Phe-Cit-Lys(PEG₄-Mal-Cys-Ac)-Phe-OH |
| 7 | DM1-Mal-Phe-Cit-Phe-Lys(PEG₄-Mal-Cys-Ac)-OH |
| 8 | AF-Cit-Lys(PEG₄-Mal-Cys-Ac)-Phe-OH |
| 9 | ACit-Lys(PEG₄-Mal-Cys-Ac)-Phe-OH |
| 10 | ACit-Phe-Lys(PEG₄-Mal-Cys-Ac)-OH |
| 11 | DM1-Mcc-Phe-Cit-Lys(PEG₅-Mal-Cys-Ac)-Tyr-OH |
| 12 | DM1-Mcc-Cit-Lys(PEG₅-Mal-Cys-Ac)-Tyr-OH |
| 13 | DM1-Mcc-Phe-Lys(PEG₅-Mal-Cys-Ac)-Tyr-OH |

The compounds were prepared by standard Fmoc-based SPPS using an Activo P-li Automated Peptide Synthesizer (available from Activotec), and a Fmoc-Xxx-Wang resin (Xxx: C-terminal amino acid; loading: 0.60 mmol/g; Bachem). Coupling reactions for amide bond formation were performed over 30 min at room temperature using 3 eq of Fmoc-amino-acids, Fmoc-NH-PEG₄-COOH or Fmoc-NH-PEG₅-COOH activated with HBTU (2.9 eq) in the presence of DIEA (7 eq). Fmoc deprotection was conducted with a solution of 20% piperidine in DMF. Selective removal of the Mtt side-chain protecting group (Lys) was performed using DCM/TFA/TJS (94/1/5, v/v/v).

For the synthesis of compounds 1 to 4 and 8, AF was coupled after Fmoc removal by fragment condensation (3 eq AF, 2.9 eq HBTU, 7 eq DIEA) during 30 min. For the synthesis of compounds 9 and 10, Auristatin Cit (ACit) was coupled after Fmoc removal at identical conditions (3 eq ACit, 2.9 eq HBTU, 7 eq DIEA).

For the synthesis of compounds 1 to 4 and 8 to 10, the derivative Mal-PEG₄-NHS was added on resin for 30 min (3 eq of Mal-PEG₄-NHS, 7 eq DIEA) after Mtt removal by DCM/TFA/TJS (94/1/5, v/v/v). Then, for compounds 1, 3, 8, 9 and 10, the maleimide residue on the PEG chain was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS^{a}/water (95/2.5/2.5, v/v/v; ^{a}TIS=triisopropylsilane) during 60 min. After concentration of the cleavage mixture, the crude peptides were precipitated with cold diethyl ether and centrifuged.

For the synthesis of compounds 5 to 7, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. The Mal-derivative was inserted by adding the moiety Mal-NHS to the N-terminus of Phe after Fmoc deprotection. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. Then, Mertansine (DM1, 1.45 eq) was reacted with the terminal maleimide group via chemoselective ligation in PBS buffer at pH 7.4 and acetonitrile (ratio 2:1).

For the synthesis of compounds 11 to 13, the derivative Ma-NHS was added on resin for 30 min (3 eq of Mal-NHS, 7 eq DIEA) after Fmoc removal. Then, the maleimide residue was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. After their purification, the derivative DM1-smcc (1.1 eq) was reacted to the N-terminus of the linker in solution in DMF and 4-methylmorpholine (6 eq) for 4 h.

The peptides were purified on a Waters Autopurification HPLC system coupled to SQD mass spectrometer with a XSelect Peptide CSH C18 OBD Prep column (130 Å, 5 μm, 19 mm×150 mm) using solvent system A (0.1% TFA in water) and B (0.1% TFA in acetonitrile) at a flow rate of 24 mL/min and a 20-60% gradient of B over 30 min.

The appropriate fractions were concentrated and lyophilized. The purity was determined on a Waters Acquity UPLC System coupled to SQD mass spectrometer with a CSH C18 column (130 Å, 1.7 μm, 2.1 mm×50 mm) using solvent system A (0.1% FA in water) and B (0.1% FA in acetonitrile) at a flow rate of 0.6 mL/min and a 5-85% gradient of B over 5 min or CSH Floro-phenyl column (130 Å, 1.7 μm, 2.1 mm×50 mm) using solvent system A (0.1% FA in water) and B (0.1% FA in acetonitrile) at a flow rate of 0.9 mL/min and a 5-95% gradient of B over 2.9 min.

MS-analysis was performed using electrospray ionization (ESI) interface in positive and negative mode. The results of the analysis of the compounds obtained in the

Reference Example are shown in Table 6 below.

TABLE 6
Analysis of compounds 1-13

| Com- | For- | Purity | | [M + | [M + |
|---|---|---|---|---|---|
| pound | mula | (%) | Mw | 2H⁺]⁺² | 3H⁺]⁺³ |
| 1 | C₈₆H₁₃₉N₁₅O₂₃S | 94 | 1783.2 | 892.8 | 595.6 |
| 2 | C₇₉H₁₂₆N₁₄O₁₉ | 96 | 1575.9 | 789.0 | 526.7 |
| 3 | C₈₄H₁₃₅N₁₅O₂₂S | 90 | 1739.1 | 870.8 | 581.0 |
| 4 | C₇₉H₁₂₆N₁₄O₁₉ | 98 | 1575.9 | 789.3 | 526.7 |
| 5 | C₉₅H₁₃₂ClN₁₃O₂₉S₂ | 98 | 2019.7 | 1011.5 | 674.5 |
| 6 | C₉₅H₁₃₁ClN₁₄O₃₀S₂ | 97 | 2048.7 | 1025.6 | 683.8 |
| 7 | C₉₅H₁₃₁ClN₁₄O₃₀S₂ | 94 | 2048.7 | 1026.1 | 685.0 |
| 8 | C₈₄H₁₃₅N₁₅O₂₂S | 90 | 1739.2 | 871.9 | 581.6 |
| 9 | C₇₅H₁₂₅N₁₃O₂₂S | 96 | 1592.9 | 798.3 | 532.2 |
| 10 | C₇₅H₁₂₅N₁₃O₂₂S | 96 | 1592.9 | 798.4 | 532.1 |
| 11 | C₁₀₁H₁₄₁ClN₁₄O₃₂S₂ | 99 | 2162.9 | 1082.2 | 721.9 |
| 12 | C₉₂H₁₃₂ClN₁₃O₃₁S₂ | 89 | 2015.7 | 1008.7 | 673.0 |
| 13 | C₉₅H₁₃₀ClN₁₁O₃₀S₂ | 89 | 2005.7 | — | 669.0 |

The propensity of compounds 1-13 to be cleaved by Cathepsin B was evaluated according to an in vitro enzymatic cleavage assay using recombinant human Cathepsin B and UHPLC-MS/MS analysis as described in Section 2.3.5 above. The results are given in Table 7 below.

TABLE 7
Cat B-induced cleavage study of compounds 1, 3, 5, 6-8,
11 and 12 (Reference: Cys-MC-Val-Cit-PABC-MMAF)

| Ref. | T_{1/2 Compound} | T_{1/2 Reference} | Ratio |
|---|---|---|---|
| Compound | (min) | (min) | (T_{1/2 Compound}/T_{1/2 Reference}) |
| 1 | 1.5 | 12.9 | 0.12 |
| 3 | 1.4 | 12.9 | 0.11 |
| 5 | 0.6 | 10.9 | 0.05 |
| 6 | 0.3 | 13.1 | 0.02 |
| 7 | 1.2 | 13.1 | 0.09 |
| 8 | 1.1 | 12.6 | 0.09 |
| 11 | 0.06 | 11.4 | 0.005 |
| 12 | 0.0004 | 11.4 | 0.00004 |

From these results, it is apparent that exo-Cat B cleavage and drug release (AF-Arg, AF-Cit, ACit, DM1-Mal-Phe-Lys, DM1-Mal-Phe-Cit, DM1-Mcc-Phe-Cit) in compounds 1, 3, 5, 6-8, 11 and 12 occurred simultaneously and were very fast. For instance, Cat B-induced drug release from compound 5 occurred 20 times faster as compared to the reference PABC compound Cys-MC-Val-Cit-PABC-MMAF. The fast cleavage kinetics achieved by compounds 1-8 and 11-12 demonstrates that the compounds of the invention comprising a linker of formula (3)/(4) exhibit high selectivity and binding affinity for the exopeptidase activity of Cat B. Furthermore, it was surprisingly found that the presence of an Ac-Cys-PEG₄ moiety on the side-chain of the Lys residue had no detrimental effect on the binding affinity of the compounds for Cat B. These results also indicate that, by contrast, cleavage by the endopeptidase based mechanism of Cat B as realized in the PABC linker systems (e.g., as in reference compound Cys-MC-Val-Cit-PABC-MMAF) occurs at significantly slower rates. As particular striking examples, compounds 11 and 12 are spontaneously cleaved by exo-Cat B (T_{1/2}<1 min), demonstrating the highly favorable binding properties of substrates based on the linker of formula (3)/(4); it is believed that the favorable interaction between the C-terminal Tyr and occluding loop of Cat B strongly contributes to the fast cleavage rate observed in these compounds.

1. An antibody-drug conjugate of Formula (1): 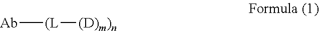
or a pharmaceutically acceptable salt thereof,
wherein Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6,
wherein D is an antitumor compound which is conjugated to the anti-BCAM antibody via the linker,
wherein n is 1 to 10,
wherein m is 1 to 5, and
wherein L is a linker represented by Formula (2): 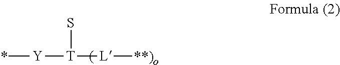
wherein Y is a divalent group comprising one or more atoms selected from C, N, O, P and S,
wherein T is a (1+o)- or (2+o)-valent connecting group,
wherein S is an atom or group that is optionally present to saturate a free valency of T,
wherein L′ is a linker capable of being cleaved by Cathepsin B,
wherein o is an integer of 1 to 5,
wherein * indicates covalent attachment to the anti-BCAM antibody (Ab), and
wherein ** indicates covalent attachment to one or more antitumor compounds (D).

2. The antibody-drug conjugate of claim 1, wherein the heavy chain variable region comprises a sequence of SEQ ID NO: 7, and the light chain variable region comprises a sequence of SEQ ID NO: 8.

3. The antibody-drug conjugate of claim 1, wherein n is 3 to 8 and m is 1.

4. The antibody-drug conjugate of claim 1, wherein a drug (the antitumor compound) to antibody (the anti-BCAM antibody) ratio (DAR) is about to about 8.

5. The antibody-drug conjugate of claim 4, wherein the DAR is about 4.

6. The antibody-drug conjugate of claim 1, wherein the antigen binding fragment thereof is an antibody fragment selected from the group consisting of a Fab fragment, a Fab′ fragment, a Fab′-SH, a Fv fragment, a scFv fragment, a F(ab′)₂ fragment, a VL fragment, a VH fragment, a ScFv-Fc fragment, and a (ScFv)₂-Fc fragment, a diabody, a linear antibody, a fragment produced by a Fab expression library, an anti-idiotypic (anti-Id) antibody, a complementary determining region (CDR), and an epitope-binding fragment.

7. The antibody-drug conjugate of claim 1, wherein the anti-BCAM antibody is a chimeric antibody, a humanized antibody, or a human antibody.

8. The antibody-drug conjugate of claim 1, wherein the linker is covalently attached to the antibody via the side chain of a cysteine comprised in the antibody.

9. The antibody-drug conjugate of claim 1, wherein the linker capable of being cleaved by Cathepsin B (L′) is represented by Formula (3), or Formula (4): 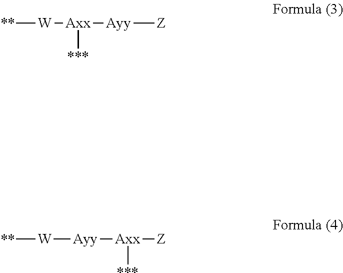
wherein Axx is a trifunctional amino acid, with the proviso that Axx in Formula (3) is not an amino acid in the (D) configuration,
wherein Ayy in Formulae (3) and (4) is an amino acid selected from Phe, Ala, Trp, Tyr, Phenylglycine (Phg), Met, Val, His, Lys, Arg, Citrulline (Cit), 2-amino-butyric acid (Abu), Ornithine (Orn), Ser, Thr, Leu and Ile, or Ayy in Formula (3) is an amino acid selected from homo-tyrosine (homo-Tyr), homo-phenylalanine (homo-Phe), betaphenylalanine (beta-Phe) and beta-homo-phenylalanine (beta-homo-Phe), Tyr(OR₁) and homo-Tyr(OR₁) wherein R₁ is —(CH₂ CH₂O)ₙ₁—R₂, wherein R₂ is a hydrogen atom or a methyl group and n1 is an integer of 2 to 24, with the proviso that Ayy in Formula (4) is not an amino acid in the (D) configuration,
wherein Z is a group covalently attached to the C-terminus of Ayy or Axx selected from —OH and —N(H)(R), wherein R represents a hydrogen atom, an alkyl group or a cycloalkyl group,
wherein W is a drug-carrying unit,
wherein ** indicates covalent attachment to one or more moieties D, and
wherein *** indicates covalent attachment to T,
if more than one linker L′ is present, each linker is independently selected from the aforementioned linkers of Formula (3) and Formula (4).

10. The antibody-drug conjugate of claim 9, wherein at least one, or both of Axx and Ayy is/are defined as follows:
(a) Axx in formula (3) or (4) is an amino acid selected from Glu, 2-amino-pimelic acid (Apa), 2-amino adipic acid (Aaa), 2,3-diamino-propionic acid (Dap), 2,4-diamino-butyric acid (Dab), Lys, Orn, Ser, amino-malonic acid (Ama), and homo-lysine (homo-Lys),
(b) Ayy in Formula (3) is an amino acid selected from Phe, homo-Phe, Ala, Trp, Phg, Leu, Val, Tyr, homo-Tyr, Tyr(OR₁) and homo-Tyr(OR₁), wherein R₁ is —(CH₂CH₂O)ₙ₁—R₂, wherein R₂ is a hydrogen atom or a methyl group and n1 is an integer of 2 to 24, or
(c) Ayy in Formula (4) is an amino acid selected from Phe, homo-Phe, Ala, Trp, Phg, Leu, Val, Tyr and Ser.

11. The antibody-drug conjugate of claim 9, wherein at least one, or both of Axx and Ayy is/are defined as follows:
(a) Axx in formula (3) or (4) is an amino acid selected from Dap, Dab, Lys, Orn and homo-Lys,
(b) Ayy in Formula (3) is an amino acid selected from Phe and Tyr, or
(c) Ayy in Formula (4) is an amino acid selected from Phe and Ser.

12. The antibody-drug conjugate of claim 9, wherein the drug-carrying unit (W) is a group represented by Formula (5): 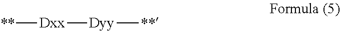
wherein Dxx is absent or an amino acid having a hydrophobic side chain,
wherein Dyy is absent, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein ** indicates covalent attachment to D, and
wherein **′ indicates covalent attachment to the N-terminus of Axx or Ayy.

13. The antibody-drug conjugate of claim 12, wherein at least one, e.g., one or two, of Dxx and Dyy is/are defined as follows:
(a) Dxx is an amino acid selected from Phe, Val, Tyr, homo-Phe and Ala,
(b) Dyy is absent, or an amino acid selected from Arg, Lys, Cit, Orn, Dap and Dab.

14. The antibody-drug conjugate of claim 9, wherein the drug-carrying unit (W) is a group represented by Formula (6), or Formula (7): 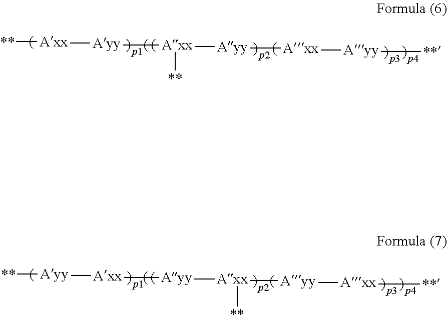
wherein A″xx is a trifunctional amino acid, with the proviso that A″xx in Formula (6) is not an amino acid in the (D) configuration,
wherein A′yy is an amino acid selected from Phe, Ala, Trp, Tyr, Phg, Met, Val, His, Lys, Arg, Cit, Abu and Orn, with the proviso that A′yy in Formula (7) is not an amino acid in the (D) configuration; if more than one A′yy are present, each A′yy is independently selected from the aforementioned amino acids,
wherein A″yy is an amino acid selected from Phe, Ala, Trp, Tyr, Phg, Met, Val, His, Lys, Arg, Cit, Abu and Orn, with the proviso that A″yy in Formula (7) is not an amino acid in the (D) configuration; if more than one A″yy are present, each A″yy is independently selected from the aforementioned amino acids,
wherein A′″yy is an amino acid selected from Phe, Ala, Trp, Tyr, Phg, Met, Val, His, Lys, Arg, Cit, Abu and Orn, with the proviso that A′″yy in Formula (7) is not an amino acid in the (D) configuration; if more than one A′″yy are present, each A′″yy is independently selected from the aforementioned amino acids,
wherein A′xx is an amino acid, with the proviso that A′xx in Formula (6) is not an amino acid in the (D) configuration,
wherein A′″xx is an amino acid, with the proviso that A′″xx in Formula (6) is not an amino acid in the (D) configuration,
wherein p1 is an integer of 0 to 3,
wherein p2 is 0 or 1,
wherein p3 is an integer of 0 to 3, with the proviso that if p2 is 0, p3 is not 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.

15. The antibody-drug conjugate of claim 14, wherein at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Lys, homo-Lys, Cit, Orn, Dap and Dab,
(c) A′″xx is an amino acid selected from Arg, Lys, homo-Lys, Cit, Orn, Dap and Dab,
(d) A′yy is an amino acid selected from Phe, Ala, Trp, Phg and Tyr,
(e) A″yy is an amino acid selected from Phe, Ala, Trp, Phg and Tyr,
(f) A′″yy is an amino acid selected from Phe, Ala, Trp, Phg and Tyr.

16. The antibody-drug conjugate of claim 9, wherein the drug-carrying unit is a group represented by Formula (8): 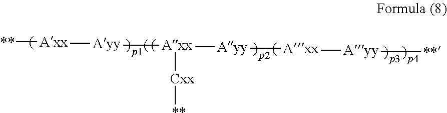
wherein A″xx is a trifunctional amino acid selected from Glu, α-amino adipic acid (Aaa), Dap, Ser, Thr, homo-serine (homo-Ser), homo-threonine (homo-Thr) and amino malonic acid (Ama), with the proviso that A″xx is not an amino acid in the (D) configuration,
wherein Cxx is a single covalent bond unless A″xx is Ama; if A″xx is Ama, Cxx is Pro or an N-methyl amino acid, the N-terminus of Cxx binds to a carboxyl end of Ama and the C-terminus of Cxx is covalently attached to one moiety D,
wherein A′yy, A″yy and A′″yy are each independently an amino acid selected from Phe, Ala, Trp, Tyr, Phg, Met, Val, His, Lys, Arg, Cit, Abu, Orn,
wherein A′xx and A′″xx are each independently an amino acid, with the proviso that A′xx and A′″xx are not an amino acid in the (D) configuration,
wherein p1 is 0 or 1,
wherein p2 is 0 or 1,
wherein p3 is an integer of 0 to 3, with the proviso that if p2 is 0, p3 is not 0,
wherein p4 is an integer of 1 to 4, with the proviso that p4 and o in Formula (2) are selected such that m in Formula (1) is an integer of 1 to 5,
wherein * indicates covalent attachment to the N-terminus of Axx or Ayy, and
wherein ** indicates covalent attachment to an antitumor compound.

17. The antibody-drug conjugate of claim 16, wherein at least one, e.g., one, two, three, four, five or six, of A′xx, A″xx, A′″xx, A′yy, A″yy and A′″yy is/are defined as follows:
(a) A′xx is an amino acid selected from Arg, Lys, homo-Lys, Cit, Orn, Dap and Dab,
(b) A″xx is an amino acid selected from Dap, Dab, Lys, Orn and homo-Lys,
(c) A′″xx is an amino acid selected from Arg, Lys, homo-Lys, Cit, Orn, Dap and Dab,
(d) A′yy is an amino acid selected from Phe, Ala, Trp, Phg and Tyr,
(e) A″yy is an amino acid selected from Phe, Ala, Trp, Phg and Tyr,
(f) A′″yy is an amino acid selected from Phe, Ala, Trp, Phg and Tyr.

18. The antibody-drug conjugate of claim 1, wherein the connecting group (T) is represented by Formula (9): 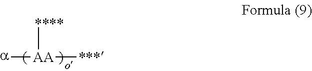
wherein each AA is independently a moiety comprising a trifunctional amino acid,
wherein α indicates covalent attachment of the N-terminus of AA, or the N-terminus of the first AA in case of o′ being 2 to 5, to Y,
wherein o′ is an integer of 1 to 5, with the proviso that o′ is 1 to 4 if another moiety L′ is attached to ***′,
if o′ is 1, the side chain of the trifunctional amino acid is covalently attached to S or L′, the C-terminus being covalently attached to the other moiety L′ or S, respectively,
if o′ is 2, 3, 4 or 5, **** indicates covalent attachment to L′, and ***′ indicates covalent attachment to S.

19. The antibody-drug conjugate of claim 18, wherein each AA is independently a moiety comprising an amino acid selected from NF-propargyloxycarbonyl-L-Lysine (Lys(Poc)), Asp, Glu, Orn, Lys, Dab and Dap.

20. The antibody-drug conjugate of claim 1, wherein the connecting group (T) is represented by Formula (10), or Formula (11): 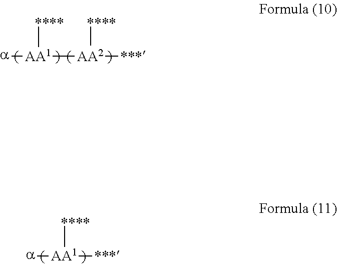
wherein each AA¹ and AA² is independently a moiety comprising a trifunctional amino acid,
wherein α indicates covalent attachment to Y,
wherein, in Formula (11), the side chain of the trifunctional amino acid is covalently attached to L′ or S, the C-terminus is covalently attached to the other moiety S or L′, respectively,
wherein, in Formula (10), **** indicates covalent attachment to L′, and ***′ indicates covalent attachment to S or L′.

21. The antibody-drug conjugate of claim 20, wherein each AA¹ and AA² is independently a moiety comprising an amino acid selected from Lys(Poc), Asp, Glu, Orn, Lys, Dab and Dap.

22. The antibody-drug conjugate of claim 1, wherein the connecting group (T) is represented by Formula (12), or Formula (13): 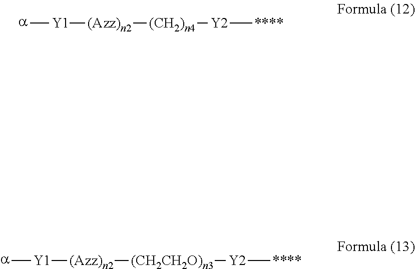
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to L′.

23. An antibody-drug conjugate of Formula (1): 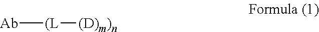
or a pharmaceutically acceptable salt thereof,
wherein Ab is an anti-basal cell adhesion molecule (BCAM) antibody or an antigen binding fragment thereof, comprising (i) a heavy chain variable region comprising a VH CDR1 sequence of SEQ ID NO: 1, a VH CDR2 sequence of SEQ ID NO: 2, and a VH CDR3 sequence of SEQ ID NO: 3, and (ii) a light chain variable region comprising a VL CDR1 sequence of SEQ ID NO: 4, a VL CDR2 sequence of SEQ ID NO: 5, and a VL CDR3 sequence of SEQ ID NO: 6,
wherein D is an antitumor compound which is conjugated to the anti-BCAM antibody via the linker,
wherein n is 1 to 10,
wherein m is 1 to 5, and
wherein the linker (L) is represented by Formula (14), or Formula (15): 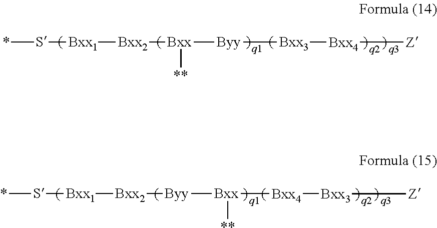
wherein Bxx in Formulae (14) and (15) is a trifunctional amino acid, with the proviso that Bxx in Formula (14) is not in the (D) configuration,
wherein Byy in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂,
wherein R₂ is a hydrogen atom or a methyl group and n1 is an integer of 2 to 24, or Byy in Formula (14) is an amino acid selected from homo-Tyr, homo-Tyr(OR₁) wherein R₁ is as defined above, homo-Phe, beta-Phe and beta-homo-Phe; with the proviso that if q1*q3>1 and q2=0, only the C-terminal Byy in Formula (14) may be an amino acid selected from beta-Phe and beta-homo-Phe; with the proviso that Byy in Formula (15) is not in the (D) configuration,
wherein Bxx₁ in Formulae (14) and (15) is a single covalent bond or an amino acid having a hydrophobic or basic side chain,
wherein Bxx₂ in Formulae (14) and (15) is an amino acid having a hydrophobic or basic side chain,
wherein Bxx₃ in Formulae (14) and (15) is an amino acid, with the proviso that Bxx3 in Formula (14) is not in the (D) configuration,
wherein Bxx₄ in Formulae (14) and (15) is an amino acid selected from Phe, homo-Phe, Ala, Trp, Tyr, Phg, Val, His, Lys, Abu, Met, Cit, Orn, Ser, Thr, Leu, Ile, Arg and Tyr(OR₁), wherein R₁ is —(CH₂CH₂O)ₙ₁—R₂,
wherein R₂ is a hydrogen atom or a methyl group and n1 is an integer of 2 to 24, or Bxx4 in Formula (14) is an amino acid selected from homo-Tyr, homo-Tyr(OR₁), homo-Phe, beta-Phe and beta-homo-Phe;
with the proviso that if q2*q3>1, only the C-terminal Bxx4 in Formula (14) may be an amino acid selected from beta-Phe and beta-homo-Phe; with the proviso that Byy in Formula (15) is not in the (D) configuration,
or wherein the linker (L) is represented by Formula (16): 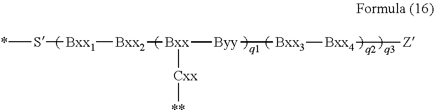
wherein Bxx in Formula (16) is a carboxylic amino acid, or a trifunctional amino acid selected from Dap, Dab, Ser, Thr, Lys, Orn, homo-Lys, homo-Ser and homo-Thr, with the proviso that Bxx is not in the (D) configuration,
wherein Cxx is a single covalent bond unless Bxx is Ama, if Bxx is Ama, Cxx is Pro or an N-methyl amino acid, the N-terminus of Cxx binds to a carboxyl group of Ama and the C-terminus of Cxx is covalently attached to one moiety D,
wherein Byy in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂,
wherein R₂ is a hydrogen atom or a methyl group and n1 is an integer of 2 to 24; with the proviso that if q1*q3>1 and q2=0, only the C-terminal Byy may be an amino acid selected from beta-Phe and beta-homo-Phe,
wherein Bxx₁ in Formula (16) is a single covalent bond or an amino acid having a hydrophobic or basic side chain,
wherein Bxx₂ in Formula (16) is an amino acid having a hydrophobic or basic side chain,
wherein Bxx₃ in Formula (16) is an amino acid, with the proviso that Bxx3 is not in the (D) configuration,
wherein Bxx₄ in Formula (16) is an amino acid selected from Phe, homo-Phe, Ala, Trp, Tyr, Phg, Val, His, Lys, Abu, Met, Cit, Orn, Ser, Thr, Leu, Ile, Arg, homo-Phe, beta-Phe, beta-homo-Phe, homo-Tyr, Tyr(OR₁) and homo-Tyr(OR₁), wherein R₁ is —(CH₂CH₂O)ₙ₁—R₂,
wherein R₂ is a hydrogen atom or a methyl group and n1 is an integer of 2 to 24; with the proviso that if q2*q3>1, only the C-terminal Bxx4 may be an amino acid selected from beta-Phe and beta-homo-Phe,
wherein S′ is a divalent group comprising one or more atoms selected from C, N, O, P and S,
wherein Z′ is a group covalently attached to the C-terminus of Byy or Bxx4 in Formulae (14) and (16) or the C-terminus of Bxx or Bxx3 in Formula (15), which is selected from —OH and —N(H)(R), wherein R represents a hydrogen atom, an alkyl group or a cycloalkyl group,
wherein q1 is an integer of 0 to 5,
wherein q2 is an integer of 0 to 3, with the proviso that if q1 is 0, q2 is not 0,
wherein q3 is an integer of 1 to 5,
wherein q1, q2 and q3 are selected such that m in Formula (1) is an integer of 1 to 5,
wherein * indicates covalent attachment to the anti-BCAM antibody (Ab), and
wherein each ** indicates covalent attachment to one moiety D.

24. The antibody-drug conjugate of claim 23, wherein at least one, e.g., one, two, three, four, five or six, of Bxx, Byy, Bxx₁, Bxx₂, Bxx₃ and Bxx₄ is/are defined as follows:
(a) Bxx is an amino acid selected from Dap, Dab, Lys, Orn, Ser, Glu, Ama, Thr, Tyr, Aaa, homo-Ser and homo-Thr,
(b) Byy is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24,
(c) Bxx₁ is a single covalent bond, or an amino acid selected from Phe, homo-Phe, Phg, Val, Ser, Tyr, Ala, Leu and Ile,
(d) Bxx₂ is an amino acid selected from Arg, Lys, Cit, Val, Leu, Ser, Ala, Gly, His, Gln, Phg and Phe,
(e) Bxx₃ is an amino acid selected from Phe, homo-Phe, Phg, Val, Ser, Tyr, Ala, Leu and Ile,
(f) Bxx₄ is an amino acid selected from Cit, Phe, homo-Phe, Ser, Trp, Tyr and Tyr(OR₁), wherein R₁ is —(CH₂CH₂O)ₙ₁—R₂, wherein R₂ is a hydrogen atom or a methyl group and n1 is an integer of 2 to 24.

25. The antibody-drug-conjugate of claim 1, which is represented by Formula (17), or Formula (18): 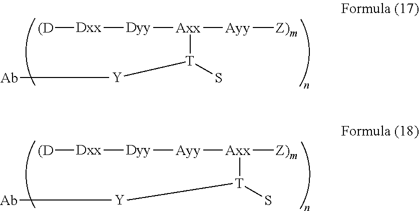
wherein Axx is an amino acid selected from Glu, Apa, Aaa, Dap, Dab, Lys, Orn, Ser, Ama and homo-Lys, with the proviso that Axx in Formula (17) is not in the (D) configuration,
wherein Ayy in Formula (17) is an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24,
wherein Ayy in Formula (18) is an amino acid selected from Phe, homo-Phe, Ala, Trp, Phg, Leu, Val, Tyr and Ser, with the proviso that Ayy in Formula (18) is not in the (D) configuration,
wherein Dxx is a single covalent bond or an amino acid having a hydrophobic side chain,
wherein Dyy represents a single covalent bond, Phe or an amino acid having a basic side chain, with the proviso that if Dxx is an amino acid having a hydrophobic side chain, Dyy is Phe or an amino acid having a basic side chain, and if Dxx is a single covalent bond, Dyy is a single covalent bond, Phe or an amino acid having a basic side chain,
wherein Y is a divalent group comprising one or more atoms selected from C, N, O, P and S,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of Ayy or Axx selected from —OH and —N(H)(R), wherein R represents a hydrogen atom, an alkyl group or a cycloalkyl group, and
wherein Ab, D, m and n are as defined in claim 1.

26. The antibody-drug-conjugate of claim 25, wherein at least one, e.g., one, two, three, four, five, six, seven or eight, of Axx, Ayy, Dxx, Dyy, D, Z, m and T is/are defined as follows:
(a) Axx is an amino acid selected from Dap, Dab, Lys, Orn and homo-Lys,
(b) Ayy in Formula (17) is an amino acid selected from Phe, homo-Phe, Tyr, homo-Tyr, Tyr(OR₁) and homo-Tyr(OR₁),
(c) Ayy in Formula (18) is an amino acid selected from Phe, home-Phe or Ser,
(d) Dxx is a moiety derived from an amino acid selected from Phe, Val, Tyr, homo-Phe and Ala,
(e) Dyy is a covalent bond or a moiety derived from an amino acid selected from Arg, Lys, Cit, Orn, Dap and Dab,
(f) D is a compound selected from a drug selected from auristatin F (AF), MMAF, exatecan, maytansine, DM1 and DM4,
(g) Z is —OH or —NH₂,
(h) T is represented by Formula (9′): 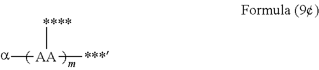
wherein each AA is independently a moiety comprising a trifunctional amino acid,
wherein α indicates covalent attachment to Y,
wherein m is as defined in claim 1,
if m is 1, the side chain of the trifunctional amino acid is covalently attached to S or Axx, the C-terminus being covalently attached to the other moiety S or Axx, respectively,
if m is 2, 3, 4 or 5, **** indicates covalent attachment to Axx, and ***′ indicates covalent attachment to S via the C-terminus of the chain of AA groups,
(i) m is 2 and T is represented by Formula (10′): 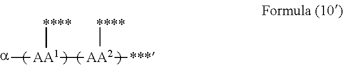
wherein each AA¹ and AA² is independently a moiety comprising a trifunctional amino acid,
wherein α indicates covalent attachment to Y,
wherein **** indicates covalent attachment to Axx, and ***′ indicates covalent attachment to S,
(i) m is 1 and T is represented by Formula (11′): 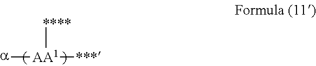
wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(k) m is 1 and T is represented by Formula (12′), or Formula (13′): 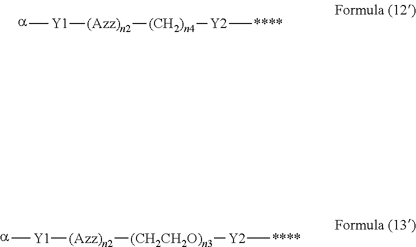
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.

27. The antibody-drug-conjugate of claim 25, wherein at least one, e.g., one, two, three, four, five, six, seven, eight or nine, of Axx, Ayy, Dxx, Dyy, D, Z, m and T is/are defined as follows:
(a) Axx is Lys,
(b) Ayy in Formula (17) is Tyr,
(c) Ayy in Formula (18) is Phe or Ser,
(d) Dxx is Phe or Val,
(e) Dyy is Arg or Cit,
(f) D is a compound selected from a drug selected from AF, MMAF, exatecan, maytansine, DM1 and DM4,
(g) Z is —OH or —NH₂,
(h) m is 1 and T is represented by Formula (11′): 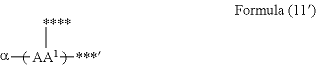
wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(i) m is 1 and T is represented by Formula (12′), or Formula (13′): 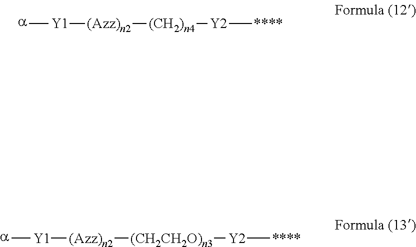
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.

28. The antibody-drug-conjugate of claim 25, wherein, in Formula (17), each Dxx-Dyy-Axx-Ayy is independently selected from Arg-Lys-Phe wherein Dxx is a covalent bond, Arg-Lys-homoPhe wherein Dxx is a covalent bond, Arg-Lys-Tyr wherein Dxx is a covalent bond, Cit-Lys-Phe wherein Dxx is a covalent bond, Cit-Lys-Tyr wherein Dxx is a covalent bond, Arg-Lys-homoTyr wherein Dxx is a covalent bond, Cit-Lys-homo-Tyr wherein Dxx is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr, and wherein, in Formula (18), each Dxx-Dyy-Ayy-Axx is independently selected from Arg-Phe-Lys wherein Dxx is a covalent bond, Arg-Ser-Lys wherein Dxx is a covalent bond, Cit-Phe-Lys wherein Dxx is a covalent bond, Cit-Ser-Lys wherein Dxx is a covalent bond, Cit-homo-Phe-Lys wherein Dxx is a covalent bond, Phe-Cit-Phe-Lys, homoPhe-Cit-Phe-Lys, and Phe-Arg-Phe-Lys.

29. The antibody-drug-conjugate of claim 25, wherein at least one, e.g., one, two, three or four, of D, Z, m and T is/are defined as follows:
(a) D is a compound selected from a drug selected from AF, MMAF, exatecan, maytansine, DM1 and DM4,
(b) Z is —OH or —NH₂,
(c) m is 1 and T is represented by Formula (11′): 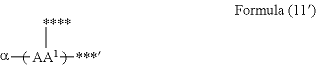
wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(d) m is 1 and T is represented by Formula (12′), or Formula (13′): 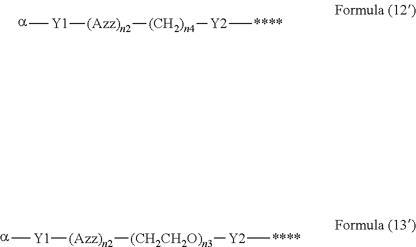
wherein Azz is a single covalent bond or a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.

30. The antibody-drug-conjugate of claim 1, which is represented by one of the following formulae: 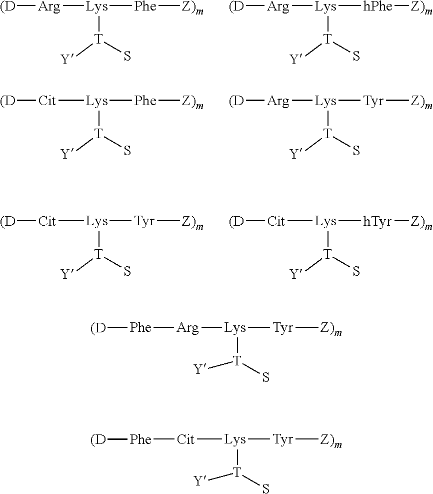
with the proviso that in the above formulae, Lys is not in the (D) configuration,
wherein Y is a divalent group comprising one or more atoms selected from C, N, O, P and S,
wherein T is a (2+m)-valent connecting group; if S is absent, T is a (1+m)-valent connecting group,
wherein S is an atom or group that is optionally present to saturate a free valency of T,
wherein Z represents a group covalently bonded to the C-terminus of an amino acid selected from —OH and —N(H)(R), wherein R represents a hydrogen atom, an alkyl group or a cycloalkyl group, and
wherein Ab, D, m and n are as defined in claim 1.

31. The antibody-drug-conjugate of claim 30, wherein at least one, e.g., one, two, three or four, of D, Z, m and T is/are defined as follows:
(a) D is a compound selected from a drug selected from AF, MMAF, exatecan, maytansine, DM1 and DM4,
(b) Z is —OH or —NH₂,
(c) m is 2 and T is represented by Formula (10′): 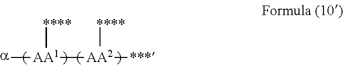
wherein each AA¹ and AA² is independently a moiety comprising a trifunctional amino acid,
wherein α indicates covalent attachment to Y,
wherein **** indicates covalent attachment to Lys, and ***′ indicates covalent attachment to S,
(d) m is 1 and T is represented by Formula (11′): 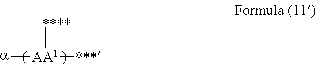
wherein AA′ is a moiety comprising a trifunctional amino acid,
wherein α indicates covalent attachment to Y,
wherein the side chain of the trifunctional amino acid is covalently attached to Lys or S, the C-terminus is covalently attached to the other moiety S or Lys, respectively,
(e) m is 1 and T is represented by Formula (12′), or Formula (13′): 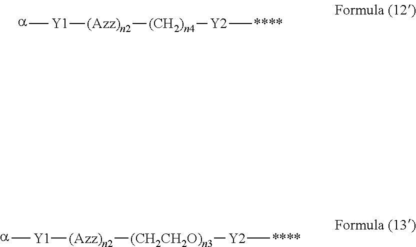
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Lys.

32. The antibody-drug-conjugate of claim 30, wherein D, Z, m and T are defined as follows:
(a) D is DM1,
(b) Z is —OH or —NH₂,
(c) m is 1 and T is represented by Formula (13′): 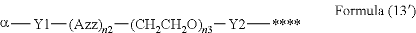
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Lys.

33. The antibody-drug-conjugate of claim 30, wherein D, Z, m and T is/are defined as follows:
(a) D is AF,
(b) Z is —OH or —NH₂,
(c) m is 1 and T is represented by Formula (13′): 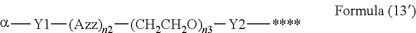
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Lys.

34. The antibody-drug-conjugate of claim 1, which is represented by Formula (19), Formula (20), Formula (21), or Formula (22): 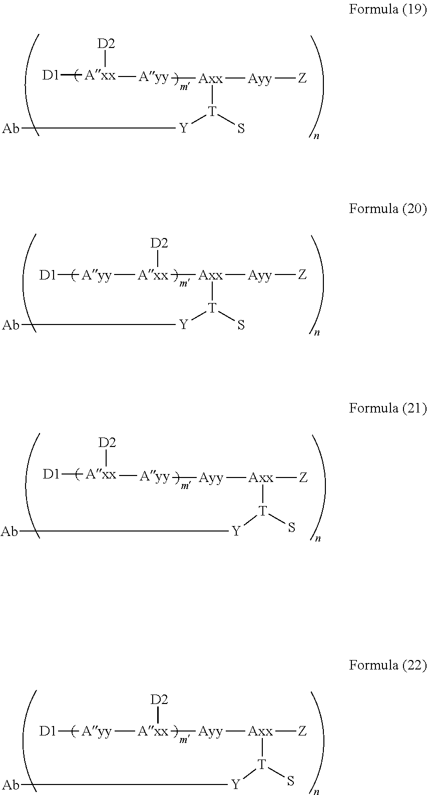
wherein Axx is a trifunctional amino acid, with the proviso that Axx in Formula (19) and Formula (20) is not an amino acid in the (D) configuration,
wherein Ayy is an amino acid selected from Phe, Ala, Trp, Tyr, Phg, Met, Val, His, Lys, Arg, Cit, Abu, Orn, Ser, Thr, Leu and Ile, or Ayy in Formula (19) and Formula (20) is an amino acid selected from homo-Tyr, homo-Phe, beta-Phe and beta-homo-Phe, Tyr(OR₁) and homoTyr(OR₁) wherein R₁ is —(CH₂CH₂O)ₙ₁—R₂, wherein R₂ is a hydrogen atom or a methyl group and n1 is an integer of 2 to 24, with the proviso that Ayy in Formula (21) and Formula (22) is not an amino acid in the (D) configuration,
wherein each A″yy is independently an amino acid selected from Phe, Ala, Trp, Tyr, Phg, Met, Val, His, Lys, Arg, Cit, Abu, Orn, with the proviso that A″yy in Formula (20) and Formula (22) is not an amino acid in the (D) configuration,
wherein Y is a divalent group comprising one or more atoms selected from C, N, O, P and S,
wherein T is a tri-valent connecting group; if S is absent, T is a divalent connecting group,
wherein S is an atom or group that is optionally present to saturate a free valency of T,
wherein Z represents a group covalently bonded to the C-terminus of Ayy in Formula (19) and Formula (20) or to the C-terminus of Axx in Formula (21) or Formula (22), which is selected from —OH and —N(H)(R), wherein R represents a hydrogen atom, an alkyl group or a cycloalkyl group,
wherein D1 is an antitumor compound,
wherein m′ is (m−1), m being as defined in claim 1, with the proviso that m′ is not 0,
if m′ is 1, A″xx is a trifunctional amino acid with the proviso that A″xx in Formula (19) and Formula (21) is not an amino acid in the (D) configuration, and D2 is an antitumor compound,
if m′ is more than 1, each D2 is independently selected from a hydrogen atom and an antitumor compound, wherein multiple moieties D2 can be the same or different with the proviso that at least one D2 is not a hydrogen atom; if D2 is a hydrogen atom, A″xx is an amino acid with the proviso that A″xx in Formula (19) and Formula (21) is not an amino acid in the (D) configuration; if D2 is an antitumor compound, A″xx is a trifunctional amino acid with the proviso that A″xx in Formula (19) and Formula (21) is not an amino acid in the (D) configuration,
wherein Ab and n are as defined in claim 1.

35. The antibody-drug-conjugate of claim 34, wherein at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Orn and homo-Lys,
(b) Ayy in Formula (19) and Formula (20) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is independently a compound selected from a drug selected from AF, MMAF, exatecan, maytansine, DM1 and DM4,
(f) Z is —OH or —NH₂,
(g) m is 1 and T is represented by Formula (11′): 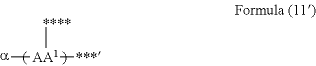
wherein AA′ is a moiety comprising a trifunctional amino acid,
wherein α indicates covalent attachment to Y,
wherein the side chain of the trifunctional amino acid is covalently attached to Axx or S, the C-terminus is covalently attached to the other moiety S or Axx, respectively,
(h) m is 1 and T is represented by Formula (12′), or Formula (13′): 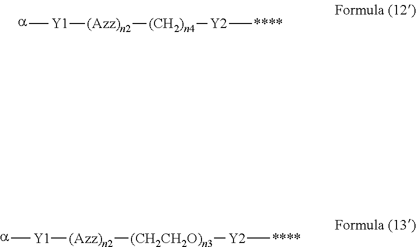
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein n4 is an integer of 1 to 50,
wherein α indicates covalent attachment to Y, and
wherein **** indicates covalent attachment to Axx.

36. The antibody-drug-conjugate of claim 34, wherein at least one, e.g., one, two, three, four, five, six, seven, eight or nine, of Axx, Ayy, A″xx, A″yy, D1, D2, Z, m′ and T is/are defined as follows:
(a) Axx is an amino acid selected from Dap, Dab, Lys, Orn and homo-Lys,
(b) Ayy in Formula (17) is an amino acid selected from Phe, homo-Phe, Tyr, homo-Tyr, Tyr(OR₁) and homo-Tyr(OR₁),
(c) A″xx is an amino acid selected from Lys, homo-Lys, Cit, Orn, Dap and Dab,
(d) A″yy is an amino acid selected from Phe, Ala, Trp, Phg and Tyr,
(e) each D1 and D2 is AF,
(f) Z is —OH or —NH₂,
(g) m′ is 1 and T is represented by Formula (13′): 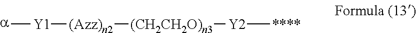
wherein Azz is a moiety comprising one or more solubilizing groups,
wherein Y1 is a single covalent bond, an alkyl group having 1 to 6 carbon atoms, a carbonyl-containing group, or an amino-containing group,
wherein Y2 is a single covalent bond, a carbonyl-containing group, or an amino-containing group,
wherein n2 is an integer of 0 to 5,
wherein n3 is an integer of 1 to 50,
wherein α indicates covalent attachment to Y, and
wherein **** indicates covalent attachment to Axx.

37. The antibody-drug-conjugate of claim 1 or 23, wherein the antitumor compound (D) is selected from DNA-alkylating agents, topoisomerase inhibitors, RNA-polymerase II inhibitors, DNA-cleaving agents, antimitotic agents or microtubule disruptors, anti-metabolites, Kinesin spindle protein inhibitors, kinase inhibitors, nicotinamide phosphoribosyl transferase inhibitors, matrix metallopeptidase 9 inhibitors, phosphatase inhibitors, or radioisotopes and/or pharmaceutically acceptable salts thereof; if more than one D is present, each D is independently selected from the aforementioned compounds.

38. The antibody-drug-conjugate of claim 1 or 23, wherein the antitumor compound D is selected from amanitin, duocarmycin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof; if more than one D is present, each D is independently selected from the aforementioned compounds.

39. The antibody-drug-conjugate of claim 1, which is selected from the following compounds: 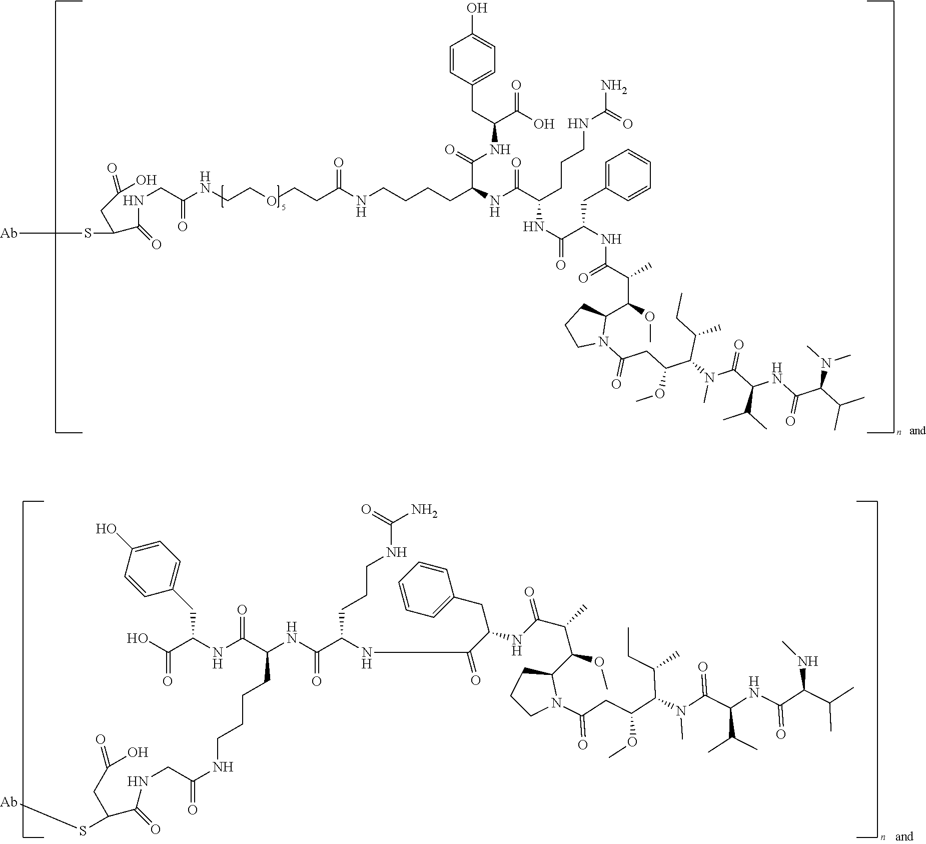 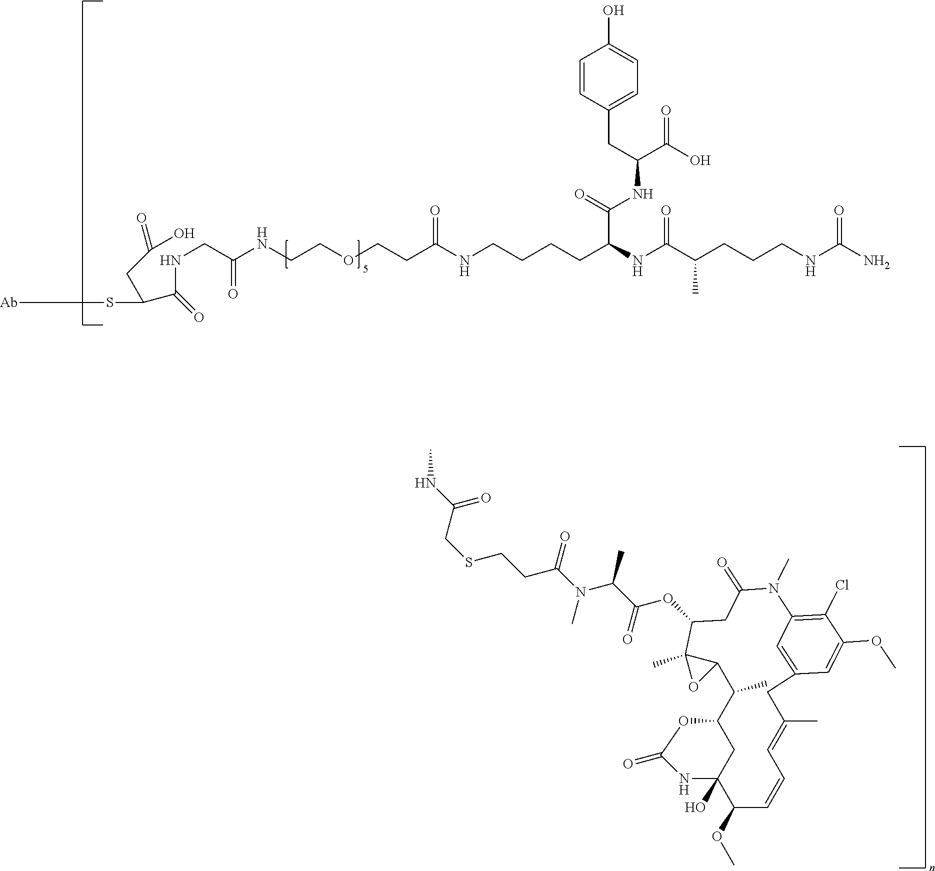
wherein Ab and n are as defined in claim 1.

40. A pharmaceutical composition comprising the antibody-drug conjugate of claim 1 or 23 or a pharmaceutically acceptable salt thereof, and a pharmaceutically acceptable carrier.

41. The pharmaceutical composition of claim 40, wherein the pharmaceutical composition is for use in treating or imaging cancer.

42. The pharmaceutical composition for use of claim 41, wherein the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.

43. A method for treating cancer, the method comprising administering an effective amount of the antibody-drug conjugate of claim 1 or 23 or a pharmaceutically acceptable salt thereof to a subject in need thereof.

44. The method of claim 43, wherein the cancer is one or more selected from the group consisting of breast cancer, liver cancer, skin cancer, ovarian cancer, cervical cancer, prostate cancer, testicular cancer, brain cancer, clear cell renal cell carcinoma, glioma, melanoma, lung cancer, non-small cell lung cancer (NSCLC), small cell lung cancer, pancreatic cancer, gastric cancer, acute myeloid leukemia (AML), Hodgkin's lymphoma, non-Hodgkin's lymphoma (NHL), colorectal cancer, colon cancer, renal cancer, esophageal cancer, leukaemia, hepatocellular carcinoma, bone cancer, bladder cancer, sarcomas, kidney cancer, head and neck cancer, hypopharyngeal squamous cell carcinoma, glioblastoma, neuroblastoma, endometrial cancer, and urothelial cell carcinoma.


SEQUENCE LISTING (12 sequences)

SEQ ID NO: 1 - protein, 10 aa, synthetic construct
GFTFSDYYMS

SEQ ID NO: 2 - protein, 17 aa, synthetic construct
YTSSSGSTIYYADSVKG

SEQ ID NO: 3 - protein, 7 aa, synthetic construct
DITAFDI

SEQ ID NO: 4 - protein, 14 aa, synthetic construct
TGTSSDVGGYNYVS

SEQ ID NO: 5 - protein, 7 aa, synthetic construct
EVNKRPS

SEQ ID NO: 6 - protein, 10 aa, synthetic construct
CSYAGSNNYV

SEQ ID NO: 7 - protein, 116 aa, synthetic construct
EVQLVQSGGGLVKPGGSLRLSCAASGFTFSDYYMSWIRQAPGKGLEWVSYTSSSGSTIYYADSVKGRFTISRDNAKNSLYLQMNSLRAEDTAVYYCARDITAFDIWGQGTMVTVSS

SEQ ID NO: 8 - protein, 110 aa, synthetic construct
LPVLTQPPSASGSPGQSVTISCTGTSSDVGGYNYVSWYQQHPGKAPKLMIYEVNKRPSGVPDRFSGSKSGNTASLTVSGLQAEDEADYYCCSYAGSNNYVFGTGTKVTVL

SEQ ID NO: 9 - protein, 443 aa, synthetic construct
EVQLVQSGGGLVKPGGSLRLSCAASGFTFSDYYMSWIRQAPGKGLEWVSYTSSSGSTIYYADSVKGRFTISRDNAKNSLYLQMNSLRAEDTAVYYCARDITAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 446 aa, synthetic construct
EVQLVQSGGGLVKPGGSLRLSCAASGFTFSDYYMSWIRQAPGKGLEWVSYTSSSGSTIYYADSVKGRFTISRDNAKNSLYLQMNSLRAEDTAVYYCARDITAFDIWGQGTMVTVSSASTKGPSVFPLAPSSKSTSGGTAALGCLVNDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 216 aa, synthetic construct
LPVLTQPPSASGSPGQSVTISCTGTSSDVGGYNYVSWYQQHPGKAPKLMIYEVNKRPSGVPDRFSGSKSGNTASLTVSGLQAEDEADYYCCSYAGSNNYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 446 aa, synthetic construct
EVQLVQSGGGLVKPGGSLRLSCAASGFTFSDYYMSWIRQAPGKGLEWVSYTSSSGSTIYYADSVKGRFTISRDNAKNSLYLQMNSLRAEDTAVYYCARDITAFDIWGQGTMVTVSSASTKGPSVFPLAPSSKSTSGGTAALGCLVNDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK